(19) United States
(12) Patent Application Publication
(10) Pub. No.: US 2016/0237430 A1
(43) Pub. Date: Aug. 18, 2016

(54) ALLELE-SPECIFIC RNA SILENCING FOR THE TREATMENT OF HYPERTROPHIC CARDIOMYOPATHY

(71) Applicants: PRESIDENT AND FELLOWS OF HARVARD COLLEGE, Cambridge, MA (US); THE BRIGHAM AND WOMEN'S HOSPITAL, INC., Boston, MA (US)

(72) Inventors: Jonathan Seidman, Milton, MA (US); Christine Seidman, Milton, MA (US); Jiangmingjian Jiang, Boston, MA (US); Hiroko Wakimoto, Newton, MA (US)

(21) Appl. No.: 15/024,288

(22) PCT Filed: Sep. 23, 2014
(86) PCT No.: PCT/US2014/056987

§ 371 Date: Mar. 23, 2016

Related U.S. Application Data
(60) Provisional application No. 61/881,264, filed on Sep. 23, 2013.

(51) Int. Cl. C12N 15/113

(52) U.S. Cl. CPC C12N 15/113; C12N 2310/14; C12N 2320/34

(57) ABSTRACT

Provided herein are methods and compositions useful for the treatment of hypertrophic cardiomyopathy (HCM), dilated cardiomyopathy (DCM) and/or left ventricular non-compaction (LVNC) and other cardiomyopathies through allele-specific RNA silencing.

### RELATED APPLICATIONS

This application claims the benefit of priority to U.S. Provisional Patent Application Ser. No. 61/881,264, filed Sep. 23, 2013, which is hereby incorporated by reference in their entirety.

### GOVERNMENT INTEREST

This invention was made with Government support under National Institutes of Health Grants U01 HL098166; R01 HL084553. The Government has certain rights in the invention.

### BACKGROUND

Hypertrophic cardiomyopathy (HCM) is an autosomal dominant disease characterized by an increase in left ventricular wall thickness (LVWT), disorganization of cardiomyocytes and expansion of myocardial fibrosis that occurs in the absence of systemic disease. HCM is the leading cause of non-violent sudden death in young adults and the most common cause of sudden death on the athletic field. HCM is caused by mutations in genes that encode protein constituents of the cardiac sarcomere, the contractile unit of muscle. More than 1000 distinct pathogenic mutations have been identified, and over half of these occur in MYH7 (encoding 0 myosin heavy chain) and MYBPC (encoding myosin binding protein-C). Most HCM mutations are missense mutations, producing amino acid substitutions in myosin that perturb the sarcomere's contractile function.

One exemplary HCM-causing missense mutation found in MYH7 results in a substitution of glutamine for arginine at position 403 of the encoded protein (MYH7 R403Q). MYH7 R403Q causes particularly severe disease that is characterized by early-onset and progressive myocardial dysfunction and a high incidence of sudden cardiac death. Myh6 in mice and MYH7 in humans are highly homologous in sequence and encode the predominant myosin isoforms in the adult hearts. Mice heterozygous for a mutation in Myh6 analogous to MYH7 R403Q and In some embodiments, under the control of the endogenous Myh locus (MHC^{403/+} mice) recapitulate human HCM and develop hypertrophy, myocyte disarray and increased myocardial fibrosis. Analyses of mutant myosins isolated from MHC^{403/+} mice showed that HCM mutations cause fundamental changes in sarcomere functions, including increased acto-myosin sliding velocity, force generation, and ATP hydrolysis. These changes in turn alter calcium cycling and gene transcription in myocytes and ultimately induce pathologic remodeling of the heart in vivo. Understanding this pathogenic cascade has led to the identification of secondary signaling molecules as potential therapeutic targets. However, no strategies have been defined that correct the primary biophysical and biochemical abnormalities of sarcomeres with HCM mutations.

### SUMMARY

Provided herein are methods and compositions useful for the treatment of hypertrophic cardiomyopathy (HCM), dilated cardiomyopathy (DCM) and/or left ventricular non-compaction (LVNC) and other cardiomyopathies caused by dominant acting poison polypeptides through allele-specific RNA silencing.

In some embodiments, provided herein is a method of preventing or treating HCM, DCM or LVNC in a subject who has in their genome a first MYH7 allele containing an HCM-causing mutation, a DCM-causing mutation or a LVNC-causing mutation (e.g., an HCM-causing mutation, a DCM-causing mutation or a LVNC-causing mutation listed in FIG. 8, such as an R403Q mutation). In certain embodiments, the subject further comprises a second MYH7 allele that does not contain an HCM-causing mutation, a DCM-causing mutation or a LVNC-causing mutation. In some embodiments, the method comprises administering to the subject an interfering RNA molecule (e.g., a siRNA molecule or a shRNA molecule) that selectively inactivates the transcript encoded by the first MYH7 allele compared to the transcript encoded by the second MYH7 allele (e.g., the interfering RNA molecule inactivates the transcript of the first MYH7 allele at least 1.5, 2, 2.5, or 3 times as much as it inactivates the second MYH7 allele). In some embodiments, the method further comprises the step of sequencing the first MYH7 allele and the second MYH7 allele before administering to the subject the interfering RNA molecule. In some embodiments, the inhibitory RNA molecule is an inhibitory RNA molecule disclosed or contemplated herein.

In some embodiments, the method comprises administering to the subject more than one different interfering RNA molecule, wherein each interfering RNA molecule selectively inactivates the transcript encoded by the first MYH7 allele compared to the transcript encoded by the second MYH7 allele. In some embodiments, each interfering RNA molecule targets a different polymorphism or mutation present on the transcript encoded by the first MYH7 allele but that is not present on the transcript encoded by the second MYH7 allele.

In some embodiments, the interfering RNA molecule targets a polymorphism or mutation present on the transcript encoded by the first MYH7 allele but that is not present on the transcript encoded by the second MYH7 allele (e.g., a polymorphism or mutation listed in FIG. 7). In some embodiments, the interfering RNA molecule targets the HCM-causing mutation, DCM-causing mutation or LVNC-causing mutation. In some embodiments, the interfering RNA molecule targets a polymorphism present on the transcript encoded by the first MYH7 allele that is not the HCM-causing mutation, DCM-causing mutation or LVNC-causing mutation.

In certain embodiments, the interfering RNA molecule comprises a nucleic acid sequence of 21 nucleotides in length, wherein 20 nucleotides of the nucleic acid sequence are complementary to the transcript of the first MYH7 allele and no more than 19 nucleotides of the nucleic acid sequence are complementary to the transcript of the second MYH7 allele. In some embodiments, the interfering RNA molecule comprises a nucleotide sequence that is complementary to a nucleotide sequence of the first allele that includes the polymorphism or mutation except for a single nucleotide mismatch at a position outside of the polymorphism or mutation. In some embodiments, the inhibitory RNA molecule is delivered in a vector that has a tropism for cardiac tissue. In some embodiments, the vector is an adeno-associated virus (AAV). In some embodiments, expression of the inhibitory RNA molecule and/or the vector is driven by a cardiac-specific promoter. In certain embodiments, the cardiac-specific promoter is a cardiac specific troponin T promoter.

In some embodiments, provided herein is a method of preventing or treating HCM in a subject who has in their genome a first MYL2 allele containing an HCM-causing mutation (e.g., an HCM-causing mutation listed in FIG. 10). In certain embodiments, the subject has in their genome a second MYL2 allele that does not contain an HCM-causing mutation. In some embodiments, the method comprises administering to the subject an interfering RNA molecule (e.g., a siRNA molecule or a shRNA molecule) that selectively inactivates the transcript encoded by the first MYL2 allele compared to the transcript encoded by the second MYL2 allele (e.g., the interfering RNA molecule inactivates the transcript of the first MYL2 allele at least 1.5, 2, 2.5, or 3 times as much as it inactivates the second MYL2 allele). In some embodiments, the method further comprises the step of sequencing the first MYL2 allele and the second MYL2 allele before administering to the subject the interfering RNA molecule. In some embodiments, the inhibitory RNA molecule is an inhibitory RNA molecule disclosed or contemplated herein.

In some embodiments, the method comprises administering to the subject more than one different interfering RNA molecule, wherein each interfering RNA molecule selectively inactivates the transcript encoded by the first MYL2 allele compared to the transcript encoded by the second MYL2 allele. In some embodiments, each interfering RNA molecule targets a different polymorphism or mutation present on the transcript encoded by the first MYL2 allele but that is not present on the transcript encoded by the second MYL2 allele.

In some embodiments, the interfering RNA molecule targets a polymorphism or mutation present on the transcript encoded by the first MYL2 allele but that is not present on the transcript encoded by the second MYL2 allele (e.g., a polymorphism or mutation listed in FIG. 9). In some embodiments, the interfering RNA molecule targets the HCM-causing mutation, DCM-causing mutation or LVNC-causing mutation. In some embodiments, the interfering RNA molecule targets a polymorphism present on the transcript encoded by the first MYL2 allele that is not the HCM-causing mutation.

In certain embodiments, the interfering RNA molecule comprises a nucleic acid sequence of 21 nucleotides in length, wherein 20 nucleotides of the nucleic acid sequence are complementary to the transcript of the first MYL2 allele and no more than 19 nucleotides of the nucleic acid sequence are complementary to the transcript of the second MYL2 allele. In some embodiments, the interfering RNA molecule comprises a nucleotide sequence that is complementary to a nucleotide sequence of the first allele that includes the polymorphism or mutation except for a single nucleotide mismatch at a position outside of the polymorphism or mutation. In some embodiments, the inhibitory RNA molecule is delivered in a vector that has a tropism for cardiac tissue. In some embodiments, the vector is an adeno-associated virus (AAV). In some embodiments, expression of the inhibitory RNA molecule and/or the vector is driven by a cardiac-specific promoter. In certain embodiments, the cardiac-specific promoter is a cardiac specific troponin T promoter.

In some embodiments, provided herein is a method of preventing or treating HCM in a subject who has in their genome a first MYL3 allele containing an HCM-causing mutation (e.g., an HCM-causing mutation listed in FIG. 12). In certain embodiments, the subject has in their genome a second MYL3 allele that does not contain an HCM-causing mutation. In some embodiments, the method comprises administering to the subject an interfering RNA molecule (e.g., a siRNA molecule or a shRNA molecule) that selectively inactivates the transcript encoded by the first MYL3 allele compared to the transcript encoded by the second MYL3 allele (e.g., the interfering RNA molecule inactivates the transcript of the first MYL3 allele at least 1.5, 2, 2.5, or 3 times as much as it inactivates the second MYL3 allele). In some embodiments, the method further comprises the step of sequencing the first MYL3 allele and the second MYL3 allele before administering to the subject the interfering RNA molecule. In some embodiments, the inhibitory RNA molecule is an inhibitory RNA molecule disclosed or contemplated herein.

In some embodiments, the method comprises administering to the subject more than one different interfering RNA molecule, wherein each interfering RNA molecule selectively inactivates the transcript encoded by the first MYL3 allele compared to the transcript encoded by the second MYL3 allele. In some embodiments, each interfering RNA molecule targets a different polymorphism or mutation present on the transcript encoded by the first MYL3 allele but that is not present on the transcript encoded by the second MYL3 allele.

In some embodiments, the interfering RNA molecule targets a polymorphism or mutation present on the transcript encoded by the first MYL3 allele but that is not present on the transcript encoded by the second MYL3 allele (e.g., a polymorphism or mutation listed in FIG. 11). In some embodiments, the interfering RNA molecule targets the HCM-causing mutation, DCM-causing mutation or LVNC-causing mutation. In some embodiments, the interfering RNA molecule targets a polymorphism present on the transcript encoded by the first MYL3 allele that is not the HCM-causing mutation.

In certain embodiments, the interfering RNA molecule comprises a nucleic acid sequence of 21 nucleotides in length, wherein 20 nucleotides of the nucleic acid sequence are complementary to the transcript of the first MYL3 allele and no more than 19 nucleotides of the nucleic acid sequence are complementary to the transcript of the second MYL3 allele. In some embodiments, the interfering RNA molecule comprises a nucleotide sequence that is complementary to a nucleotide sequence of the first allele that includes the polymorphism or mutation except for a single nucleotide mismatch at a position outside of the polymorphism or mutation. In some embodiments, the inhibitory RNA molecule is delivered in a vector that has a tropism for cardiac tissue. In some embodiments, the vector is an adeno-associated virus (AAV). In some embodiments, expression of the inhibitory RNA molecule and/or the vector is driven by a cardiac-specific promoter. In certain embodiments, the cardiac-specific promoter is a cardiac specific troponin T promoter.

In some embodiments, provided herein is a method of preventing or treating HCM or DCM in a subject who has in their genome a first TNNI3 allele containing an HCM-causing mutation or a DCM-causing mutation (e.g., an HCM-causing mutation or a DCM-causing mutation listed in FIG. 14). In certain embodiments, the subject has in their genome a second TNNI3 allele that does not contain an HCM-causing mutation or a DCM-causing mutation. In some embodiments, the method comprises administering to the subject an interfering RNA molecule (e.g., a siRNA molecule or a shRNA molecule) that selectively inactivates the transcript encoded by the first TNNI3 allele compared to the transcript encoded by the second TNNI3 allele (e.g., the interfering RNA molecule inactivates the transcript of the first TNNI3 allele at least 1.5, 2, 2.5, or 3 times as much as it inactivates the second TNNI3 allele). In some embodiments, the method further comprises the step of sequencing the first TNNI3 allele and the second TNNI3 allele before administering to the subject the interfering RNA molecule. In some embodiments, the inhibitory RNA molecule is an inhibitory RNA molecule disclosed or contemplated herein.

In some embodiments, the method comprises administering to the subject more than one different interfering RNA molecule, wherein each interfering RNA molecule selectively inactivates the transcript encoded by the first TNNI3 allele compared to the transcript encoded by the second TNNI3 allele. In some embodiments, each interfering RNA molecule targets a different polymorphism or mutation present on the transcript encoded by the first TNNI3 allele but that is not present on the transcript encoded by the second TNNI3 allele.

In some embodiments, the interfering RNA molecule targets a polymorphism or mutation present on the transcript encoded by the first TNNI3 allele but that is not present on the transcript encoded by the second TNNI3 allele (e.g., a polymorphism or mutation listed in FIG. 13). In some embodiments, the interfering RNA molecule targets the HCM-causing mutation or DCM-causing mutation. In some embodiments, the interfering RNA molecule targets a polymorphism present on the transcript encoded by the first TNNI3 allele that is not the HCM-causing mutation or DCM-causing mutation.

In certain embodiments, the interfering RNA molecule comprises a nucleic acid sequence of 21 nucleotides in length, wherein 20 nucleotides of the nucleic acid sequence are complementary to the transcript of the first TNNI3 allele and no more than 19 nucleotides of the nucleic acid sequence are complementary to the transcript of the second TNNI3 allele. In some embodiments, the interfering RNA molecule comprises a nucleotide sequence that is complementary to a nucleotide sequence of the first allele that includes the polymorphism or mutation except for a single nucleotide mismatch at a position outside of the polymorphism or mutation. In some embodiments, the inhibitory RNA molecule is delivered in a vector that has a tropism for cardiac tissue. In some embodiments, the vector is an adeno-associated virus (AAV). In some embodiments, expression of the inhibitory RNA molecule and/or the vector is driven by a cardiac-specific promoter. In certain embodiments, the cardiac-specific promoter is a cardiac specific troponin T promoter. In some embodiments, provided herein is a method of preventing or treating HCM or DCM in a subject who has in their genome a first TNNT2 allele containing an HCM-causing mutation or a DCM-causing mutation (e.g., an HCM-causing mutation or a DCM-causing mutation listed in FIG. 16). In certain embodiments, the subject has in their genome a second TNNT2 allele that does not contain an HCM-causing mutation or a DCM-causing mutation. In some embodiments, the method comprises administering to the subject an interfering RNA molecule (e.g., a siRNA molecule or a shRNA molecule) that selectively inactivates the transcript encoded by the first TNNT2 allele compared to the transcript encoded by the second TNNT2 allele (e.g., the interfering RNA molecule inactivates the transcript of the first TNNT2 allele at least 1.5, 2, 2.5, or 3 times as much as it inactivates the second TNNT2 allele). In some embodiments, the method further comprises the step of sequencing the first TNNT2 allele and the second TNNT2 allele before administering to the subject the interfering RNA molecule. In some embodiments, the inhibitory RNA molecule is an inhibitory RNA molecule disclosed or contemplated herein.

In some embodiments, the method comprises administering to the subject more than one different interfering RNA molecule, wherein each interfering RNA molecule selectively inactivates the transcript encoded by the first TNNT2 allele compared to the transcript encoded by the second TNNT2 allele. In some embodiments, each interfering RNA molecule targets a different polymorphism or mutation present on the transcript encoded by the first TNNT2 allele but that is not present on the transcript encoded by the second TNNT2 allele.

In some embodiments, the interfering RNA molecule targets a polymorphism or mutation present on the transcript encoded by the first TNNT2 allele but that is not present on the transcript encoded by the second TNNT2 allele (e.g., a polymorphism or mutation listed in FIG. 15). In some embodiments, the interfering RNA molecule targets the HCM-causing mutation or DCM-causing mutation. In some embodiments, the interfering RNA molecule targets a polymorphism present on the transcript encoded by the first TNNT2 allele that is not the HCM-causing mutation or DCM-causing mutation.

In certain embodiments, the interfering RNA molecule comprises a nucleic acid sequence of 21 nucleotides in length, wherein 20 nucleotides of the nucleic acid sequence are complementary to the transcript of the first TNNT2 allele and no more than 19 nucleotides of the nucleic acid sequence are complementary to the transcript of the second TNNT2 allele. In some embodiments, the interfering RNA molecule comprises a nucleotide sequence that is complementary to a nucleotide sequence of the first allele that includes the polymorphism or mutation except for a single nucleotide mismatch at a position outside of the polymorphism or mutation. In some embodiments, the inhibitory RNA molecule is delivered in a vector that has a tropism for cardiac tissue. In some embodiments, the vector is an adeno-associated virus (AAV). In some embodiments, expression of the inhibitory RNA molecule and/or the vector is driven by a cardiac-specific promoter. In certain embodiments, the cardiac-specific promoter is a cardiac specific troponin T promoter.

In some embodiments, provided herein is a method of preventing or treating HCM or DCM in a subject who has in their genome a first TMP1 allele containing an HCM-causing mutation or a DCM-causing mutation (e.g., an HCM-causing mutation or a DCM-causing mutation listed in FIG. 18). In certain embodiments, the subject has in their genome a second TMP1 allele that does not contain an HCM-causing mutation or a DCM-causing mutation. In some embodiments, the method comprises administering to the subject an interfering RNA molecule (e.g., a siRNA molecule or a shRNA molecule) that selectively inactivates the transcript encoded by the first TMP1 allele compared to the transcript encoded by the second TMP1 allele (e.g., the interfering RNA molecule inactivates the transcript of the first TMP1 allele at least 1.5, 2, 2.5, or 3 times as much as it inactivates the second TMP1 allele). In some embodiments, the method further comprises the step of sequencing the first TMP1 allele and the second TMP1 allele before administering to the subject the interfering RNA molecule. In some embodiments, the inhibitory RNA molecule is an inhibitory RNA molecule disclosed or contemplated herein.

In some embodiments, the method comprises administering to the subject more than one different interfering RNA molecule, wherein each interfering RNA molecule selectively inactivates the transcript encoded by the first TMP1 allele compared to the transcript encoded by the second TMP1 allele. In some embodiments, each interfering RNA molecule targets a different polymorphism or mutation present on the transcript encoded by the first TMP1 allele but that is not present on the transcript encoded by the second TMP1 allele.

In some embodiments, the interfering RNA molecule targets a polymorphism or mutation present on the transcript encoded by the first TMP1 allele but that is not present on the transcript encoded by the second TMP1 allele (e.g., a polymorphism or mutation listed in FIG. 17). In some embodiments, the interfering RNA molecule targets the HCM-causing mutation or DCM-causing mutation. In some embodiments, the interfering RNA molecule targets a polymorphism present on the transcript encoded by the first TMP1 allele that is not the HCM-causing mutation or DCM-causing mutation.

In certain embodiments, the interfering RNA molecule comprises a nucleic acid sequence of 21 nucleotides in length, wherein 20 nucleotides of the nucleic acid sequence are complementary to the transcript of the first TMP1 allele and no more than 19 nucleotides of the nucleic acid sequence are complementary to the transcript of the second TMP1 allele. In some embodiments, the interfering RNA molecule comprises a nucleotide sequence that is complementary to a nucleotide sequence of the first allele that includes the polymorphism or mutation except for a single nucleotide mismatch at a position outside of the polymorphism or mutation. In some embodiments, the inhibitory RNA molecule is delivered in a vector that has a tropism for cardiac tissue. In some embodiments, the vector is an adeno-associated virus (AAV). In some embodiments, expression of the inhibitory RNA molecule and/or the vector is driven by a cardiac-specific promoter. In certain embodiments, the cardiac-specific promoter is a cardiac specific troponin T promoter.

In some embodiments, provided herein is a method of preventing or treating HCM or DCM in a subject who has in their genome a first ACTC1 allele containing an HCM-causing mutation or a DCM-causing mutation (e.g., an HCM-causing mutation or a DCM-causing mutation listed in FIG. 20). In certain embodiments, the subject has in their genome a second ACTC1 allele that does not contain an HCM-causing mutation or a DCM-causing mutation. In some embodiments, the method comprises administering to the subject an interfering RNA molecule (e.g., a siRNA molecule or a shRNA molecule) that selectively inactivates the transcript encoded by the first ACTC1 allele compared to the transcript encoded by the second ACTC1 allele (e.g., the interfering RNA molecule inactivates the transcript of the first ACTC1 allele at least 1.5, 2, 2.5, or 3 times as much as it inactivates the second ACTC1 allele). In some embodiments, the method further comprises the step of sequencing the first ACTC1 allele and the second ACTC1 allele before administering to the subject the interfering RNA molecule. In some embodiments, the inhibitory RNA molecule is an inhibitory RNA molecule disclosed or contemplated herein.

In some embodiments, the method comprises administering to the subject more than one different interfering RNA molecule, wherein each interfering RNA molecule selectively inactivates the transcript encoded by the first ACTC1 allele compared to the transcript encoded by the second ACTC1 allele. In some embodiments, each interfering RNA molecule targets a different polymorphism or mutation present on the transcript encoded by the first ACTC1 allele but that is not present on the transcript encoded by the second ACTC1 allele.

In some embodiments, the interfering RNA molecule targets a polymorphism or mutation present on the transcript encoded by the first ACTC1 allele but that is not present on the transcript encoded by the second ACTC1 allele (e.g., a polymorphism or mutation listed in FIG. 19). In some embodiments, the interfering RNA molecule targets the HCM-causing mutation or DCM-causing mutation. In some embodiments, the interfering RNA molecule targets a polymorphism present on the transcript encoded by the first ACTC1 allele that is not the HCM-causing mutation or DCM-causing mutation.

In certain embodiments, the interfering RNA molecule comprises a nucleic acid sequence of 21 nucleotides in length, wherein 20 nucleotides of the nucleic acid sequence are complementary to the transcript of the first ACTC1 allele and no more than 19 nucleotides of the nucleic acid sequence are complementary to the transcript of the second ACTC1 allele. In some embodiments, the interfering RNA molecule comprises a nucleotide sequence that is complementary to a nucleotide sequence of the first allele that includes the polymorphism or mutation except for a single nucleotide mismatch at a position outside of the polymorphism or mutation. In some embodiments, the inhibitory RNA molecule is delivered in a vector that has a tropism for cardiac tissue. In some embodiments, the vector is an adeno-associated virus (AAV). In some embodiments, expression of the inhibitory RNA molecule and/or the vector is driven by a cardiac-specific promoter. In certain embodiments, the cardiac-specific promoter is a cardiac specific troponin T promoter.

In certain embodiments, provided herein is an interfering RNA molecule (e.g., a siRNA molecule or a shRNA molecule) that selectively inhibits expression of a MYH7 transcript comprising a MYH7 polymorphism or mutation compared to a MYH7 transcript not comprising the MYH7 polymorphism or mutation (e.g., a polymorphism or mutation listed in FIG. 7 and/or FIG. 8). In some embodiments, the interfering RNA molecule inactivates the transcript comprising a MYH7 polymorphism or mutation at least 1.5 times, 2 times, 2.5 times or 3 times as much as it inactivates the transcript not comprising the MYH7 polymorphism or mutation. In some embodiments, the interfering RNA molecule comprises a nucleotide sequence that is complementary to a nucleotide sequence of the MYH7 transcript comprising the MYH7 polymorphism or mutation except for a single nucleotide mismatch at a position outside of the polymorphism or mutation. In some embodiments, the interfering RNA molecule comprises a nucleic acid sequence of 21 nucleotides in length, wherein 20 nucleotides of the nucleic acid sequence are complementary to the MYH7 transcript comprising the MYH7 polymorphism or mutation and no more than 19 nucleotides of the nucleic acid sequence are complementary to the transcript not comprising the MYH7 polymorphism or mutation.

In certain embodiments, provided herein is an interfering RNA molecule (e.g., a siRNA molecule or a shRNA molecule) that selectively inhibits expression of a MYL2 transcript comprising a MYL2 polymorphism or mutation compared to a MYL2 transcript not comprising the MYL2 polymorphism or mutation (e.g., a polymorphism or mutation listed in FIG. 9 and/or FIG. 10). In some embodiments, the interfering RNA molecule inactivates the transcript comprising a MYL2 polymorphism or mutation at least 1.5 times, 2 times, 2.5 times or 3 times as much as it inactivates the transcript not comprising the MYL2 polymorphism or mutation. In some embodiments, the interfering RNA molecule comprises a nucleotide sequence that is complementary to a nucleotide sequence of the MYL2 transcript comprising the MYL2 polymorphism or mutation except for a single nucleotide mismatch at a position outside of the polymorphism or mutation. In some embodiments, the interfering RNA molecule comprises a nucleic acid sequence of 21 nucleotides in length, wherein 20 nucleotides of the nucleic acid sequence are complementary to the MYL2 transcript comprising the MYL2 polymorphism or mutation and no more than 19 nucleotides of the nucleic acid sequence are complementary to the transcript not comprising the MYL2 polymorphism or mutation.

In certain embodiments, provided herein is an interfering RNA molecule (e.g., a siRNA molecule or a shRNA molecule) that selectively inhibits expression a MYL3 transcript comprising a MYL3 polymorphism or mutation compared to a MYL3 transcript not comprising the MYL3 polymorphism or mutation (e.g., a polymorphism or mutation listed in FIG. 11 and/or FIG. 12). In some embodiments, the interfering RNA molecule inactivates the transcript comprising a MYL3 polymorphism or mutation at least 1.5 times, 2 times, 2.5 times or 3 times as much as it inactivates the transcript not comprising the MYL3 polymorphism or mutation. In some embodiments, the interfering RNA molecule comprises a nucleotide sequence that is complementary to a nucleotide sequence of the MYL3 transcript comprising the MYL3 polymorphism or mutation except for a single nucleotide mismatch at a position outside of the polymorphism or mutation. In some embodiments, the interfering RNA molecule comprises a nucleic acid sequence of 21 nucleotides in length, wherein 20 nucleotides of the nucleic acid sequence are complementary to the MYL3 transcript comprising the MYL3 polymorphism or mutation and no more than 19 nucleotides of the nucleic acid sequence are complementary to the transcript not comprising the MYL3 polymorphism or mutation.

In certain embodiments, provided herein is an interfering RNA molecule (e.g., a siRNA molecule or a shRNA molecule) that selectively inhibits expression of a TNNI3 transcript comprising a TNNI3 polymorphism or mutation compared to a TNNI3 transcript not comprising the TNNI3 polymorphism or mutation (e.g., a polymorphism or mutation listed in FIG. 13 and/or FIG. 14). In some embodiments, the interfering RNA molecule inactivates the transcript comprising a TNNI3 polymorphism or mutation at least 1.5 times, 2 times, 2.5 times or 3 times as much as it inactivates the transcript not comprising the TNNI3 polymorphism or mutation. In some embodiments, the interfering RNA molecule comprises a nucleotide sequence that is complementary to a nucleotide sequence of the TNNI3 transcript comprising the TNNI3 polymorphism or mutation except for a single nucleotide mismatch at a position outside of the polymorphism or mutation. In some embodiments, the interfering RNA molecule comprises a nucleic acid sequence of 21 nucleotides in length, wherein 20 nucleotides of the nucleic acid sequence are complementary to the TNNI3 transcript comprising the TNNI3 polymorphism or mutation and no more than 19 nucleotides of the nucleic acid sequence are complementary to the transcript not comprising the TNNI3 polymorphism or mutation.

In certain embodiments, provided herein is an interfering RNA molecule (e.g., a siRNA molecule or a shRNA molecule) that selectively inhibits expression of a TNNT2 transcript comprising a TNNT2 polymorphism or mutation compared to a TNNT2 transcript not comprising the TNNT2 polymorphism or mutation (e.g., a polymorphism or mutation listed in FIG. 15 and/or FIG. 16). In some embodiments, the interfering RNA molecule inactivates the transcript comprising a TNNT2 polymorphism or mutation at least 1.5 times, 2 times, 2.5 times or 3 times as much as it inactivates the transcript not comprising the TNNT2 polymorphism or mutation. In some embodiments, the interfering RNA molecule comprises a nucleotide sequence that is complementary to a nucleotide sequence of the TNNT2 transcript comprising the TNNT2 polymorphism or mutation except for a single nucleotide mismatch at a position outside of the polymorphism or mutation. In some embodiments, the interfering RNA molecule comprises a nucleic acid sequence of 21 nucleotides in length, wherein 20 nucleotides of the nucleic acid sequence are complementary to the TNNT2 transcript comprising the TNNT2 polymorphism or mutation and no more than 19 nucleotides of the nucleic acid sequence are complementary to the transcript not comprising the TNNT2 polymorphism or mutation.

In certain embodiments, provided herein is an interfering RNA molecule (e.g., a siRNA molecule or a shRNA molecule) that selectively inhibits expression of a TPM1 transcript comprising a TPM1 polymorphism or mutation compared to a TPM1 transcript not comprising the TPM1 polymorphism or mutation (e.g., a polymorphism or mutation listed in FIG. 17 and/or FIG. 18). In some embodiments, the interfering RNA molecule inactivates the transcript comprising a TPM1 polymorphism or mutation at least 1.5 times, 2 times, 2.5 times or 3 times as much as it inactivates the transcript not comprising the TPM1 polymorphism or mutation. In some embodiments, the interfering RNA molecule comprises a nucleotide sequence that is complementary to a nucleotide sequence of the TPM1 transcript comprising the TPM1 polymorphism or mutation except for a single nucleotide mismatch at a position outside of the polymorphism or mutation. In some embodiments, the interfering RNA molecule comprises a nucleic acid sequence of 21 nucleotides in length, wherein 20 nucleotides of the nucleic acid sequence are complementary to the TPM1 transcript comprising the TPM1 polymorphism or mutation and no more than 19 nucleotides of the nucleic acid sequence are complementary to the transcript not comprising the TPM1 polymorphism or mutation.

In certain embodiments, provided herein is an interfering RNA molecule (e.g., a siRNA molecule or a shRNA molecule) that selectively inhibits expression of a ACTC1 transcript comprising a ACTC1 polymorphism or mutation compared to a ACTC1 transcript not comprising the ACTC1 polymorphism or mutation (e.g., a polymorphism or mutation listed in FIG. 19 and/or FIG. 20). In some embodiments, the interfering RNA molecule inactivates the transcript comprising a ACTC1 polymorphism or mutation at least 1.5 times, 2 times, 2.5 times or 3 times as much as it inactivates the transcript not comprising the ACTC1 polymorphism or mutation. In some embodiments, the interfering RNA molecule comprises a nucleotide sequence that is complementary to a nucleotide sequence of the ACTC1 transcript comprising the ACTC1 polymorphism or mutation except for a single nucleotide mismatch at a position outside of the polymorphism or mutation. In some embodiments, the interfering RNA molecule comprises a nucleic acid sequence of 21 nucleotides in length, wherein 20 nucleotides of the nucleic acid sequence are complementary to the ACTC1 transcript comprising the ACTC1 polymorphism or mutation and no more than 19 nucleotides of the nucleic acid sequence are complementary to the transcript not comprising the ACTC1 polymorphism or mutation.

In some embodiments, provided herein is a nucleic acid molecule encoding an interfering RNA molecule disclosed or contemplated herein. In some embodiments, the nucleic acid molecule is a vector. In some embodiments, provided herein is a vector comprising a nucleic acid encoding an interfering RNA molecule disclosed or contemplated herein. In some embodiments, the vector has a tropism for cardiac tissue (e.g., an adeno-associated virus). In some embodiments, the inhibitory RNA molecule and/or the vector is operably linked to a cardiac-specific promoter (e.g. a cardiac specific troponin T promoter).

In some embodiments, provided herein are vector delivery systems that are capable of expressing the oligomeric, polymorphism or disease associated mutation-targeting sequences provided herein, such as vectors that express a polynucleotide sequence that express a polynucleotide sequence that is complementary to any or more of the target sequences provided in FIGS. 7-20. In certain embodiments, vector systems comprise vectors that express siRNA or other duplex-forming RNA interference molecules.

In some embodiments, provided herein is a kit comprising an interfering RNA molecule, a nucleic acid and/or a vector disclosed or contemplated herein. In some embodiments, the kit comprises at least 2, 3, 4, 5, 6, 7, 8, 9, 10, 11, 12, 13, 14, 15, 20, 25 or 30 different interfering RNA molecules disclosed or contemplated herein, wherein each different RNA molecule targets a different polymorphism or mutation on a gene selected from MYH7, MYBPC3, TNNT2, TNNI3, ACTC1, MYL2, MYL3 and TPM1. In some embodiments, the kit comprises at least 2, 3, 4, 5, 6, 7, 8, 9, 10, 11, 12, 13, 14, 15, 20, 25 or 30 nucleic acid molecules encoding different interfering RNA molecules disclosed or contemplated herein, wherein each different RNA molecule targets a different polymorphism or mutation on a gene selected from MYH7, MYBPC3, TNNT2, TNNI3, ACTC1, MYL2, MYL3 and TPM1. In some embodiments, the kit comprises at least 2, 3, 4, 5, 6, 7, 8, 9, 10, 11, 12, 13, 14, 15, 20, 25 or 30 vectors encoding different interfering RNA molecules disclosed or contemplated herein, wherein each different RNA molecule targets a different polymorphism or mutation on a gene selected from MYH7, MYBPC3, TNNT2, TNNI3, ACTC1, MYL2, MYL3 and TPM1.

### BRIEF DESCRIPTION OF THE DRAWINGS

FIG. 1 shows selective silencing of Myh6 R403Q expression by AAV-9-mediated RNAi. (A) Schematic representation of FVB, 129SvEv, 129SvEv mutant (R403Q) transcript and RNAi sequences. (B) Quantitative real-time PCR analysis of wild-type Myh6 (white bar) and mutant Myh6 R403Q (black bar) expression after transduction of the 403m and 403i constructs (n=4). Levels of the transcripts were normalized to control LacZ RNAi. (C) Quantitative real-time PCR analysis of FVB Myh6 (white bar) and 129SvEv Myh6 R403Q (black bar) expression after transduction of the 129i construct (n=4). Levels of the transcripts were normalized to control LacZ RNAi. Data are presented as the mean±s.d.

FIG. 2 shows an in vivo effect of Myh6 R403Q silencing. (A) Cardiac histopathology from MHC mice transduced with control RNAi (left) and 403i RNAi (right). Masson trichrome staining reveals marked fibrosis in MHC^{403/+} mice transduced with control RNAi. Bar=1 mm. (B) Hematoxylin and eosin staining shows myocyte disarray in MHC^{403/+} mice transduced with control RNAi (left) and normal myocyte architecture in mice transduced with 403i RNAi (right). Bar=100 μm. (C) Quantification of myocardial fibrosis in MHC^{403/+} mice transduced with control RNAi (black bar, n=4) and 403i RNAi (white bar, n=4). (D) An electrocardiogram of MHC^{403/+} mice transduced with control RNAi (left top panel) and 403i RNAi (right top panel). Mice transduced with control RNAi have prolonged QRS (ventricular conduction) interval and high voltage P waves consistent with LV hypertrophy and atrial enlargement. The bottom panel presents QRS intervals from mice transduce with control RNAi (black bar, n=5) and 403i RNAi (white bar, n=6). (E) Quantitative real-time PCR analysis of Nppa (left panel) and Nppb (right panel) expression after transduction of control RNAi (black bar) and two different doses of 403i constructs (white bar) (n=5). Levels of the transcripts were normalized to transcript levels from age matched wild-type hearts. Data are presented as the mean±s.d.

FIG. 3 (a) shows a schematic representation of mutant (R403Q) transcript and RNAi sequences. (b) Shows a schematic representation of AAV vector including cTnT, cardiac troponin promoter; EGFP, enhanced green fluorescent protein; and RNAi, RNAi cassette. (c) Shows the relative expression of mutant Myh6 R403Q to wild-type transcripts (quantified by RNAseq) in hearts from 14-day old MHC^{403/+} mice injected with AAV-9-control virus or AAV9-403i virus at neonatal day 0. Neonatal MHC^{403/+} mice were injected with different amounts of virus (vg/kg).

FIG. 4 shows AAV-9 and cTnT promote selective expression of EGFP in the heart. GFP signals were visualized with fluorescence (left) and light (right) microscopy to assess expression in organs isolated from mice at three weeks (a) or three months (b) after transduction with AAV-9 encoding EGFP under the control of the cTnT-promoter.

FIG. 5 shows confocal micrographs of cardiac sections from mice age 5 months (a) and 12 months (b) after RNAi transduction at day one of life demonstrate long-term EGFP expression in myocytes. DAPI (blue), GFP (green), troponin I (red). Bar=40 μm.

FIG. 6 shows schematics of RNAi silencing protocols. (a) MHC^{403/+} mice transduced with RNAi on day 1 of life and subsequent treated with CsA to accelerate hypertrophic remodeling from age 5 weeks through age 8 weeks, at which time cardiac evaluations were performed. (b) MHC^{403/+} mice were transduced with RNAi (5×10¹² vg/kg or 5×10¹³ vg/kg) on day 21 of life and then treated with CsA from ages 7 weeks through age 10 weeks at which time cardiac evaluations were performed. (c) MHC^{403/+} mice were treated with CsA for 3 weeks, beginning 21 day of life. At 6 weeks of age, mice were transduced with RNAi and cardiac evaluations were performed at age 14 weeks.

FIG. 7 is a table that lists exemplary polymorphisms and mutations found in human MYH7. The location of the polymorphism is with reference to the translational start site, with the “A” in the “ATG” start codon being position 1.

FIG. 8 is a table that lists exemplary disease associated mutations found in human MYH7. DCM refers to dilated cardiomyopathy. HCM refers to hypertrophic cardiomyopathy. LVNC refers to left ventricular non-compaction. The location of the mutation is with reference to the translational start site, with the “A” in the “ATG” start codon being position 1.

FIG. 9 is a table that lists exemplary polymorphisms and mutations found in human MYL2. The location of the polymorphism/mutation is with reference to the translational start site, with the “A” in the “ATG” start codon being position 1.

FIG. 10 is a table that lists exemplary disease associated mutations found in human MYL2. HCM refers to hypertrophic cardiomyopathy. The location of the mutation is with reference to the translational start site, with the “A” in the “ATG” start codon being position 1.

FIG. 11 is a table that lists exemplary polymorphisms and mutations found in human MYL3. The location of the polymorphism is with reference to the translational start site, with the “A” in the “ATG” start codon being position 1.

FIG. 12 is a table that lists exemplary disease associated mutations found in human MYL3. HCM refers to hypertrophic cardiomyopathy. The location of the mutation is with reference to the translational start site, with the “A” in the “ATG” start codon being position 1.

FIG. 13 is a table that lists exemplary polymorphisms and mutations found in human TNNI3. The location of the polymorphism is with reference to the translational start site, with the “A” in the “ATG” start codon being position 1.

FIG. 14 is a table that lists exemplary disease associated mutations found in human TNNI3. DCM refers to dilated cardiomyopathy. HCM refers to hypertrophic cardiomyopathy. The location of the mutation is with reference to the translational start site, with the “A” in the “ATG” start codon being position 1.

FIG. 15 is a table that lists exemplary polymorphisms and mutations found in human TNNT2. The location of the polymorphism is with reference to the translational start site, with the “A” in the “ATG” start codon being position 1.

FIG. 16 is a table that lists exemplary disease associated mutations found in human TNNT2. DCM refers to dilated cardiomyopathy. HCM refers to hypertrophic cardiomyopathy. The location of the mutation is with reference to the translational start site, with the “A” in the “ATG” start codon being position 1.

FIG. 17 is a table that lists exemplary polymorphisms and mutations found in human TPM1. The location of the polymorphism is with reference to the translational start site, with the “A” in the “ATG” start codon being position 1.

FIG. 18 is a table that lists exemplary disease associated mutations found in human TPM1. DCM refers to dilated cardiomyopathy. HCM refers to hypertrophic cardiomyopathy. The location of the mutation is with reference to the translational start site, with the “A” in the “ATG” start codon being position 1.

FIG. 19 is a table that lists exemplary polymorphisms and mutations found in human ACTC1. The location of the polymorphism is with reference to the translational start site, with the “A” in the “ATG” start codon being position 1.

FIG. 20 is a table that lists exemplary disease associated mutations found in human ACTC1. DCM refers to dilated cardiomyopathy. HCM refers to hypertrophic cardiomyopathy. The location of the mutation is with reference to the translational start site, with the “A” in the “ATG” start codon being position 1.

FIG. 21 provides reference transcript sequences for HCM associated genes.

FIG. 22 is a table that shows RNAi effects on cardiac morphology and function in HCM mice. To accelerate hypertrophic remodeling in MHC^{403/+} mice CsA was administered for the number of weeks indicated either after (Post) RNAi transduction on day 1 or for 3 weeks prior (Pre) to RNAi transduction on day 21. No CsA denotes MHC^{403/+} mice not treated with CsA. Age, denotes age at time of cardiac evaluation; (#) number of mice studies. LVDD, LV diastolic dimensions; LVWT, LV wall thickness; FS, percent fractional shortening. Cardiac dimensions and function with associated P values, calculated by T-test, reflect comparisons to MHC^{403/+} transduced with control RNAi. Values for wildtype 129SvEv mice, not treated with CsA, are shown for comparison. Data are presented as the mean±s.d.

FIG. 23 is a table that shows viral dosage need for RNAi effects on cardiac morphology and function in HCM mice. MHC^{403/+} mice (n=number) were transduced with RNAi at vector genomes per kg (titer) per kg on day 1 and treated with CsA for 3 weeks to accelerate hypertrophic remodeling. LVDD (LV diastolic dimension; LVWT, LV wall thickness; FS %, percent fractional shortening and left atria (LA) dimension normalized to the aortic root (Ao) are provide. Cardiac dimensions and function with associated P values, calculated by T-test, reflect comparisons to MHC^{403/+} transduced with control RNAi. Data are presented as the mean±s.d.

### DETAILED DESCRIPTION

### General

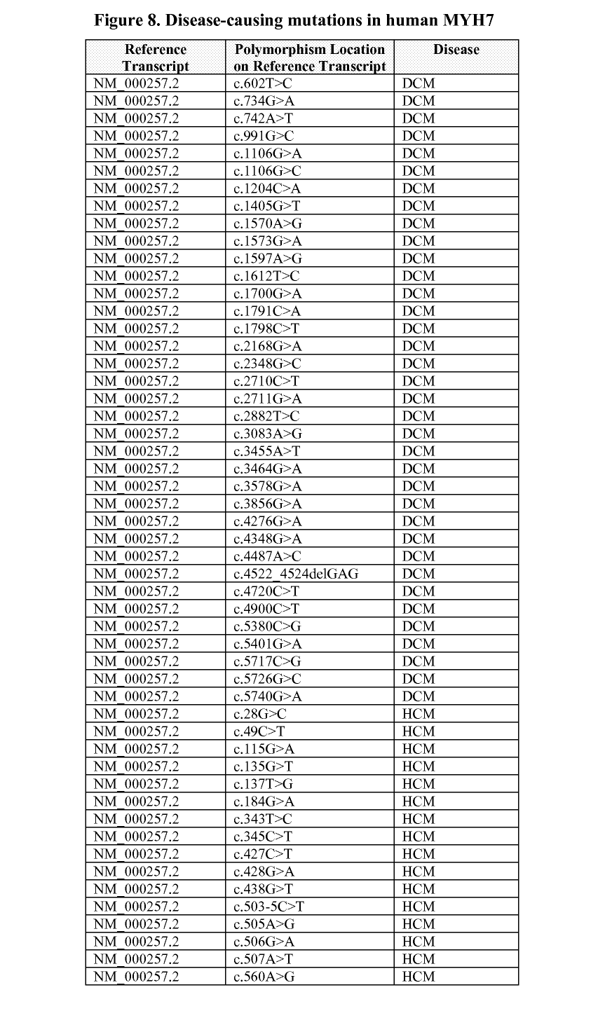

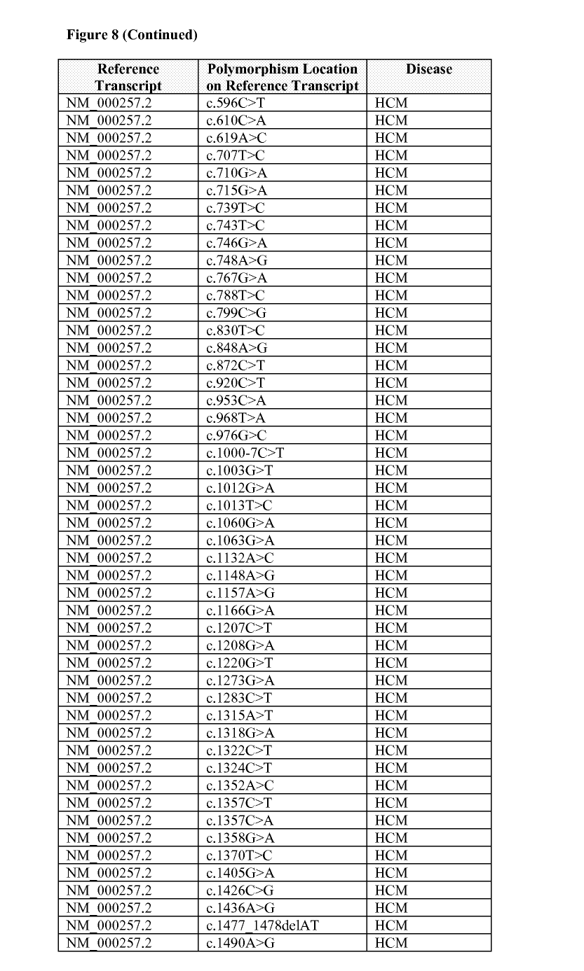

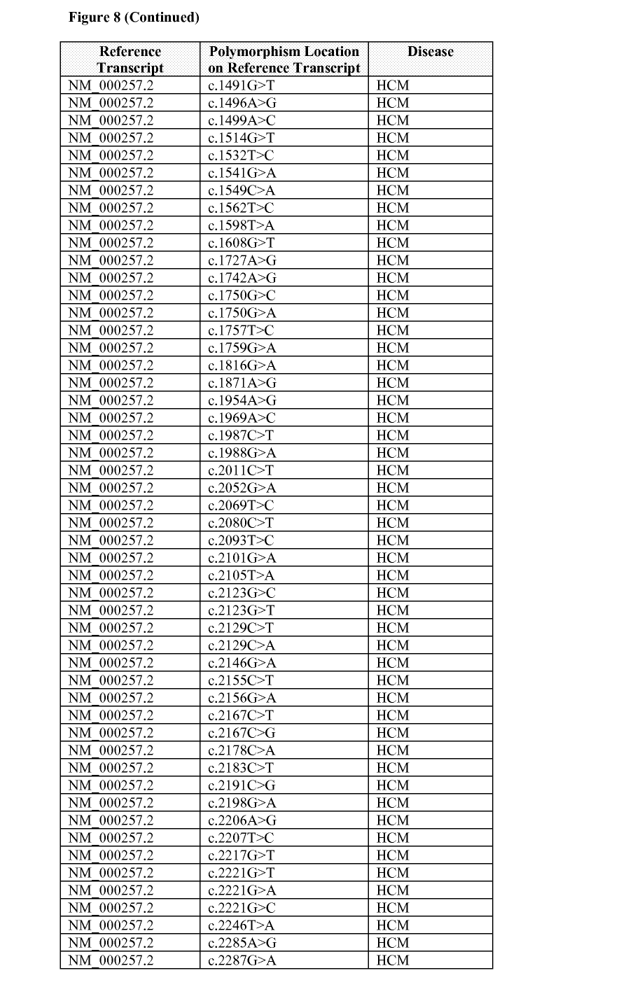

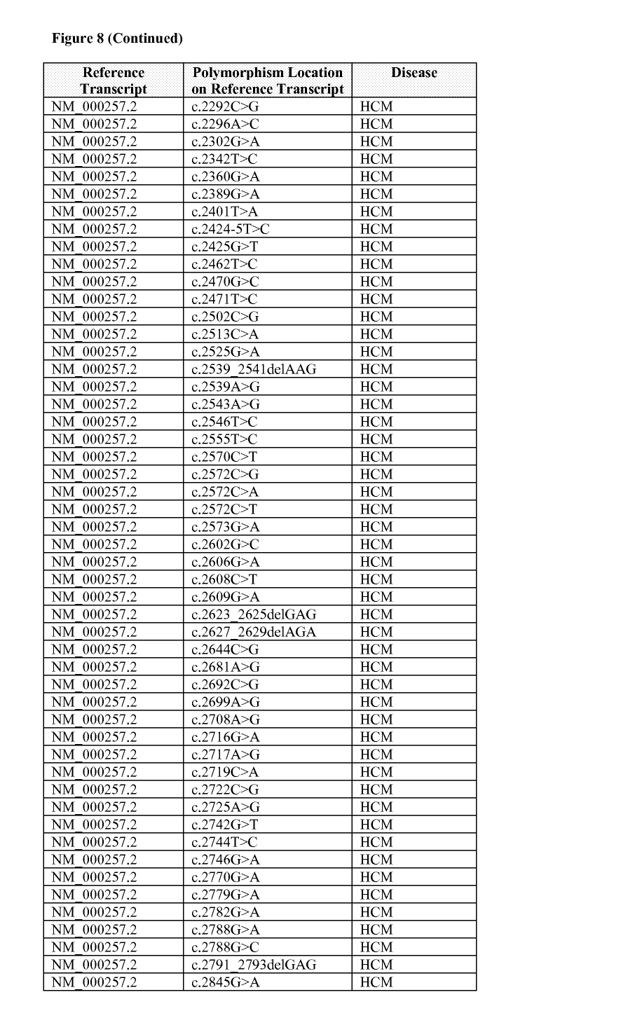

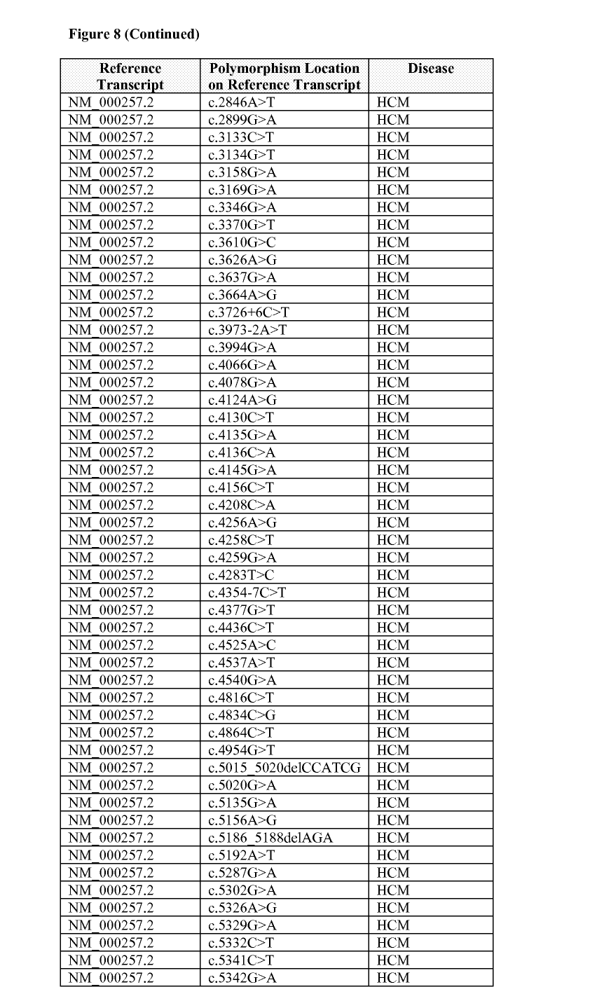

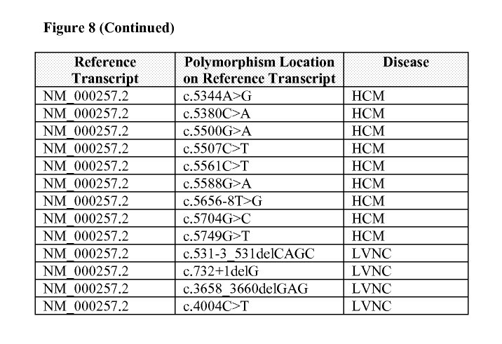

Provided herein are methods and compositions useful for the treatment of hypertrophic cardiomyopathy (HCM), dilated cardiomyopathy (DCM) and/or left ventricular non-compaction (LVNC) through allele-specific RNA silencing. Mutations found in eight genes (MYH7, MYBPC3, TNNT2, TNNI3, ACTC1, MYL2, MYL3 and TPM1) cause hypertrophic cardiomyopathy (HCM), dilated cardiomyopathy (DCM) and/or left ventricular non-compaction (LVNC). Mutations found in 7 of these genes (all except MYBPC3) are dominant negative mutations that result in the production of a “poison polypeptide” that causes the disease phenotype. Such mutations are provided in FIGS. 8, 10, 12, 14, 16, 18 and 20. As disclosed or contemplated herein, selectively blocking the production of the “poison polypeptide” by preventing translation of RNA transcripts containing the disease-causing mutation (e.g., by destroying such transcripts) is an effective method for the treatment and/or prevention of HCM, DCM and LVNC. Provided herein are compositions and methods for the selective inhibition of the translation of transcripts harbouring disease-causing mutations compared to transcripts that do not contain disease-causing mutations through the use of allele-specific antisense oligonucleotides, including interfering RNA molecules.

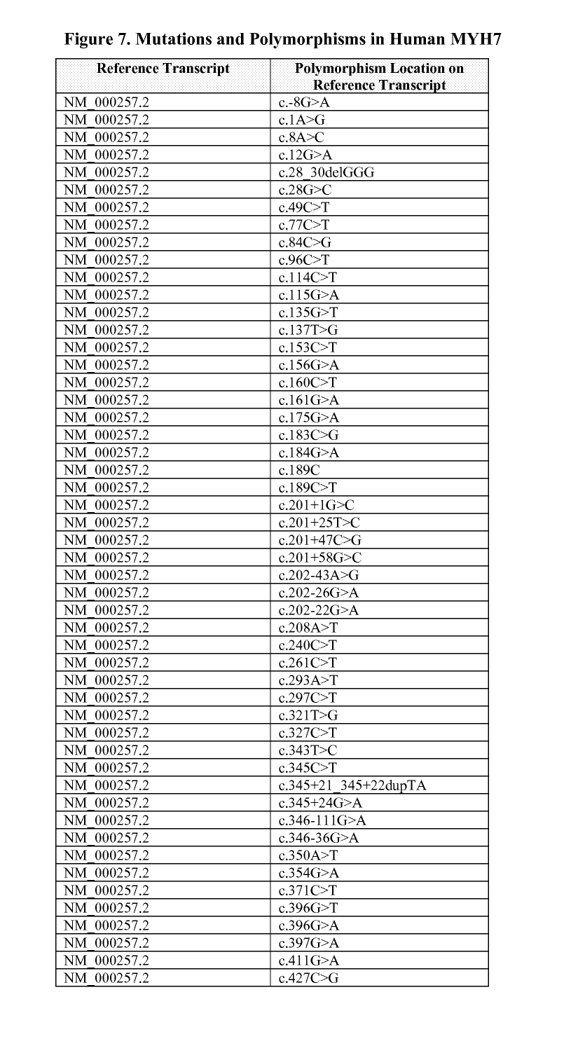

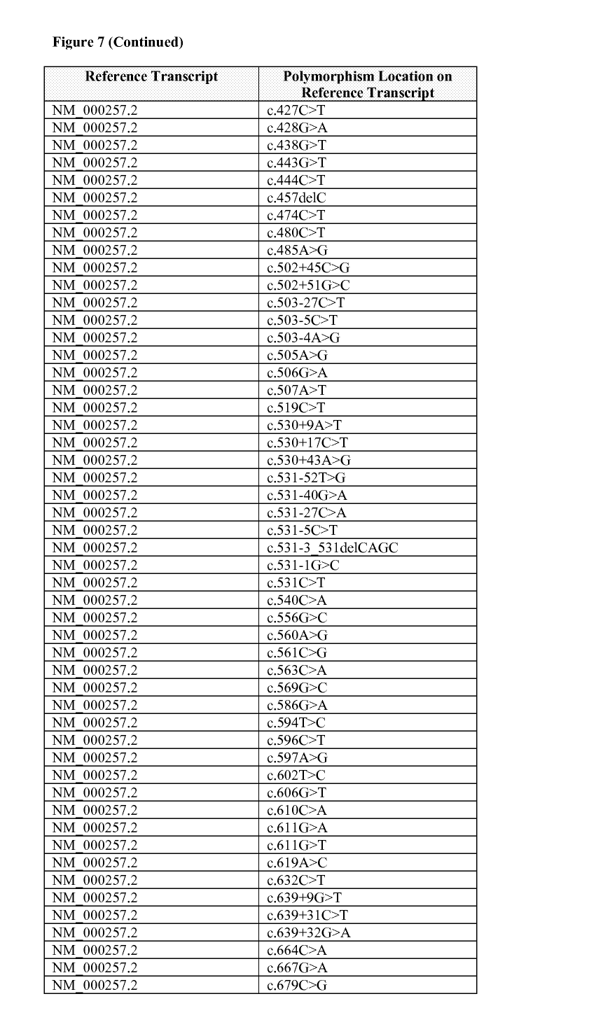

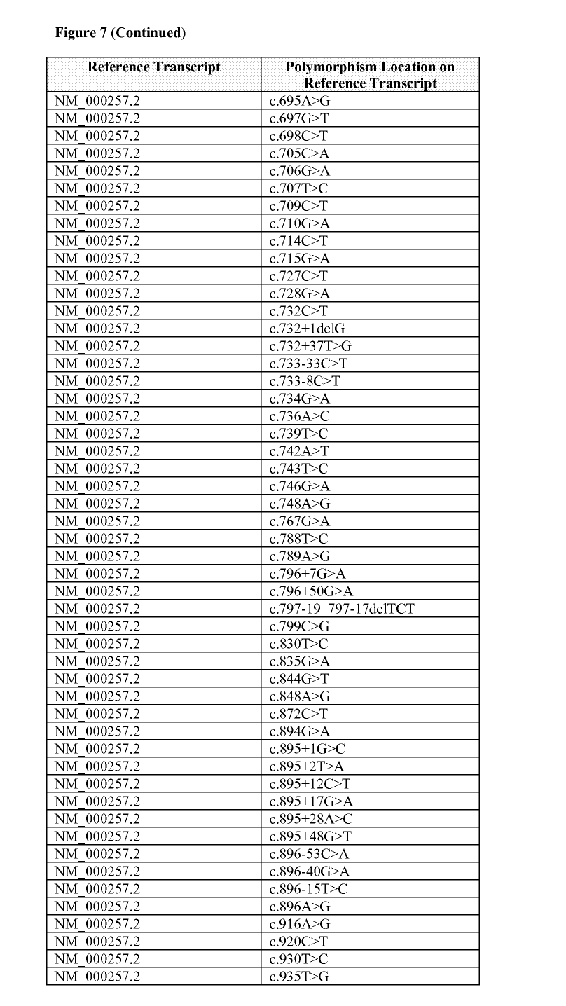

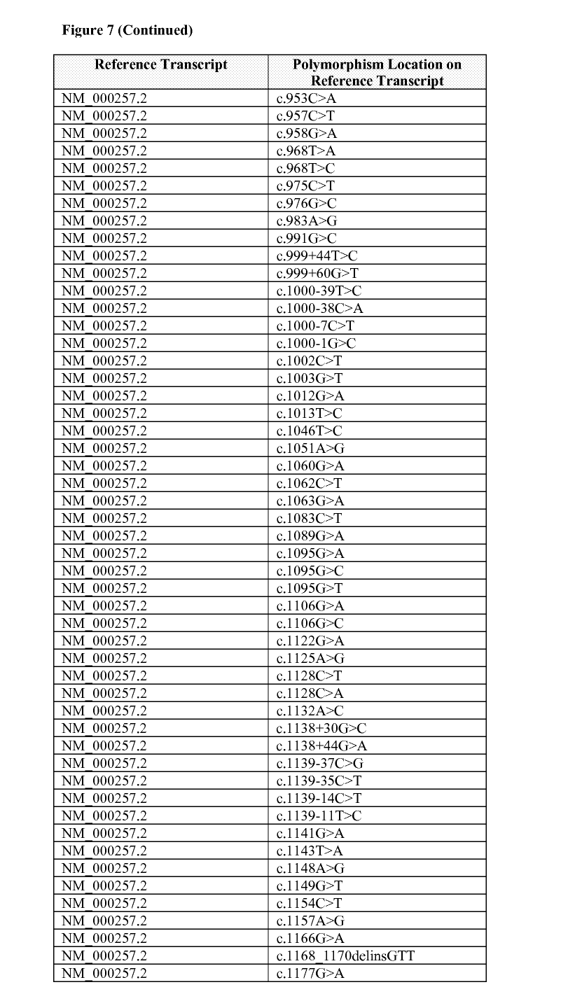

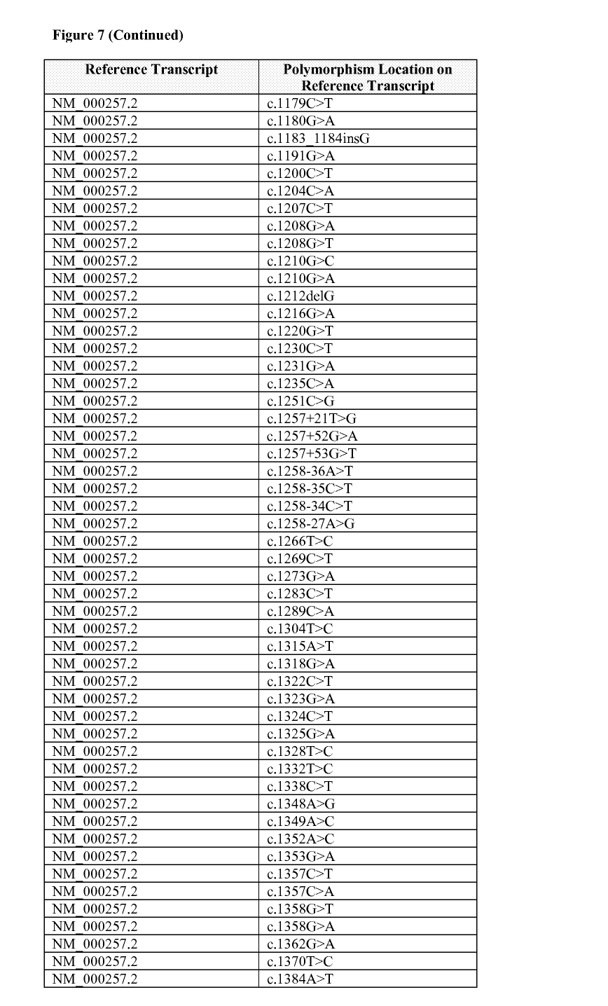

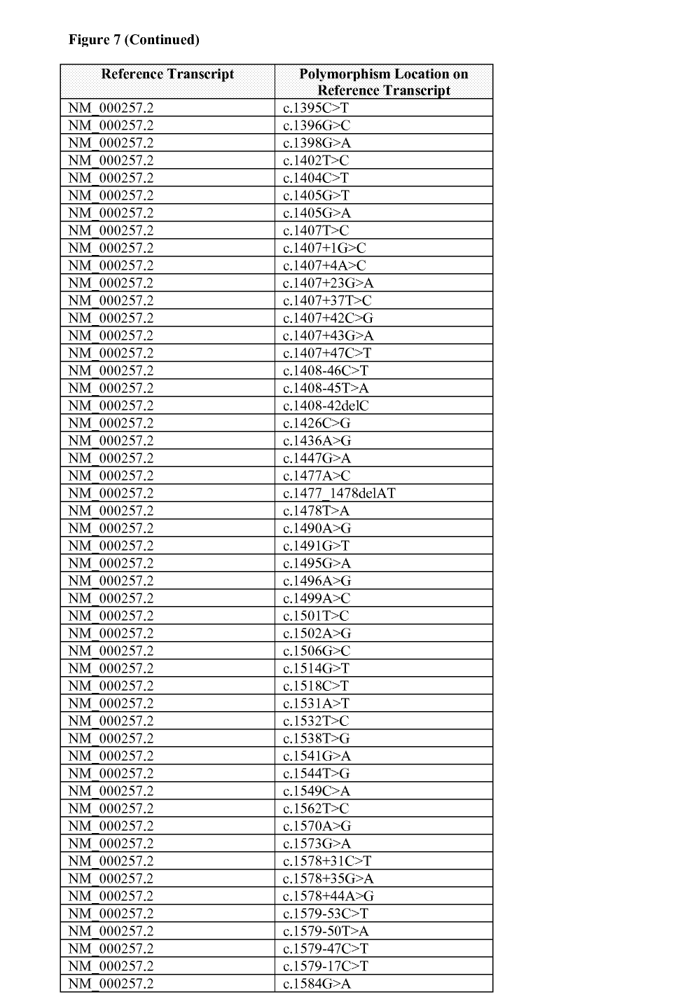

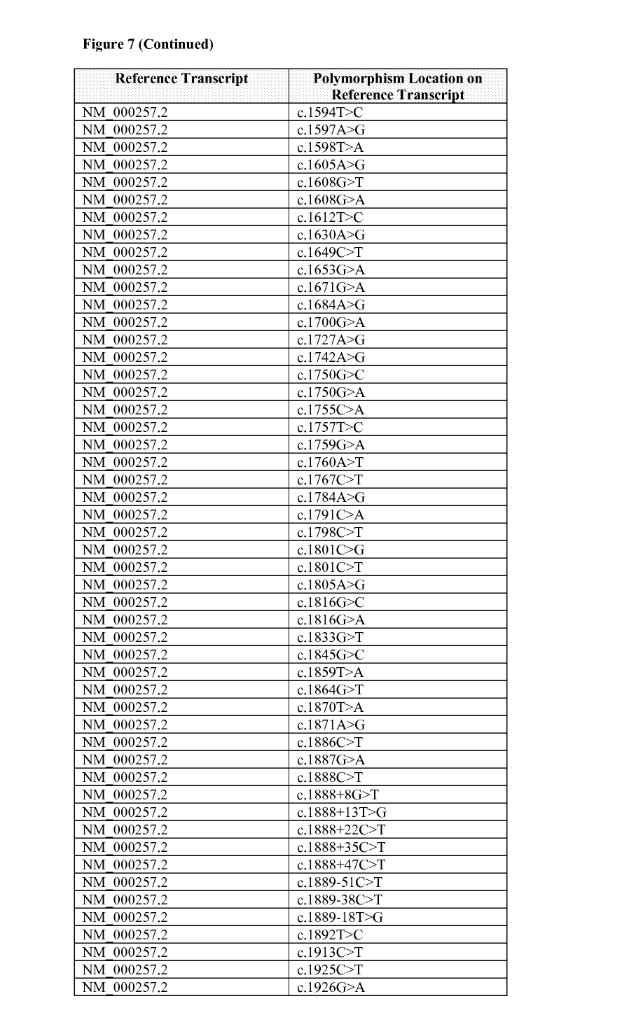

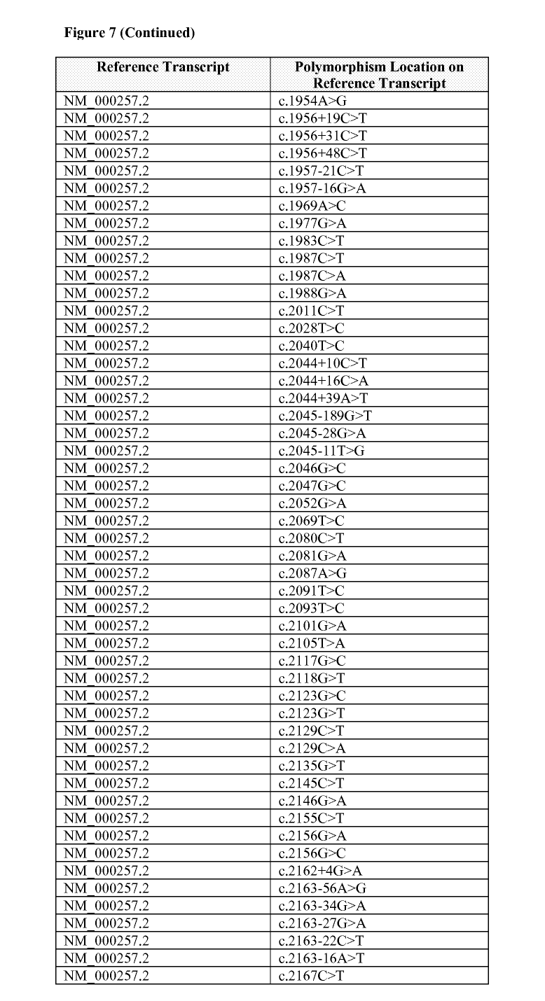

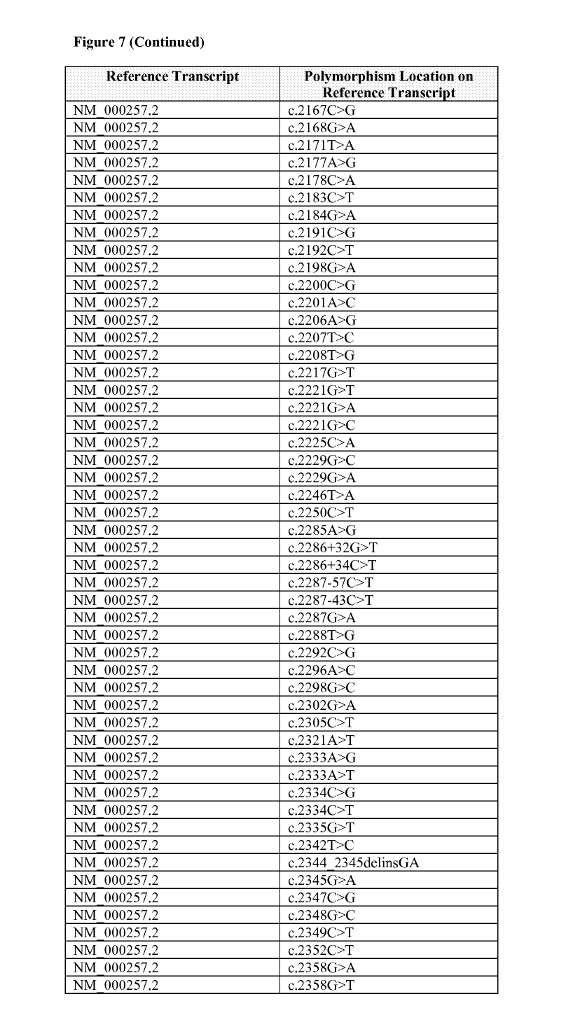

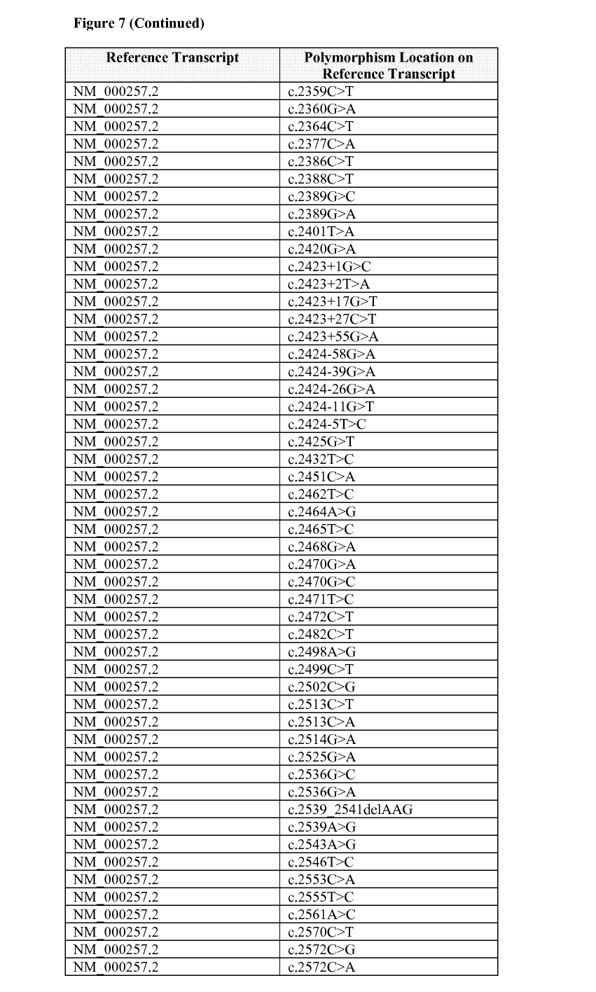

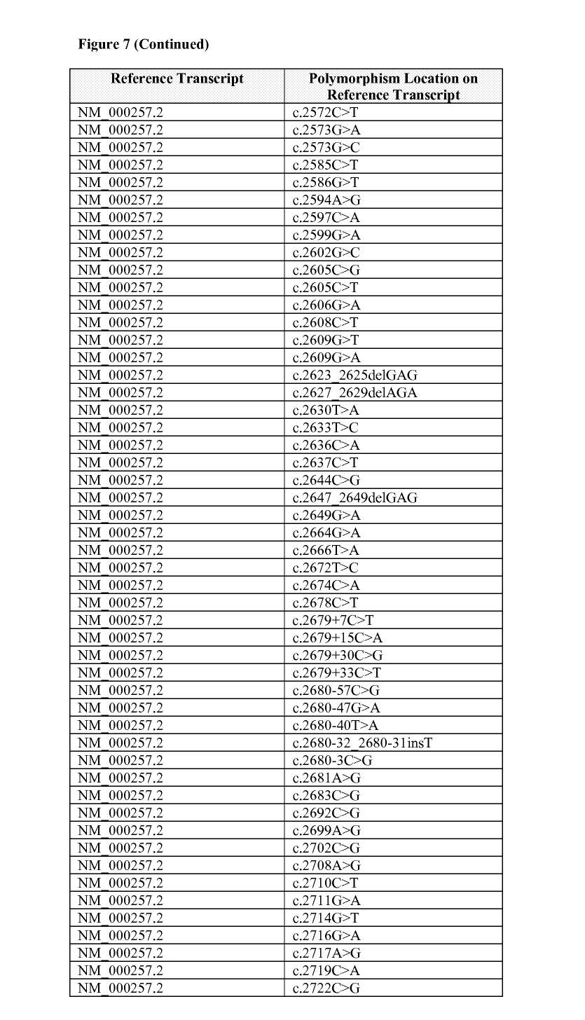

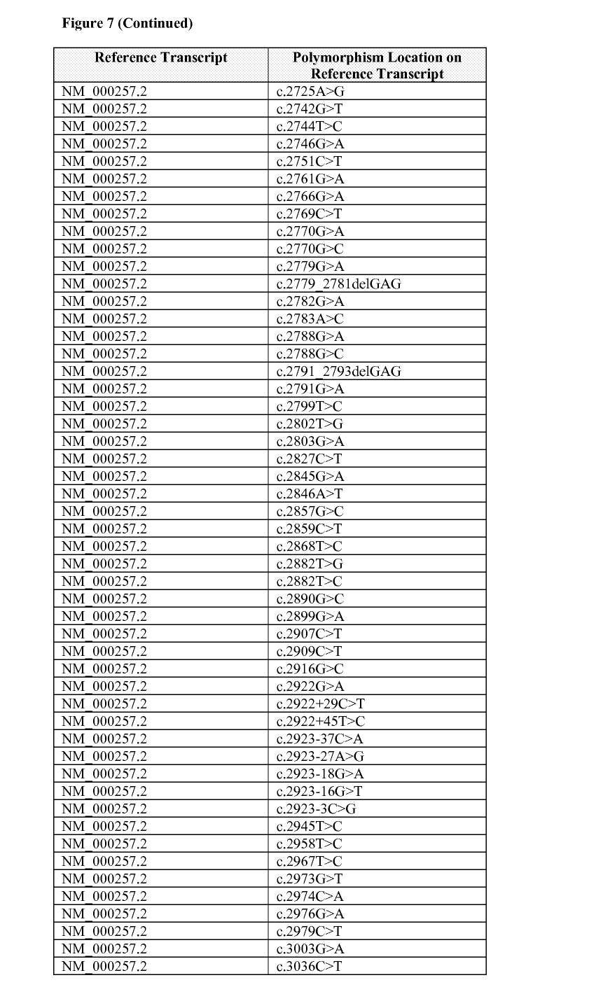

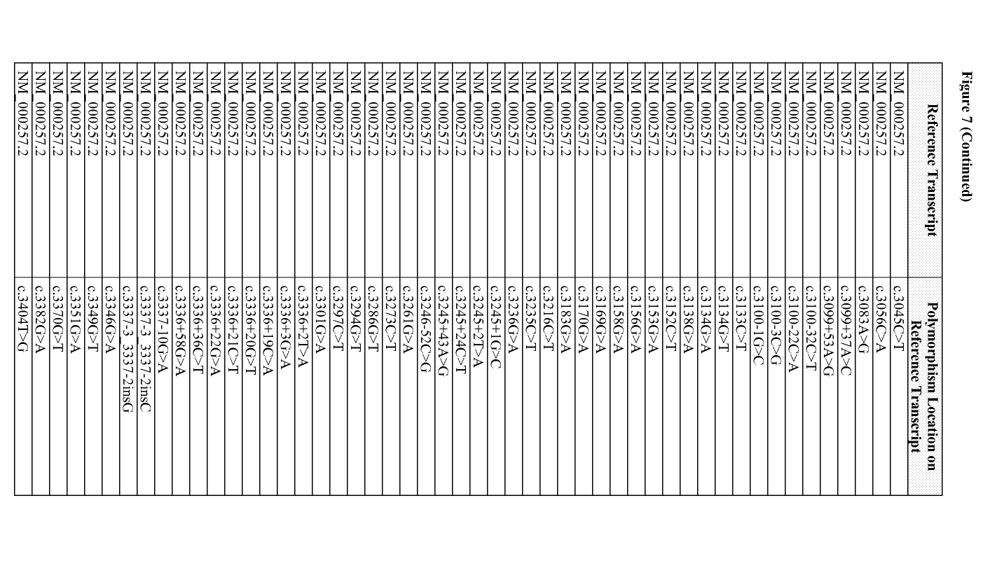

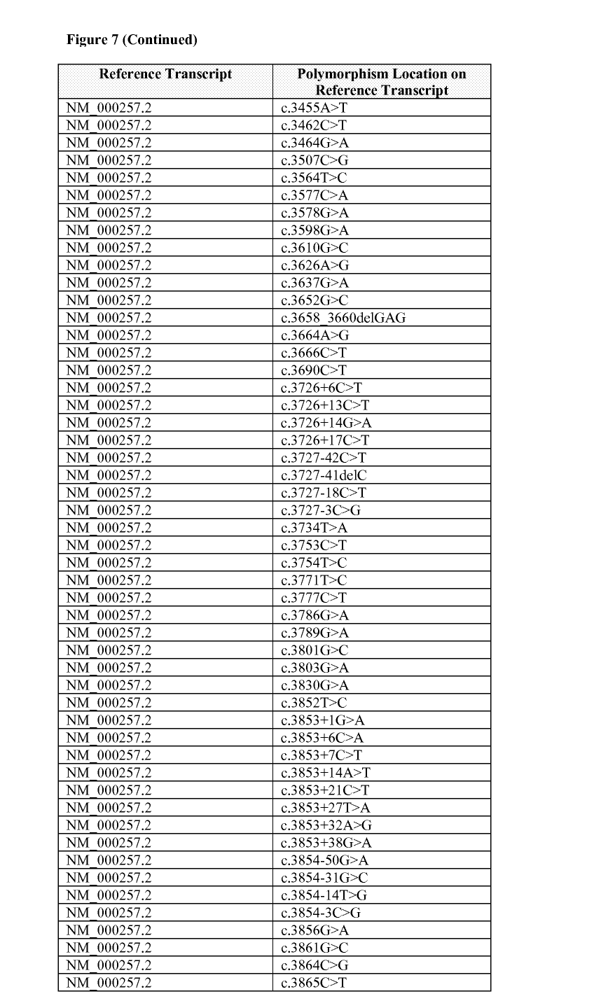

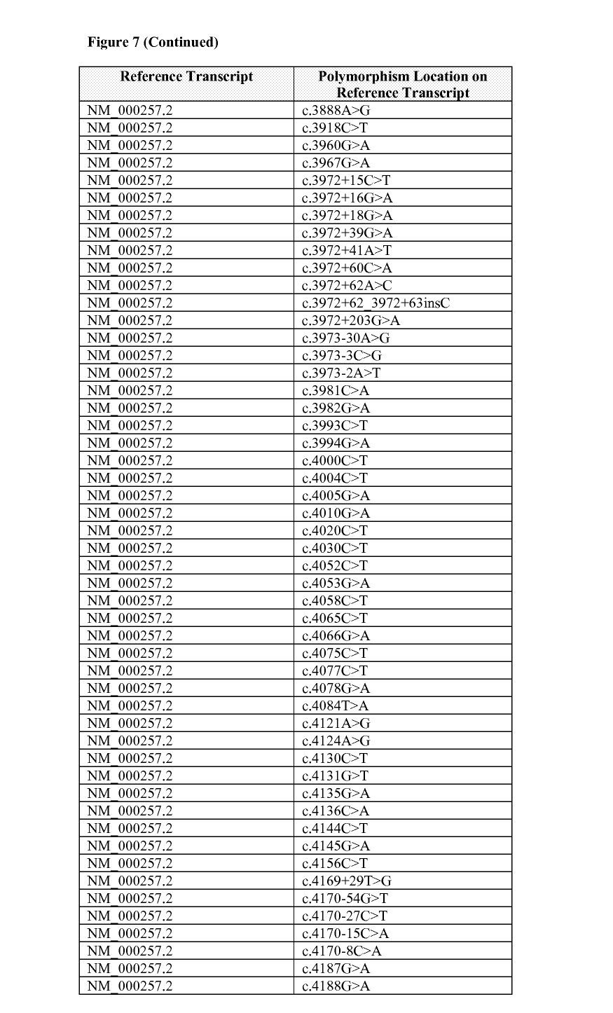

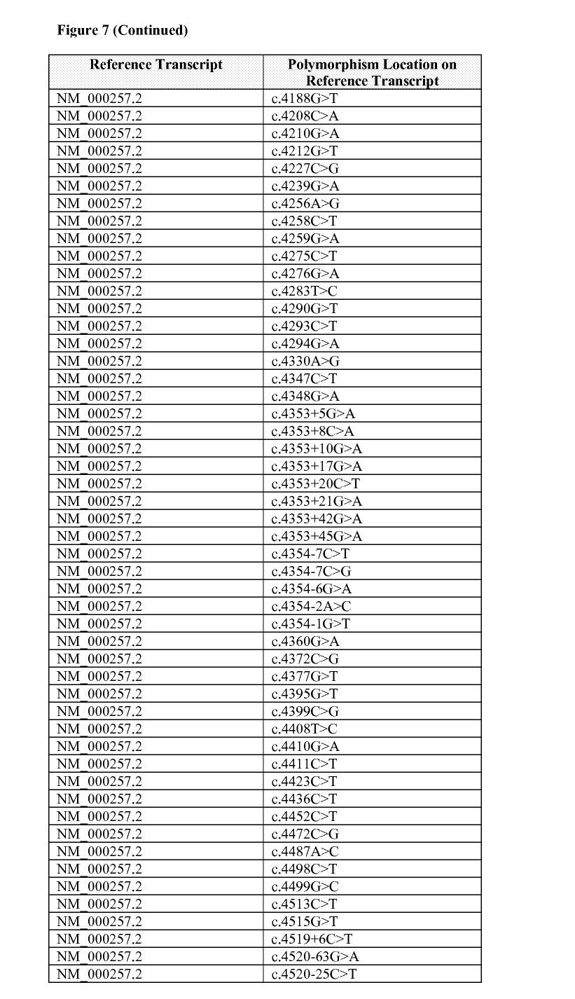

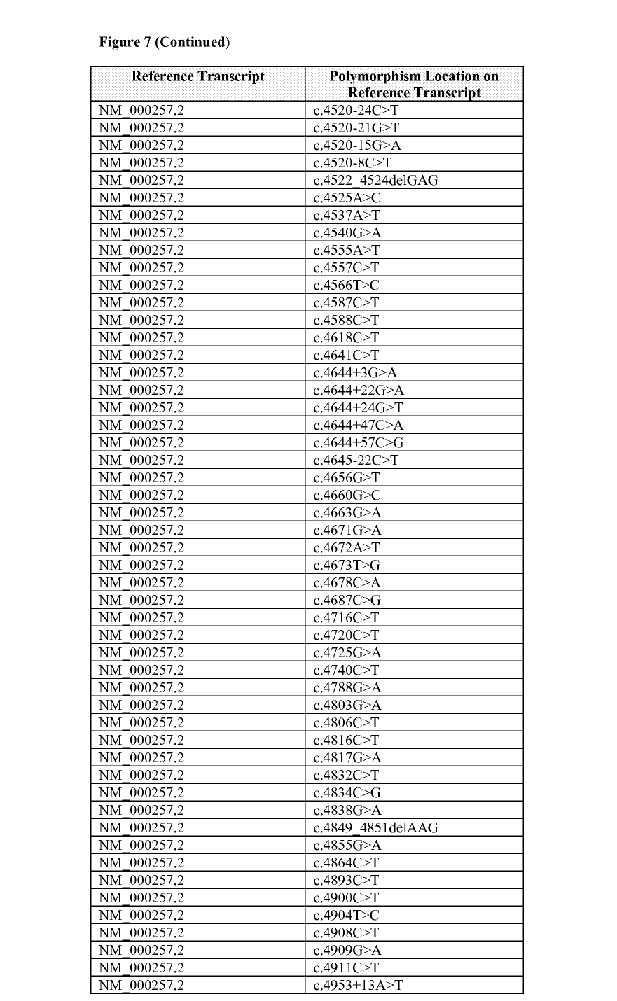

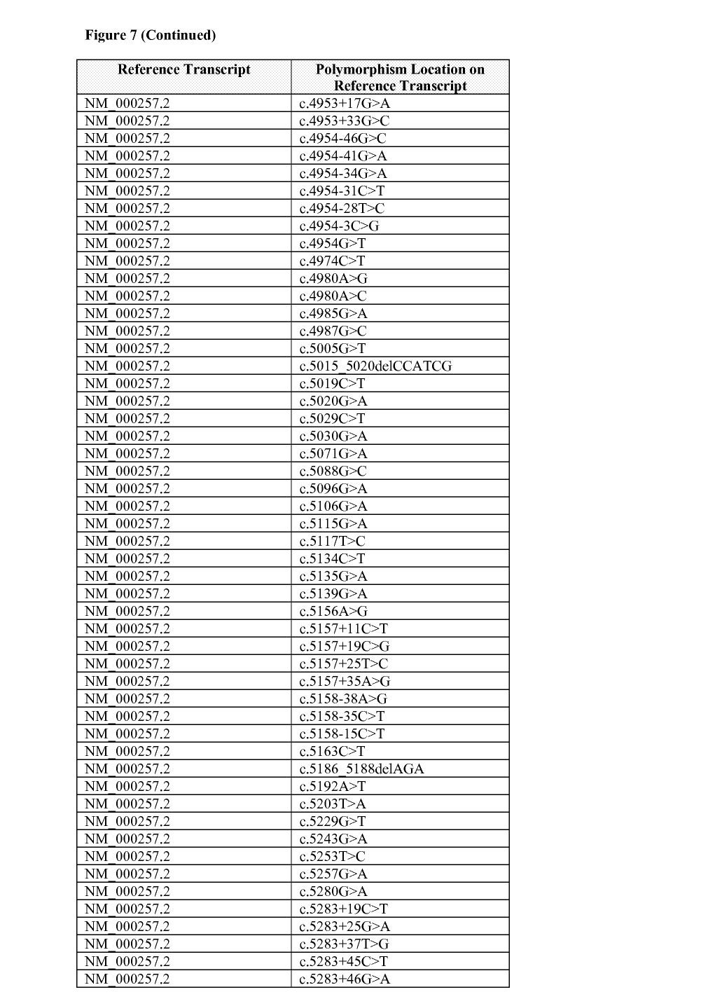

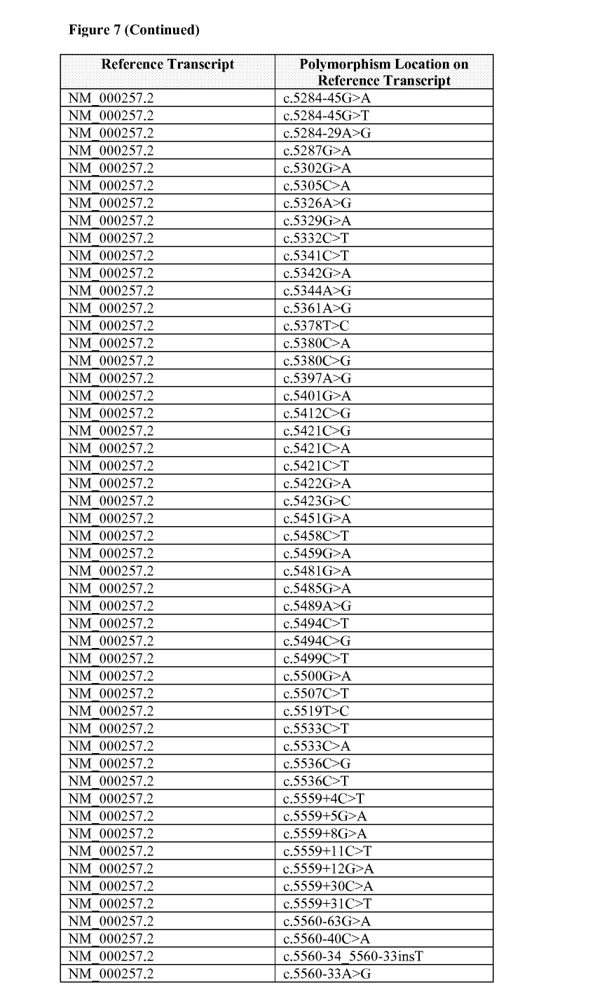

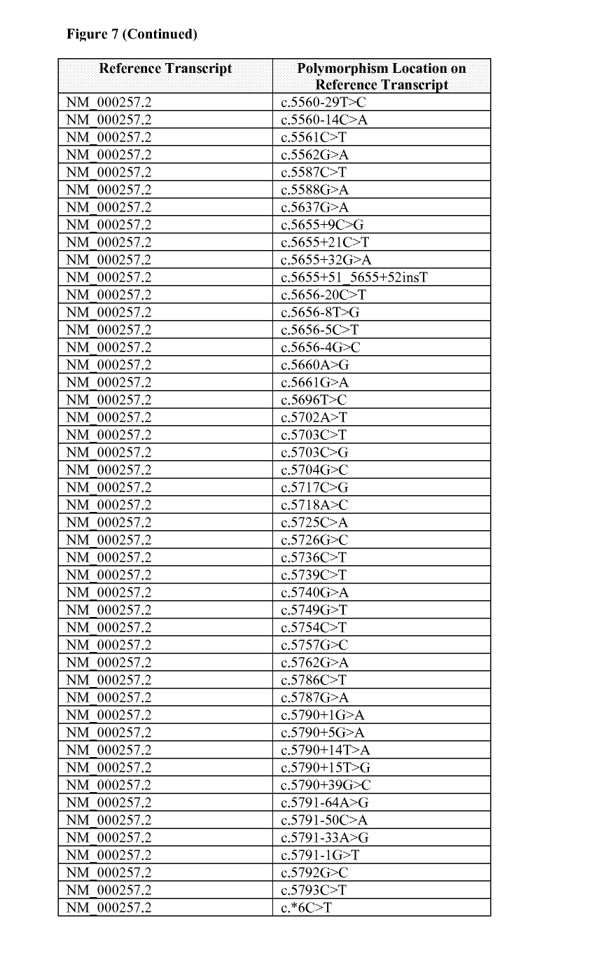

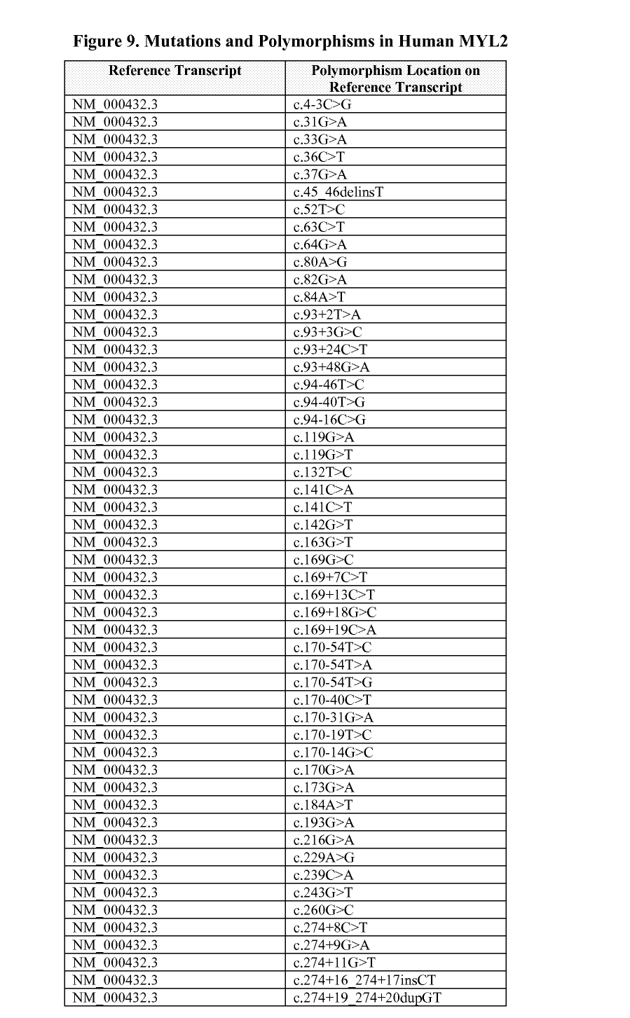

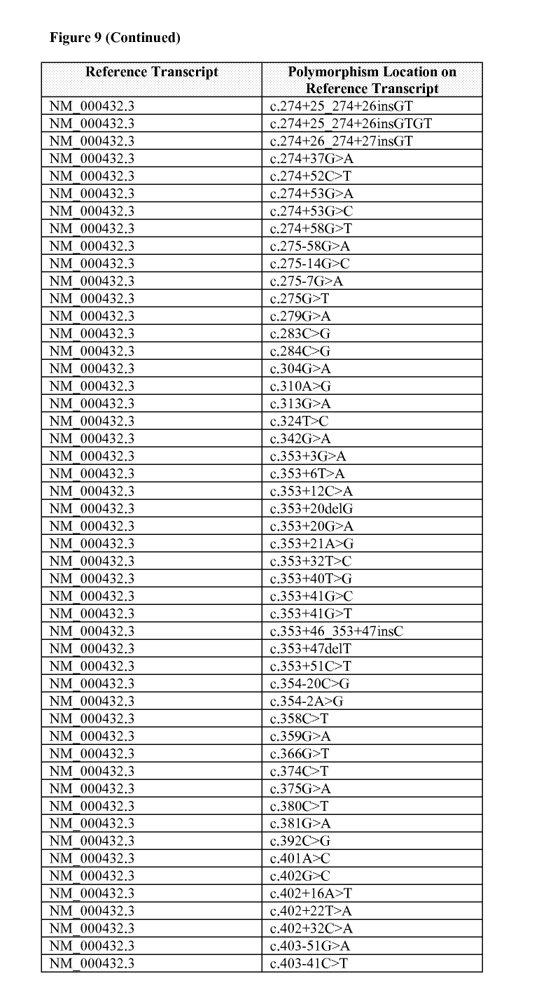

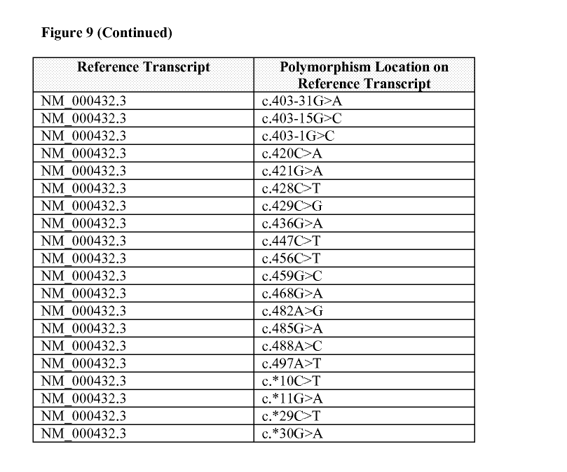

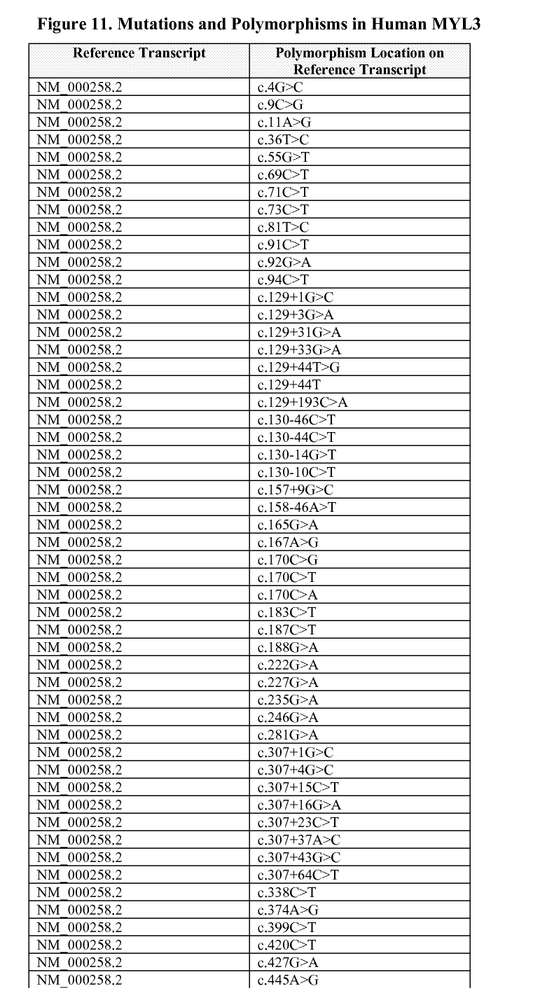

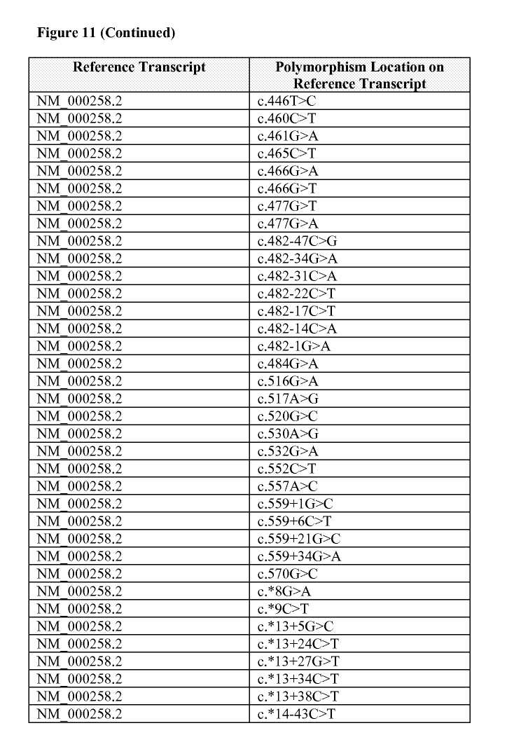

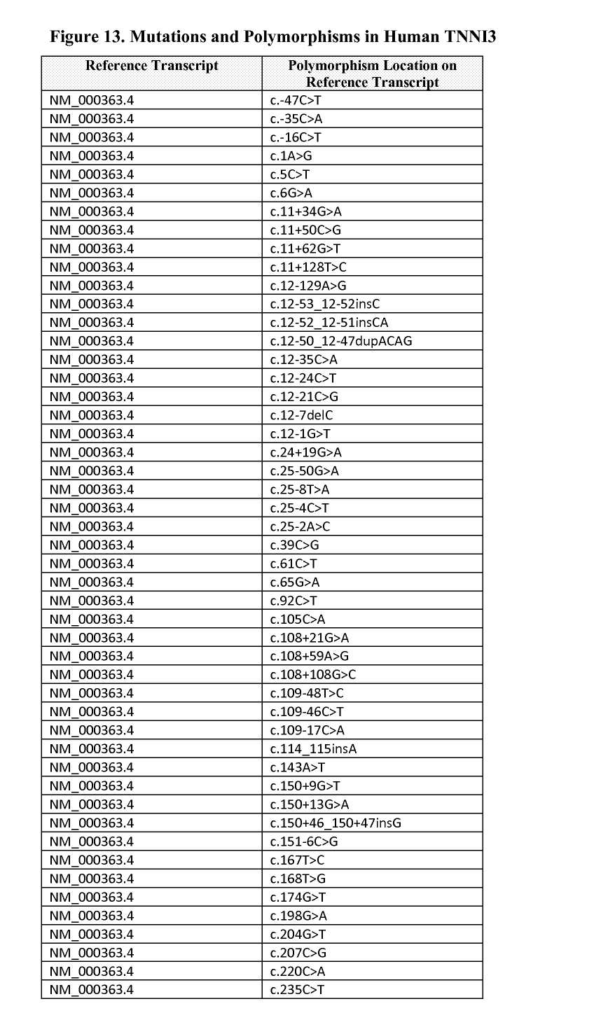

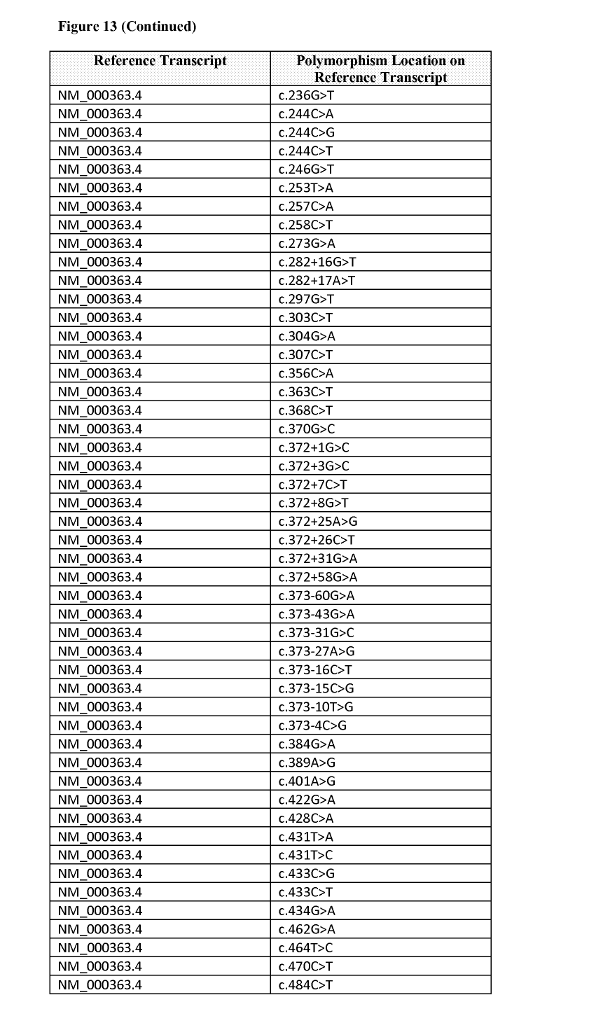

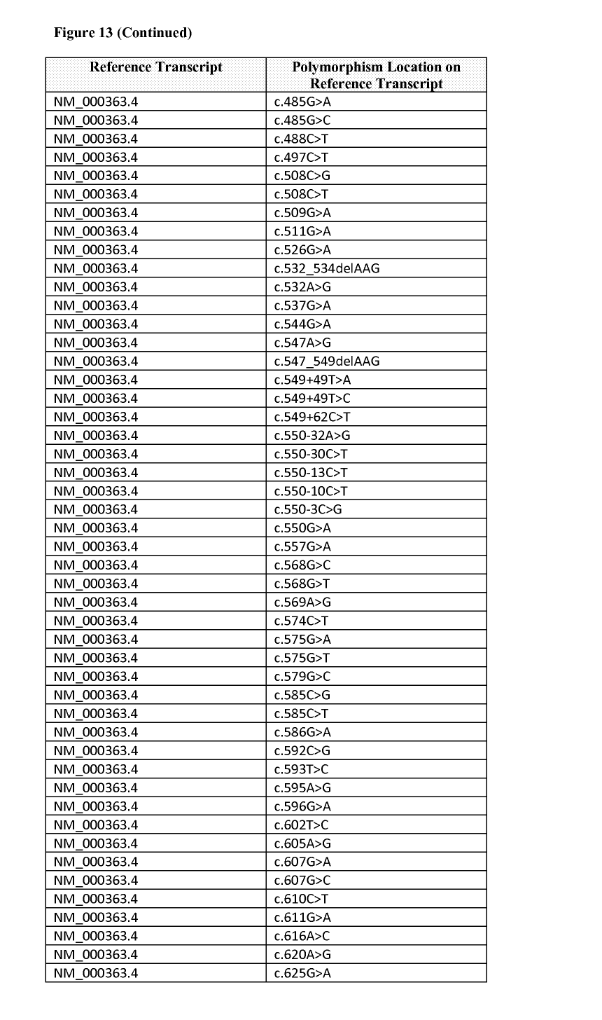

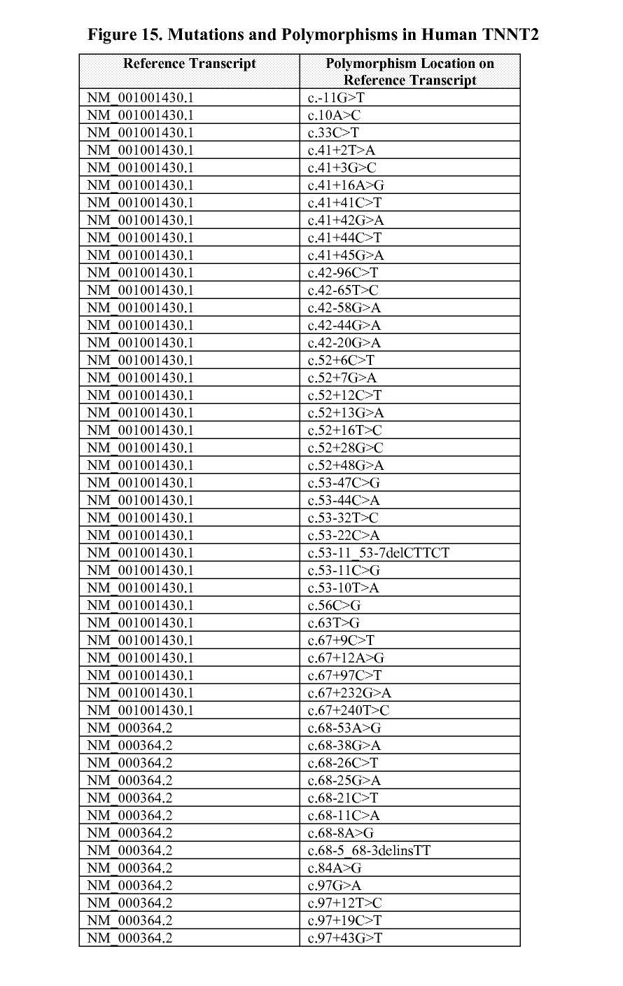

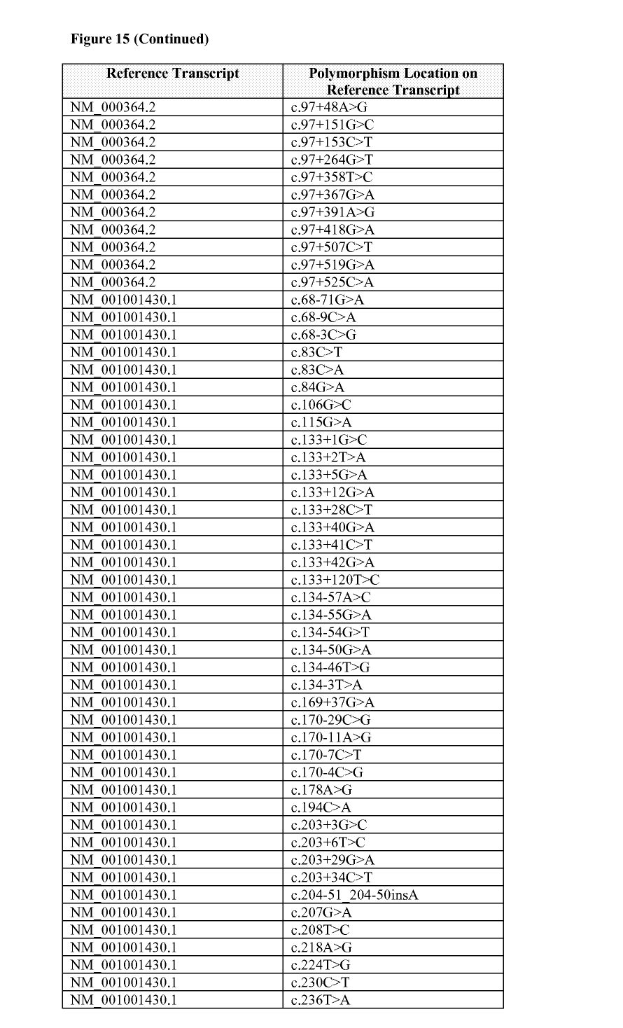

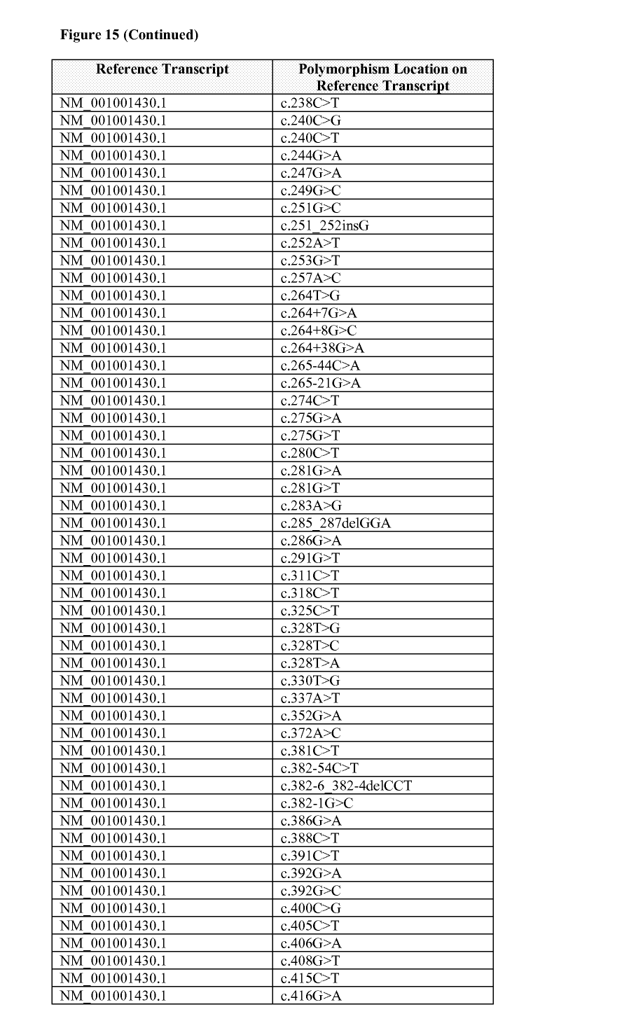

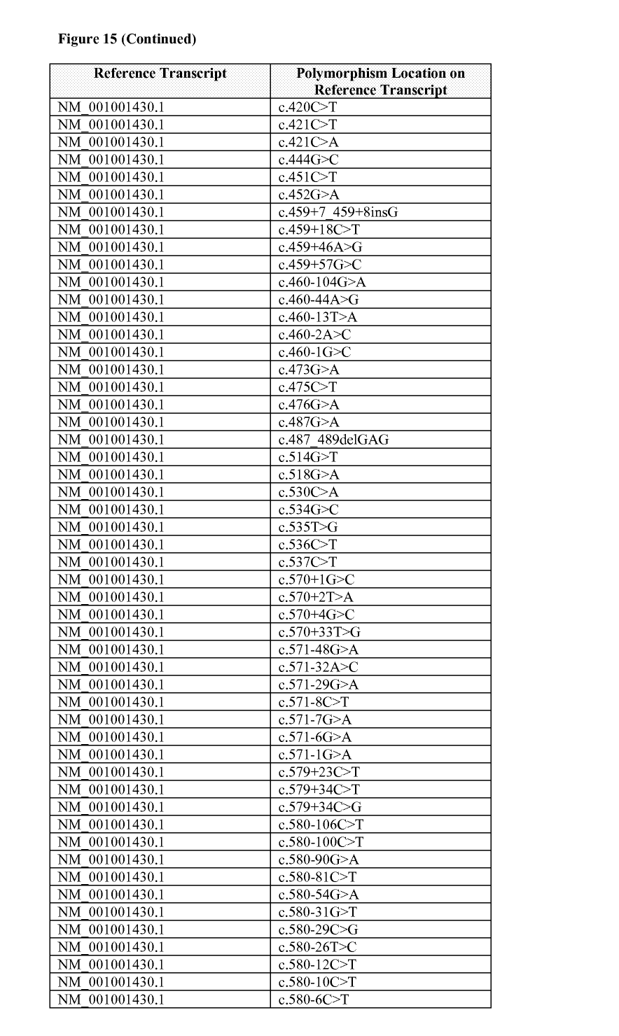

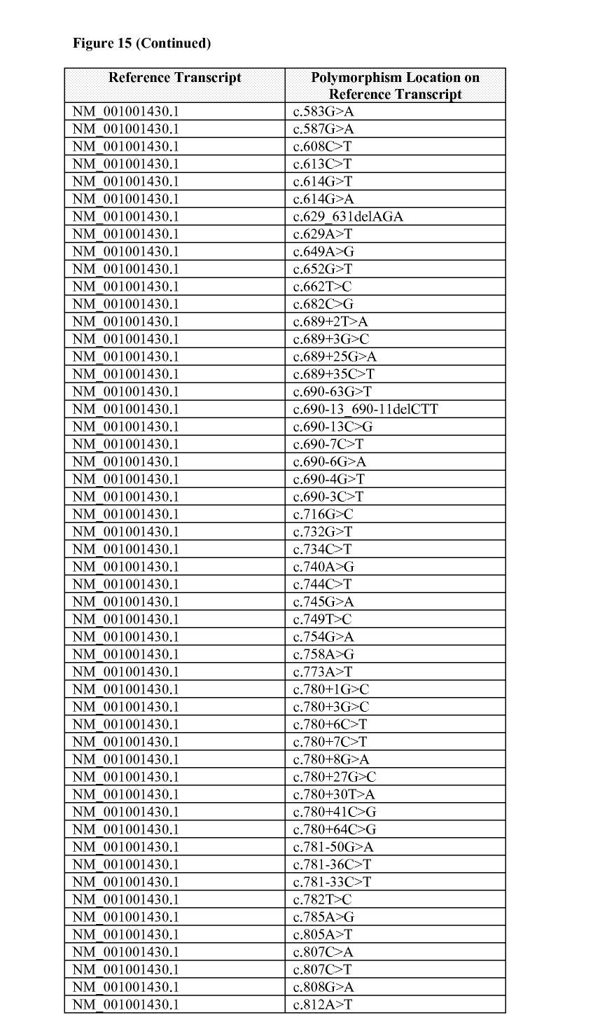

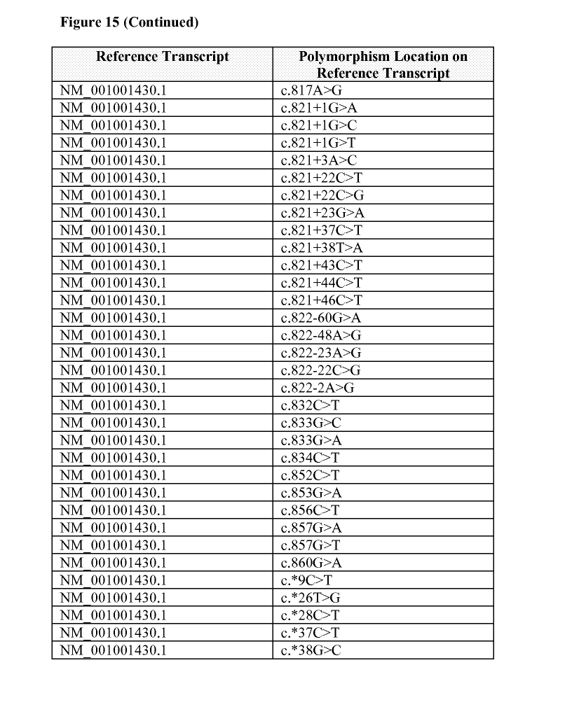

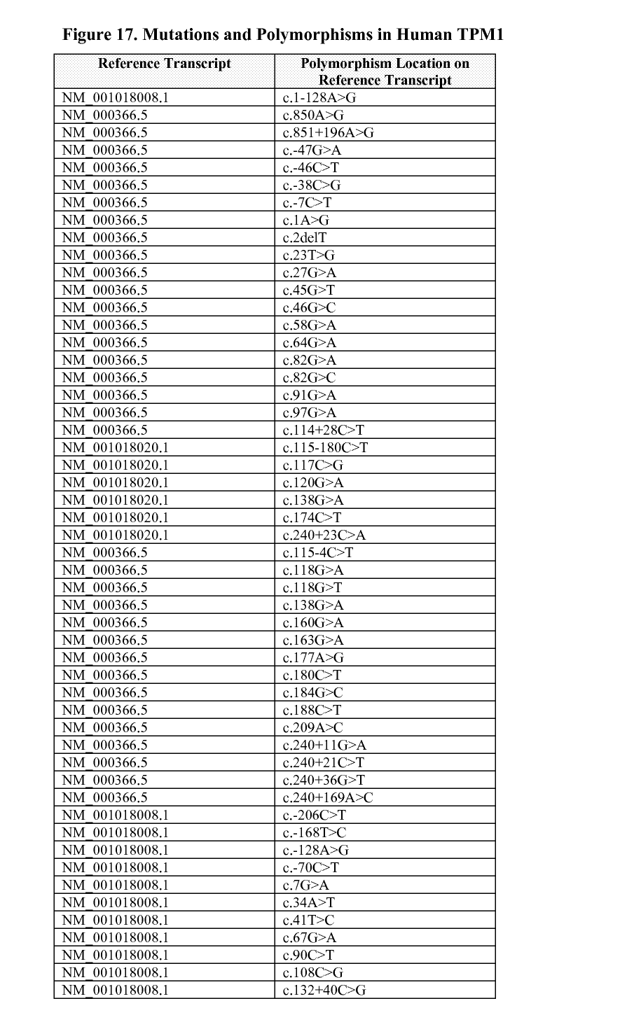

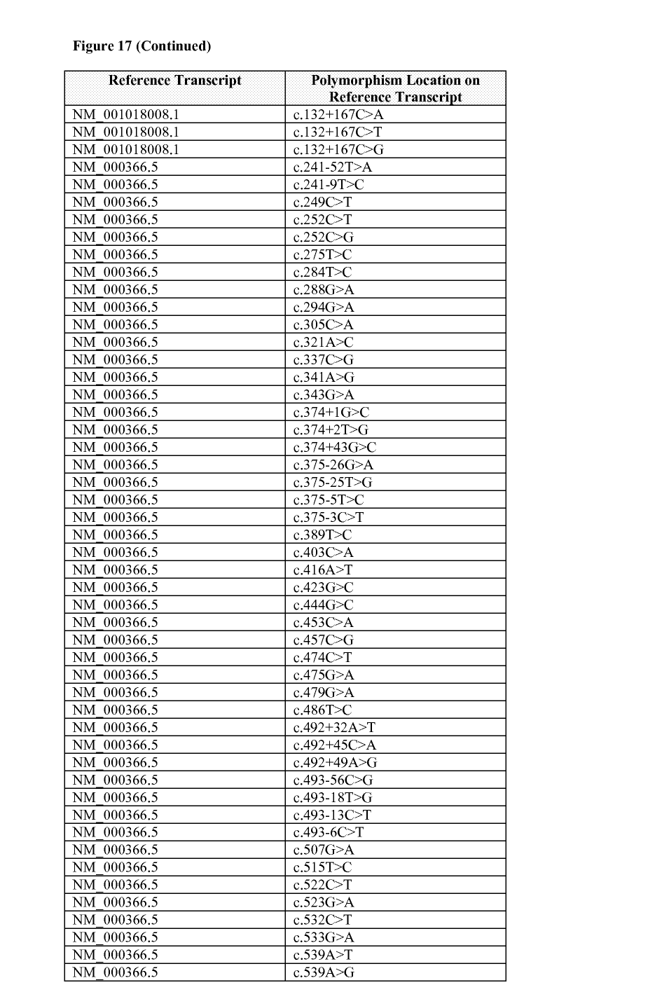

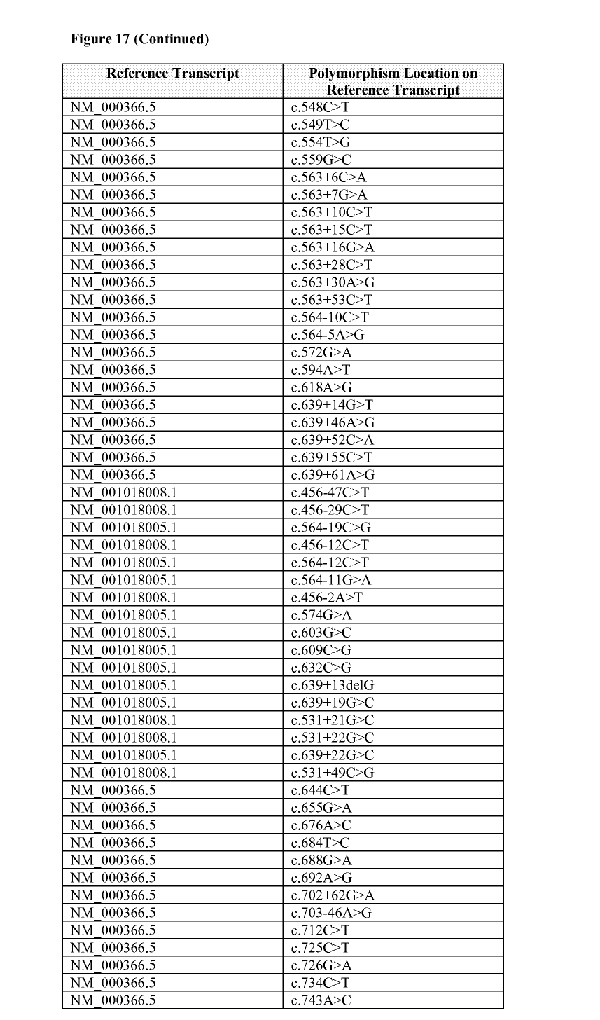

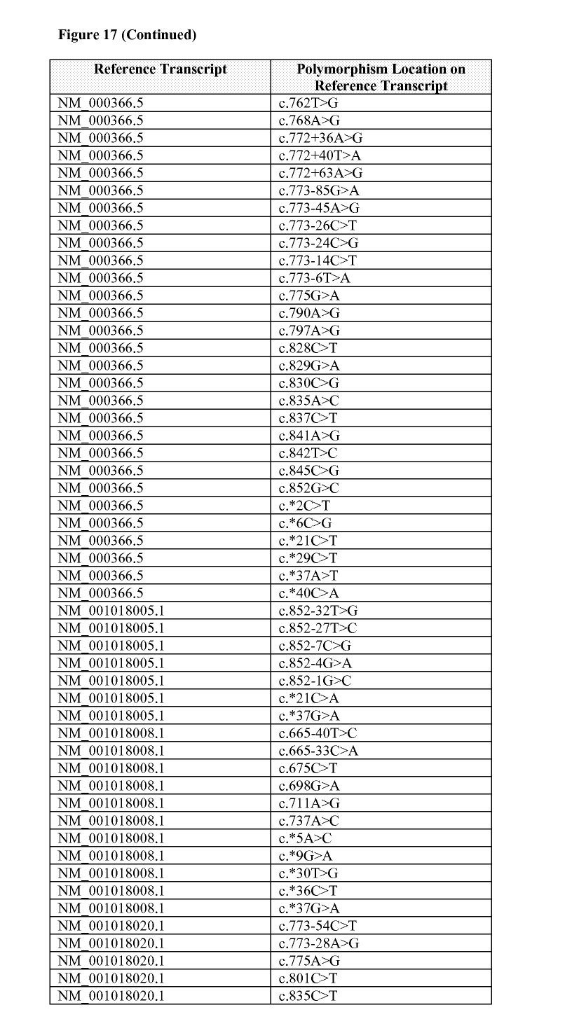

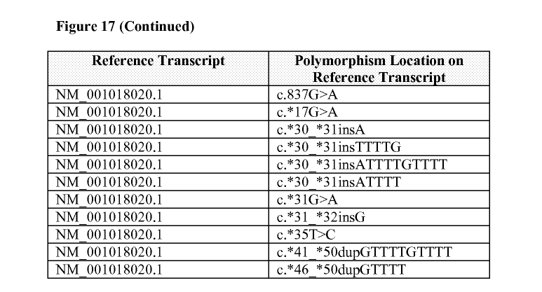

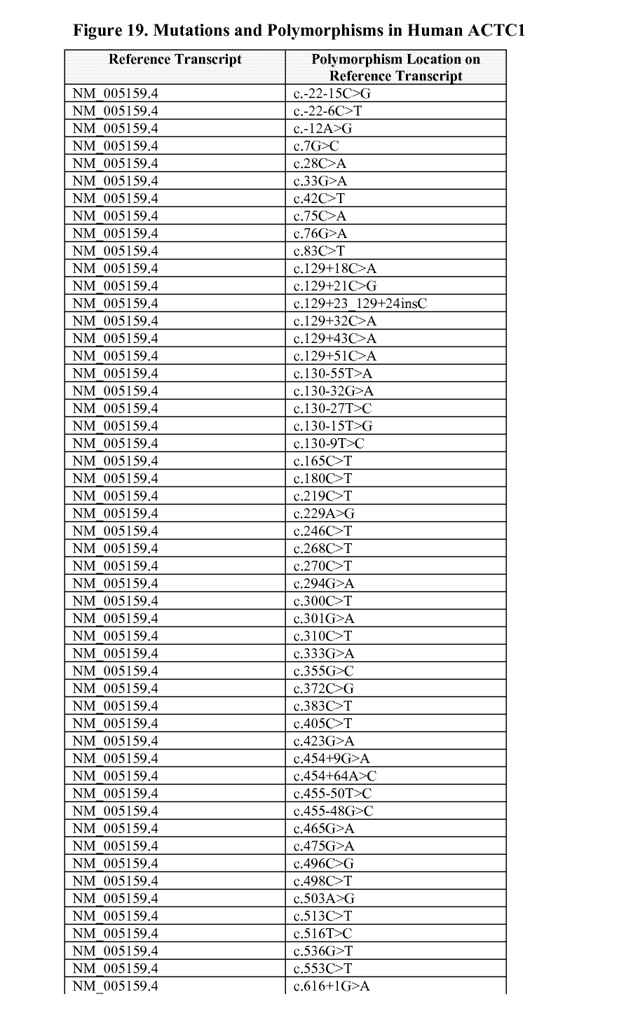

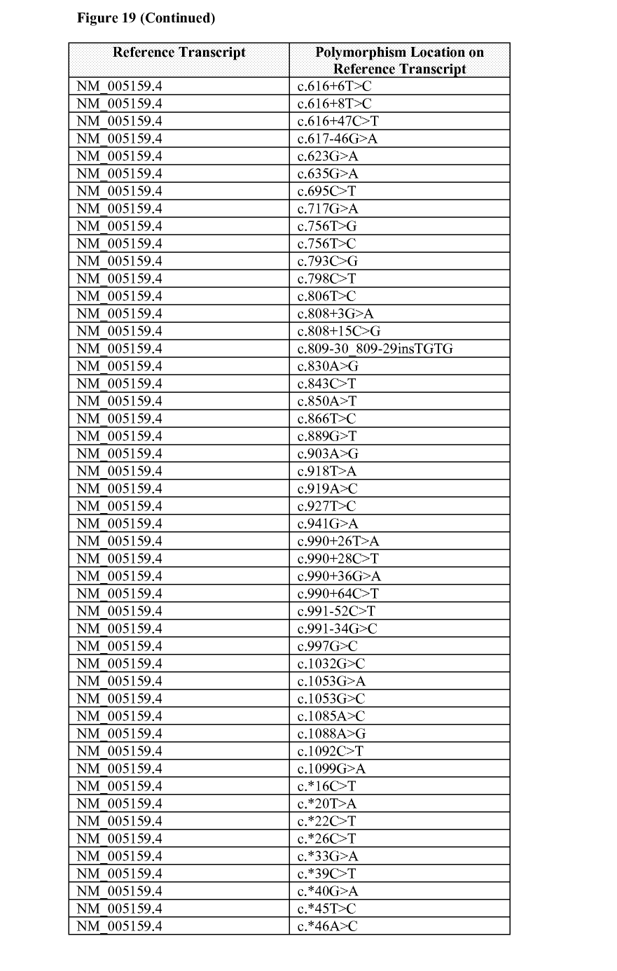

Disease causing mutations are not the only polymorphisms present in MYH7, MYBPC3, TNNT2, TNNI3, ACTC1, MYL2, MYL3 and TPM1. Known polymorphisms, e.g., single-nucleotide polymorphisms (SNPs), in these genes are provided in FIGS. 7, 9, 11, 13, 15, 17 and 19. To selectively prevent translation of a transcript carrying a disease causing mutation it is not necessary to target the actual mutation. Rather, any polymorphism present on the transcript carrying the disease-causing mutation but that is not present on the other transcript can be targeted. Often, the subject to be treated with a composition described herein will have been diagnosed as carrying a disease-causing mutation through the sequencing of their MYH7, MYBPC3, TNNT2, TNNI3, ACTC1, MYL2, MYL3 and/or TPM1 genes. The resulting sequences can be used to select inhibitory antisense molecules (e.g., RNAi molecules) that selectively target transcripts carrying disease-causing mutations. Developing antisense molecules that target allelic-specific, common polymorphisms rather than each patient's specific mutation overcomes the challenge of producing thousands of antisense molecules, e.g., RNAi, that would be required to silence each unique HCM, DCM or LVNC mutation.

### DEFINITIONS

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “ACTC1” refers to the actin, alpha, cardiac muscle 1 gene. ACTC1 is found on Chromosome 15 at position 35080297-35087927 of NC 000015.9.

As used herein, the term “administering” means providing a pharmaceutical agent or composition to a subject, and includes, but is not limited to, administering by a medical professional and self-administering.

As used herein, the terms “antisense oligomer” or “antisense compound” or “antisense molecule” or “antisense oligonucleotide” or “oligonucleotide” are used interchangeably and refer to a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Antisense molecules include, for example, peptide nucleic acids (PNAs), locked nucleic acids (LNAs), 2′-O-Methyl oligonucleotides and RNA interference agents (siRNA agents). Such an antisense oligomer can be designed to block or inhibit translation of mRNA or to inhibit natural pre-mRNA splice processing, or induce degradation of targeted mRNAs, and may be said to be “directed to” or “targeted against” a target sequence with which it hybridizes. The target sequence may be within an exon or within an intron. The target sequence for a splice site may include an mRNA sequence having its 5′ end 1 to about 25 base pairs downstream of a normal splice acceptor junction in a preprocessed mRNA. In some embodiments, the splice site target sequence is any region of a preprocessed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

As used herein, the term “dilated cardiomyopathy” or “DCM” refers to a cardiomyopathy in which the heart becomes weakened and enlarged and cannot pump blood efficiently. DCM is caused by mutations in certain genes, including MYH7, TNNT2, TNNI3, ACTC1, and TPM1. Exemplary dominant disease causing mutations are provided in FIGS. 8, 14, 16, 18 and 20.

As used herein, the term “hypertrophic cardiomyopathy” or “HCM” refers to the disease of the myocardium in which a portion of the myocardium is hypertrophied. HCM is caused by mutations in certain genes, including MYH7, MYBPC3, TNNT2, TNNI3, ACTC1, MYL2, MYL3 and TPM1. Exemplary dominant disease causing mutations are provided in FIGS. 8, 10, 12, 14, 16, 18 and 20.

As used herein, the terms “interfering RNA molecule”, “inhibiting RNA molecule” and “RNAi molecule” are used interchangeably. Interfering RNA molecules include, but are not limited to, siRNA molecules, single-stranded siRNA molecules and shRNA molecules. Interfering RNA molecules generally act by forming a heteroduplex with the target molecule, which is selectively degraded or “knocked down,” hence inactivating the target RNA. Under some conditions, an interfering RNA molecule can also inactivate a target transcript by repressing transcript translation and/or inhibiting transcription of the transcript.

As used herein, the terms “inactivating a target RNA” or “inactivating a target transcript” refer to a decrease in the RNA or transcript levels associated with the formation of a heteroduplex between the interfering RNA and the target RNA or target transcript.

As used herein, the term “left ventricular non compaction” or “LVNC” refers to a non-compaction cardiomyopathy (spongiform cardiomyopathy) in which the ventricles, and particularly the left ventricle, fails to undergo full compaction. LVNC is caused by mutations in certain genes, including MYH7. Exemplary dominant disease causing mutations are provided in FIG. 8.

As used herein, the term “MYH7” refers to the myosin, heavy chain 7, cardiac muscle, beta gene. MYH7 is found on Chromosome 14 at position 23881947-23904870 of NC_000014.8.

As used herein, the term “MYL2” refers to the myosin, light chain 2, regulatory, cardiac, slow gene. MYL2 is found on Chromosome 12 at position 111348623-111358404 of NC_000012.11.

As used herein, the term “MYL3” refers to the myosin, light chain 3, alkali, ventricular, skeletal, slow gene. MYL3 is found on Chromosome 3 at position 46899357-46904973 of NC_000003.11.

The term “operably linked” as used herein means placing an oligomer-encoding sequence under the regulatory control of a promoter, which then controls the transcription of the oligomer.

A “patient” or “subject” refers to either a human or a non-human animal. As used herein, the term “polymorphism” refers to an allele-specific variation in the nucleic acid sequence of a gene. Exemplary polymorphisms in HCM, DCM and LVNC associated genes are provided in FIGS. 7, 9, 11, 13, 15, 17 and 19. As used herein, the term “mutation” refers to a polymorphism associated with a disease. Exemplary mutations associated with HCM, DCM and LVNC are provided in FIGS. 8, 10, 12, 14, 16, 18 and 20.

The terms “polynucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

As used herein, the term “poison-polypeptide” refers to a mutant protein that differs from the normal protein in such a way that that it becomes incorporated into the sarcomere or contractile unit and has aberrant function thereby leading to disease.

The term “reduce” or “inhibit” when used in reference to a disease or condition relates generally to the ability of one or more antisense (e.g., RNAi) compounds to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of, e.g., HCM, LVNC, DCM. A “decrease” in a response may be “statistically significant” as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

An oligonucleotide “specifically hybridizes” to a target polynucleotide if the oligomer hybridizes to the target under physiological conditions, with a Tm substantially greater than 45° C., or at least 50° C., or at least 60° C., or at least 80° C. or higher. Such hybridization corresponds to stringent hybridization conditions. At a given ionic strength and pH, the Tm is the temperature at which 50% of a target sequence hybridizes to a complementary polynucleotide. Again, such hybridization may occur with “near” or “substantial” complementarity of the antisense oligomer to the target sequence, as well as with exact complementarity.

The term “target sequence” refers to a portion of the target RNA against which the oligonucleotide or antisense agent is directed, that is, the sequence to which the oligonucleotide will hybridize by Watson-Crick base pairing of a complementary or mostly-complementary sequence. In certain embodiments, the target sequence may comprise a disease associated mutation or polymorphism of MYH7, MYL2, MYL3, TNNI3, TNNT2, TPM1, or ACTC1.

The term “targeting sequence” or “antisense targeting sequence” refers to the sequence in an oligonucleotide or other antisense agent that is complementary (meaning, in addition, substantially complementary) to the target sequence in the RNA genome. The entire sequence, or only a portion, of the antisense compound may be complementary to the target sequence. For example, in an oligonucleotide having 20-30 bases, about 6, 7, 8, 9, 10, 11, 12, 13, 14, 15, 16, 17, 18, 19, 20, 21, 22, 23, 24, 25, 26, 27, 28, or 29 may be targeting sequences that are complementary to the target region. Typically, the targeting sequence is formed of contiguous bases, but may alternatively be formed of non-contiguous sequences that when placed together, e.g., from opposite ends of the oligonucleotide, constitute sequence that spans the target sequence.

The phrases “therapeutically-effective amount” and “effective amount” as used herein means that amount of a compound, material, or composition comprising a compound described herein which is effective for producing some desired therapeutic effect in at least a sub-population of cells in an animal at a reasonable benefit/risk ratio applicable to any medical treatment.

As used herein, the term “TNNI3” refers to the troponin I type 3 (cardiac) gene. TNNI3 is found on Chromosome 19 at position 55663135-55669100 of NC_000019.9.

As used herein, the term “TNNT2” refers to the troponin T type 2 (cardiac) gene. TNNT2 is found on Chromosome 1 at position 201328136-201346836 of NC_00001.10.

As used herein, the term “TPM1” refers to the tropomyosin 1 (alpha) gene. TPM1 is found on Chromosome 15 at position 63334838-63364114 of NC_000015.9.

“Treating” a disease in a subject or “treating” a subject having a disease refers to subjecting the subject to a pharmaceutical treatment, e.g., the administration of a composition disclosed or contemplated herein, such that at least one symptom of the disease is decreased or prevented from worsening.

### Antisense Oligonucleotide Compositions

In some embodiments, antisense oligonucleotide compounds are provided herein. In particular embodiments, antisense oligonucleotide compounds contain a base sequence targeting a MYH7, MYBPC3, MYL2, MYL3, TNNI3, TNNT2, TPM1, and/or ACTC1. In some embodiments, the antisense oligonucleotide compounds contain a base sequence targeting a MYH7, MYBPC3, MYL2, MYL3, TNNI3, TNNT2, TPM1, and/or ACTC1 comprising a mutation or polymorphism described or contemplated herein. In certain embodiments, antisense targeting sequences are designed to hybridize to a region of one or more of the target sequences listed in FIGS. 7-20. Selected antisense targeting sequences can be made shorter, e.g., about 12 bases, or longer, e.g., about 40 bases, and include a small number of mismatches, as long as the sequence is sufficiently complementary to effect splice modulation upon hybridization to the target sequence, and optionally forms with the RNA a heteroduplex having a Tm of 45° C. or greater.

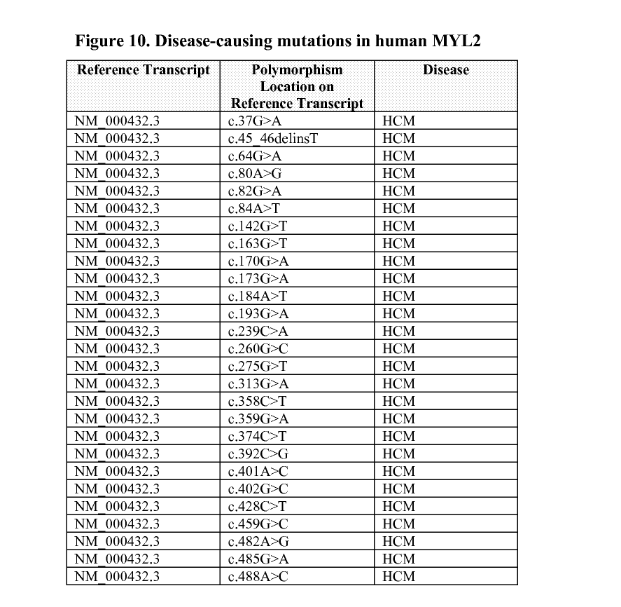

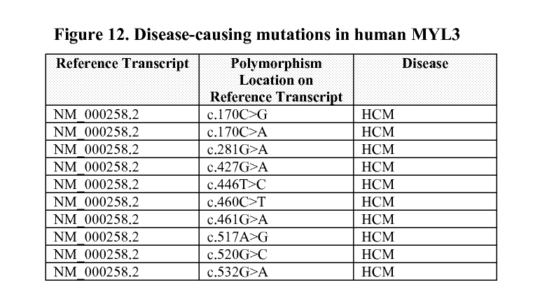

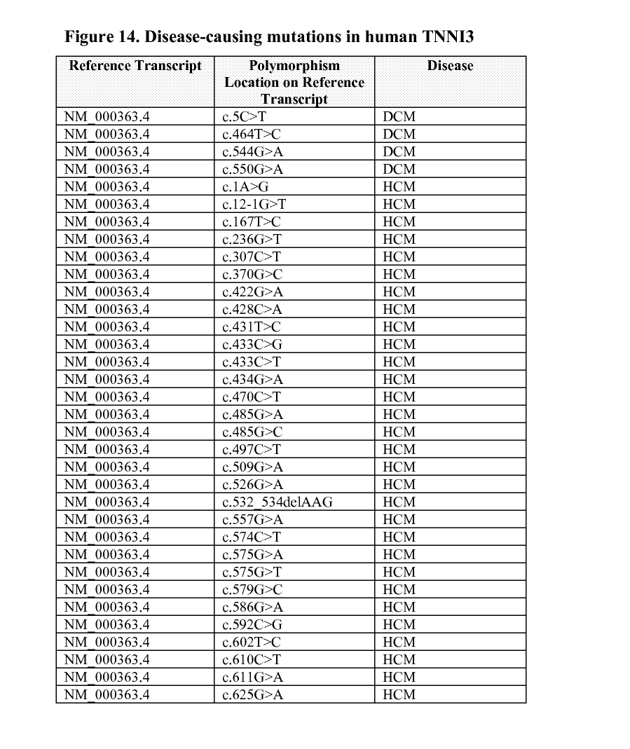

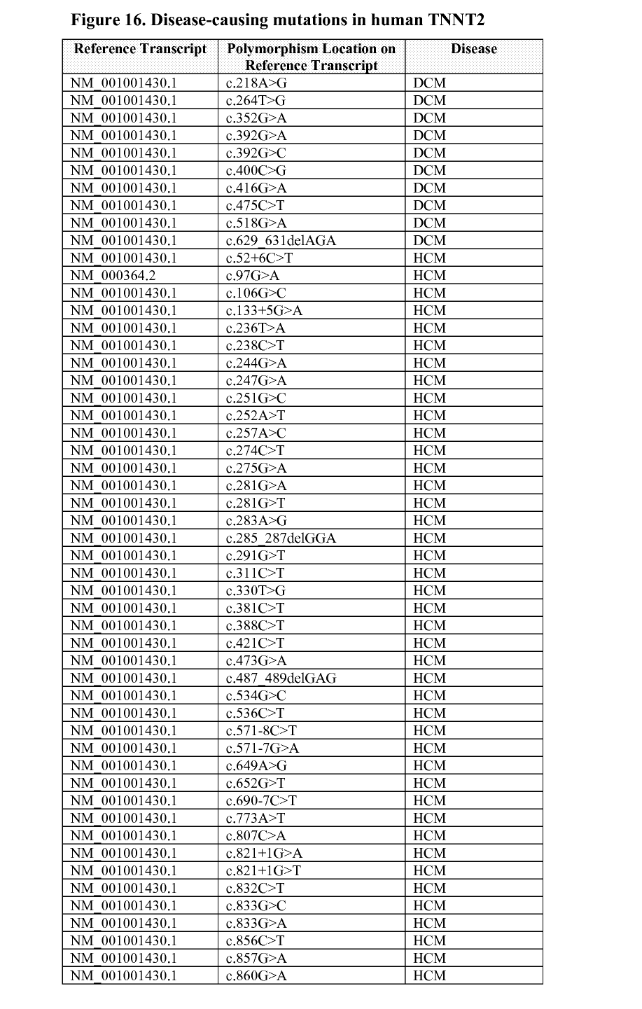

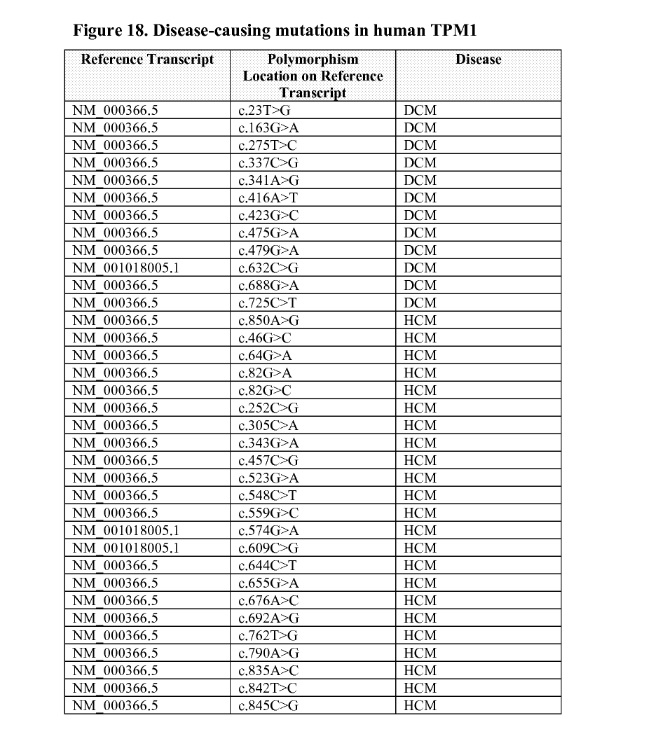

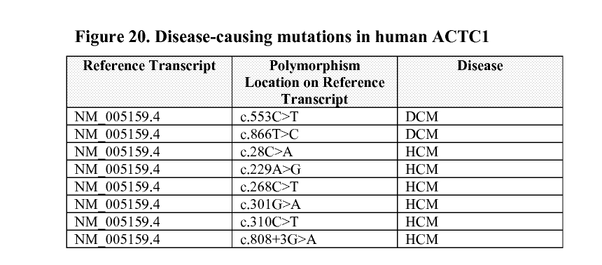

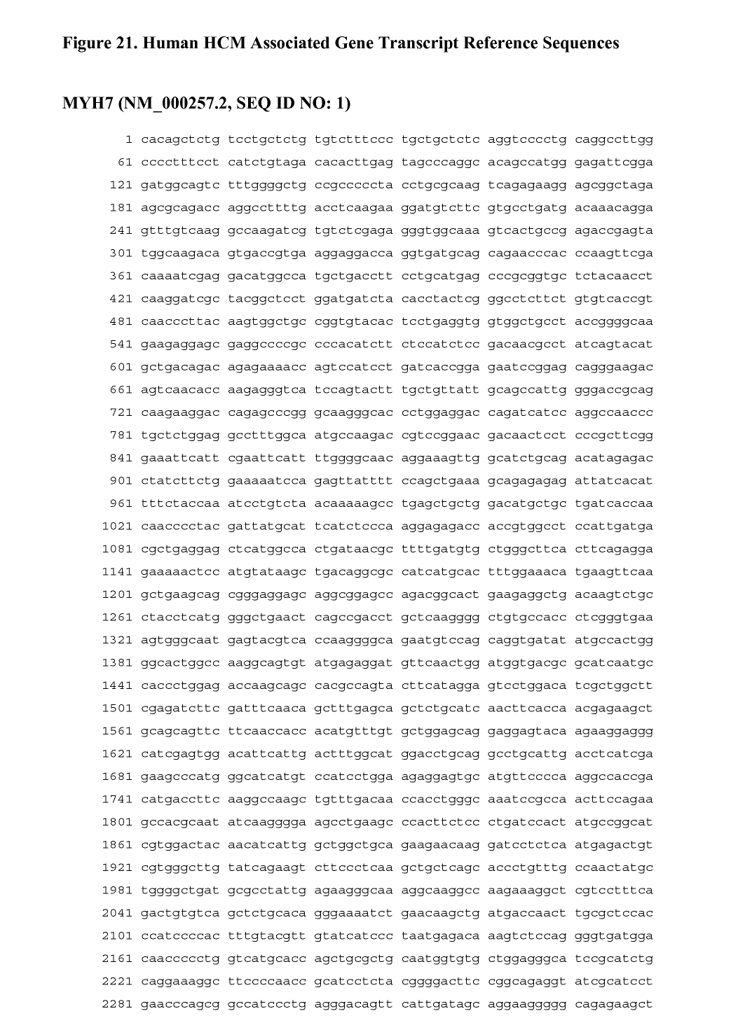

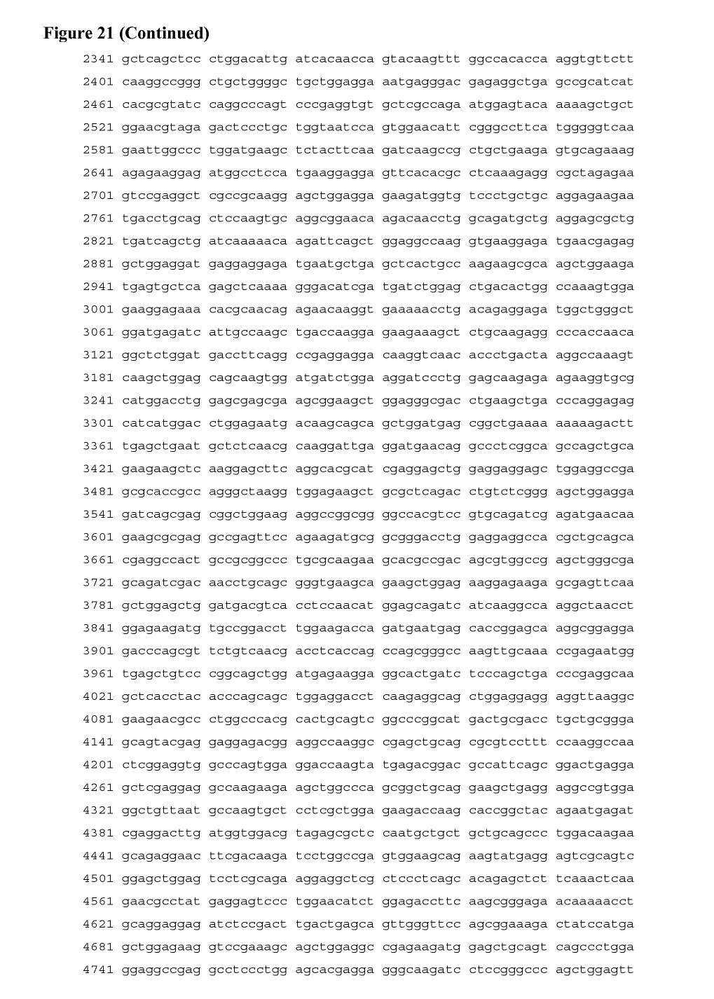

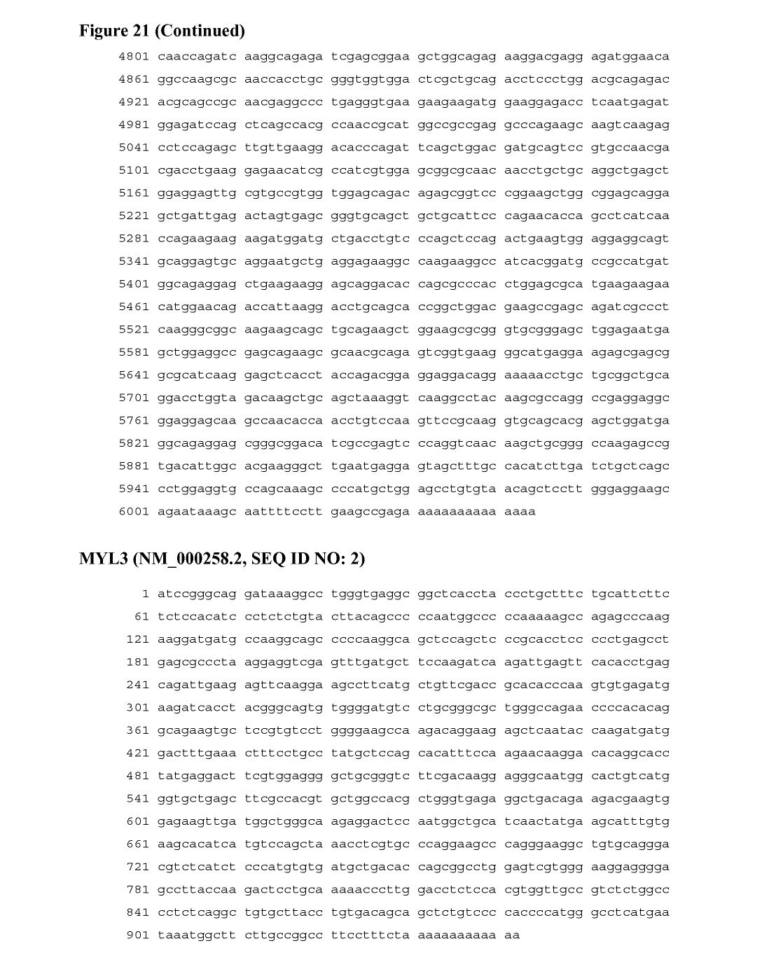

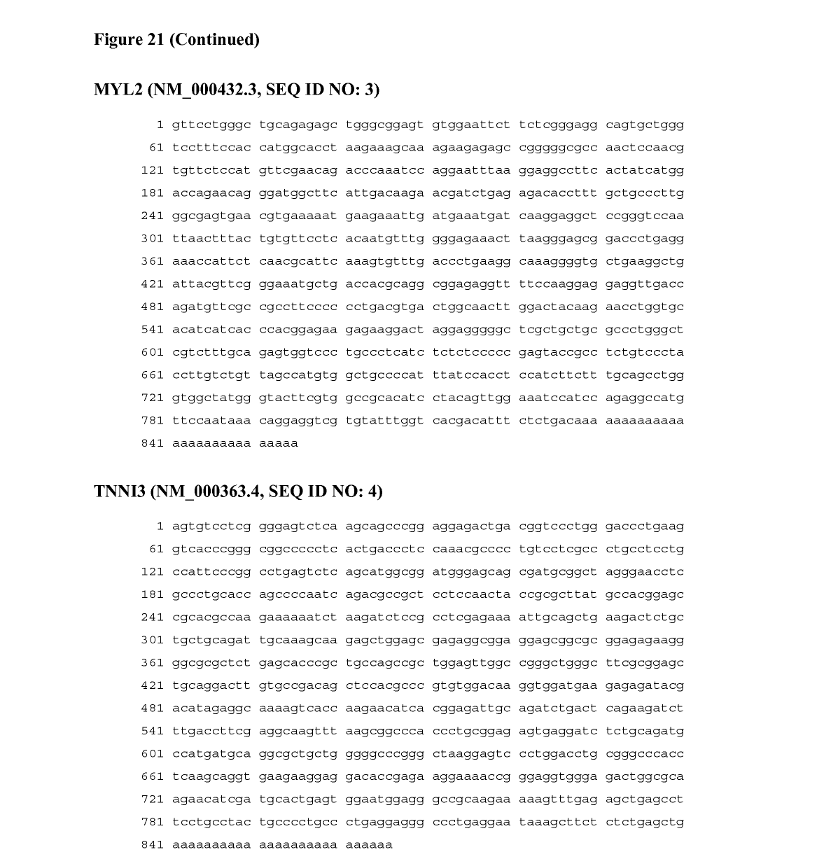

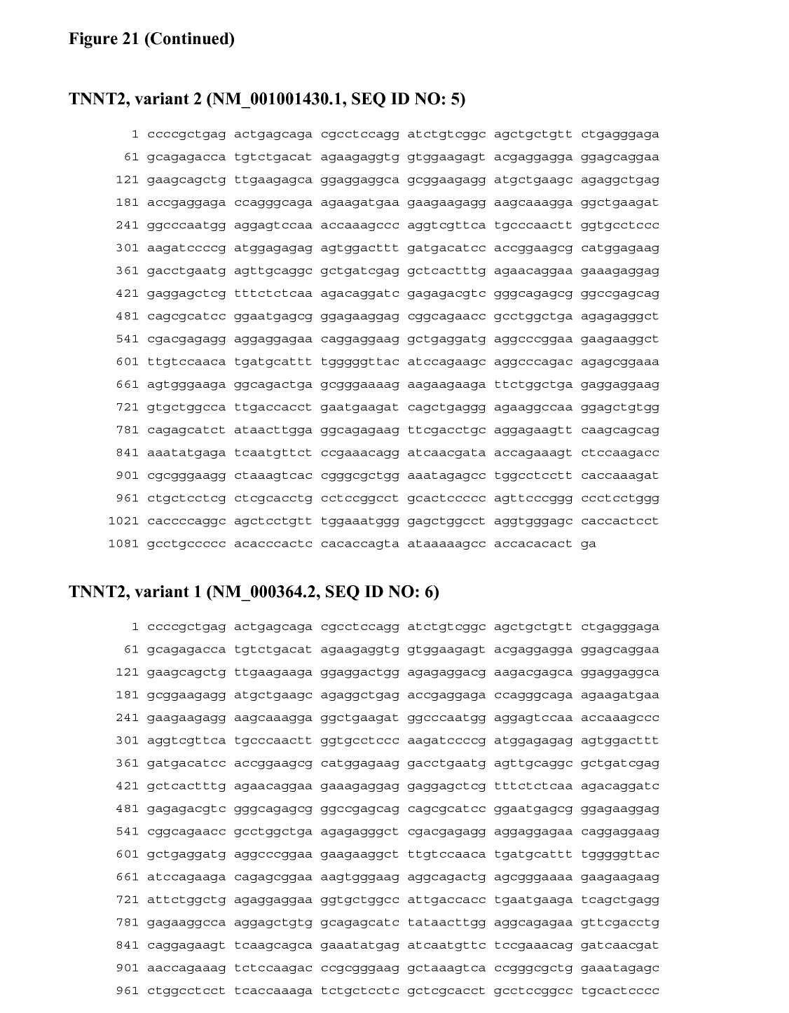

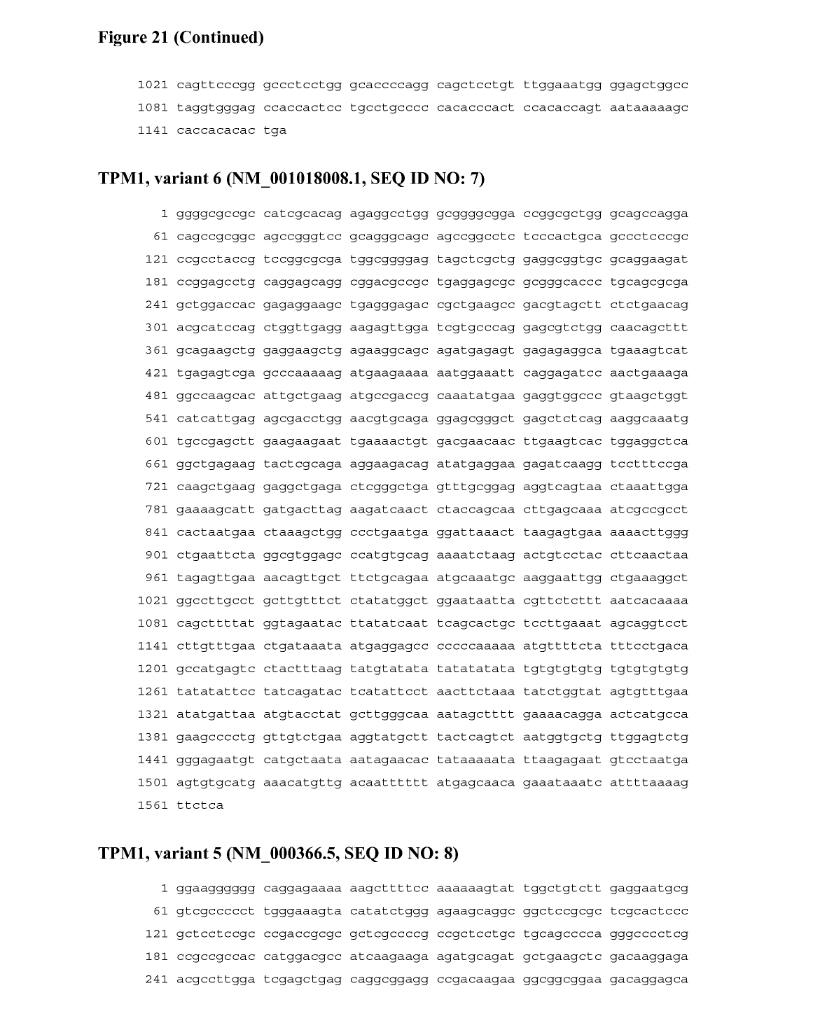

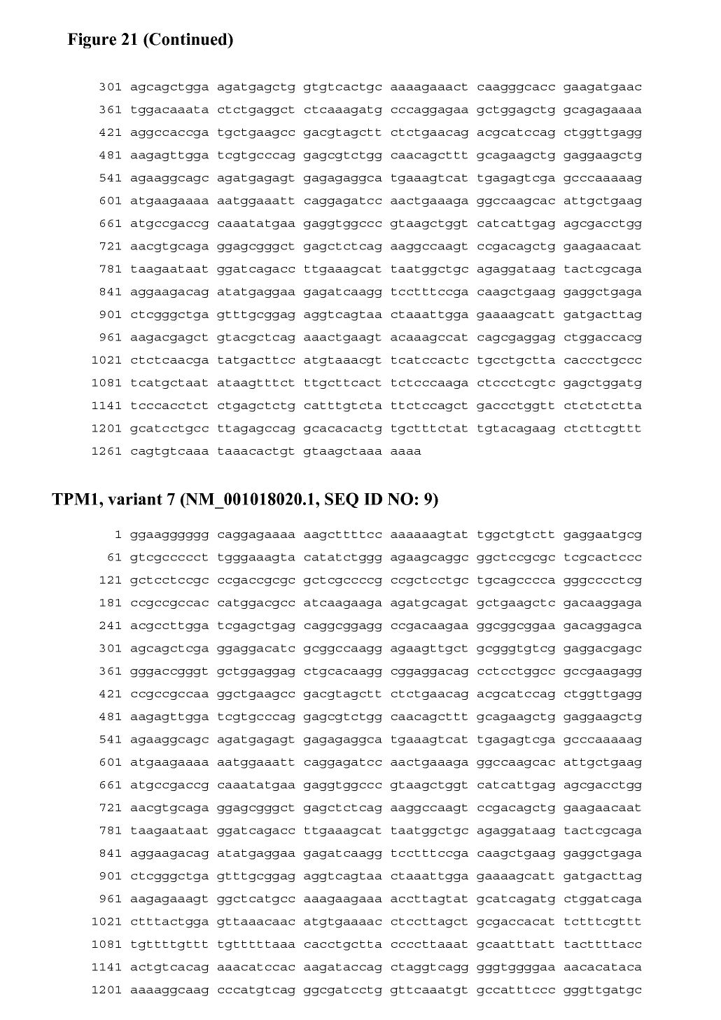

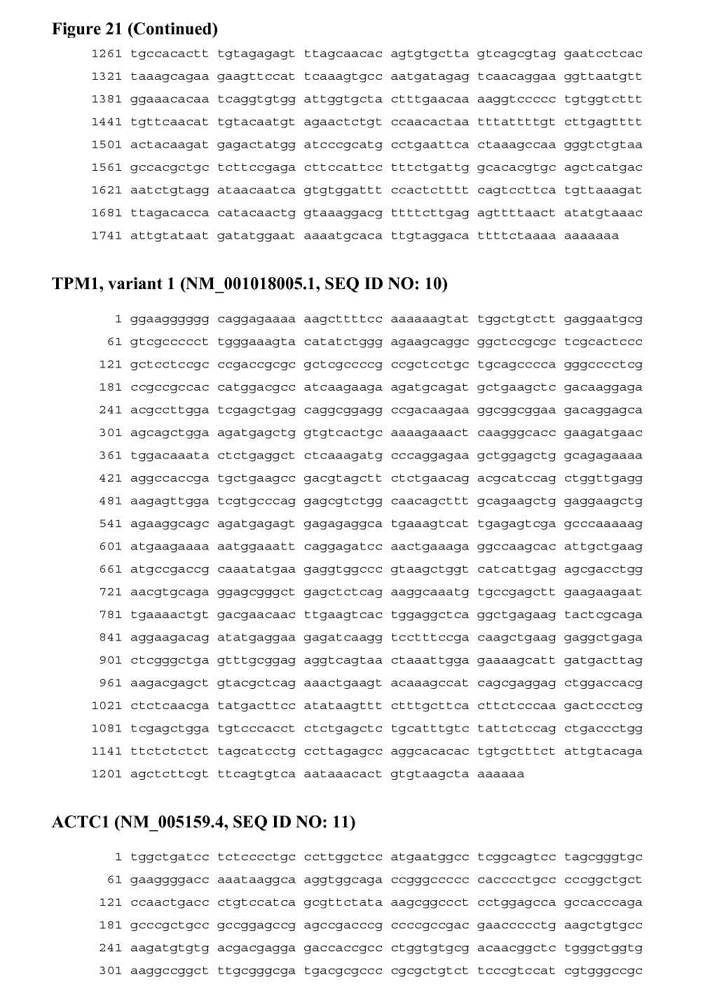

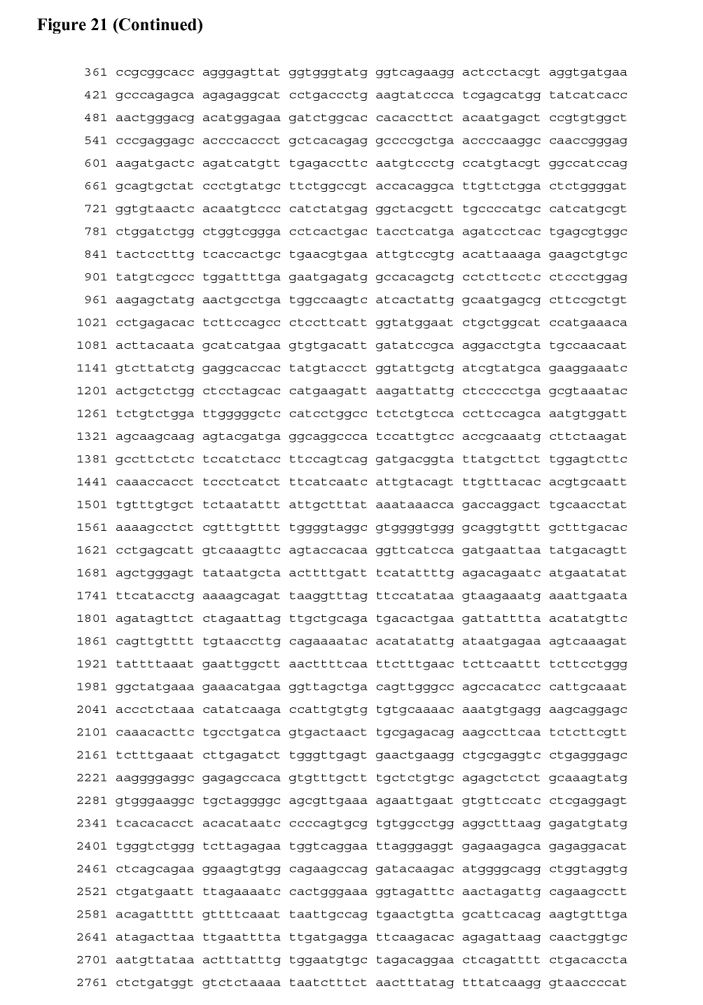

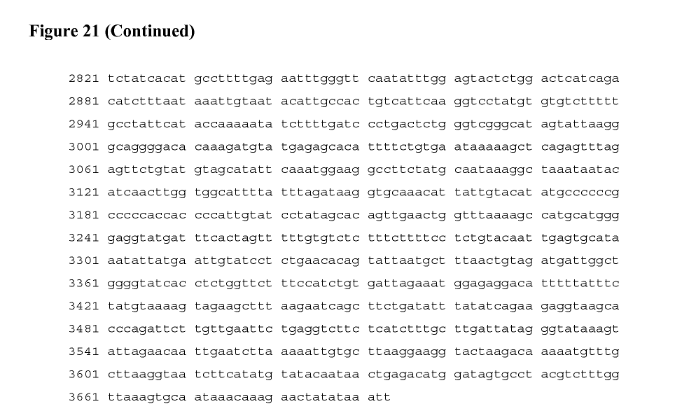

The target sequence may comprise, bridge, or overlap a mutation or polymorphism (e.g., a single-nucleotide polymorphism (SNP)) present in MYH7, MYBPC3, MYL2, MYL3, TNNI3, TNNT2, TPM1, or ACTC1. In one embodiment, the target sequence is a disease associated mutation in MYH7, MYBPC3, MYL2, MYL3, TNNI3, TNNT2, TPM1, or ACTC1. In one embodiment, the disease associated mutation present in MYH7 is provided in FIG. 8. In another embodiment, the disease associated mutation present in MYL2 is provided in FIG. 10. In one embodiment, the disease associated mutation present in MYL3 is provided in FIG. 12. In another embodiment, the disease associated mutation present in TNNI3 is provided in FIG. 14. In another embodiment, the disease associated mutation present in TNNT2 is provided in FIG. 16. In one embodiment, the disease associated mutation present in TPM1 is provided in FIG. 18. In another embodiment, the disease associated mutation present in ACTC1 is provided in FIG. 20.

Polymorphisms such as SNPs include mutations in coding and non-coding regions, as well as intergenic sequences. Furthermore, the polymorphisms include nucleotide substitutions, deletions, and/or additions, including those that result in missense and nonsense mutations. By targeting allele-specific, common polymorphisms rather than each specific disease causing mutation on a patient-by-patient basis, a single antisense molecule (e.g., siRNA or shRNA) can be used to inhibit expression of more than one disease associated mutation in more than one patient. Accordingly, in one embodiment, the target sequence comprises a polymorphism, e.g., a SNP, in an allele of MYH7, MYBPC3, MYL2, MYL3, TNNI3, TNNT2, TPM1, or ACTC1 comprising a disease associated mutation. In one embodiment, the target sequence comprises the polymorphism, but not the disease associated mutation. In some embodiments, the polymorphism is present in the mutated allele of MYH7, MYBPC3, MYL2, MYL3, TNNI3, TNNT2, TPM1, or ACTC1, but not in a corresponding allele of MYH7, MYBPC3, MYL2, MYL3, TNNI3, TNNT2, TPM1, or ACTC1 that does not comprise the disease associated mutation.

Accordingly, in one embodiment, the antisense oligonucleotide selectively inhibits an allele comprising a disease associated mutation in comparison to an allele that does not comprise the disease associated mutation (e.g., a wild-type allele). By targeting a polymorphism, e.g., a SNP, that differentiates mutant and wild-type alleles certain antisense oligonucleotides could be used to silence different, patient-specific mutations in the same gene. In another embodiment, an antisense oligonucleotide that targets a polymorphism that distinguishes mutant and wild-type alleles is used in combination with an antisense oligonucleotide that targets a disease associated mutation.

In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an antisense oligonucleotide is not necessarily 100% complementary to the target sequence, it is effective to stably and specifically bind to the target sequence, such that splicing of the target pre-RNA is modulated.

The antisense oligonucleotides can employ a variety of antisense chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Antisense oligonucleotide compounds may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel et al., Chemical Communications (1998) 455; Wengel et al., Tetrahedron (1998) 54:3607, and Wengel et al., Accounts of Chem. Research (1999) 32:301); Obika, et al., Tetrahedron Letters (1997) 38:8735; (1998) 39:5401, and Obika, et al., Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds comprise alternating LNA and DNA subunits where the intersubunit linker is phosphorothioate, for example.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases Si and P 1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

### Interfering RNA Molecules

In certain embodiments, interfering RNA molecules that selectively target MYH7, MYBPC3, MYL2, MYL3, TNNT2, TNNI3, ACTC1, and/or TPM1 transcripts carrying particular polymorphisms or disease associated mutations (e.g., antisense molecules, siRNA, single-stranded siRNA molecules, shRNA molecules, ribozymes or triplex molecules) are provided herein and/or used in methods described herein.

In certain embodiments, the RNAi oligonucleotide is single stranded. In other embodiments RNAi oligonucleotide, is double stranded. Certain embodiments may employ short-interfering RNAs (siRNA). In certain embodiments, the first strand of the double-stranded oligonucleotide contains two more nucleoside residues than the second strand. In other embodiments, the first strand and the second strand have the same number of nucleosides; however, the first and second strands are offset such that the two terminal nucleosides on the first and second strands are not paired with a residue on the complimentary strand. In certain instances, the two nucleosides that are not paired are thymidine resides.

Interfering RNA molecules provided herein can contain non-RNA bases. For example, interfering RNA molecules provided herein can contain DNA bases or non-naturally occurring nucleotides. Such molecules are useful, for example, in methods of treating HCM, DCM and/or LVNC.

In certain embodiments, the interfering RNA molecules selectively inhibit expression a gene transcript carrying a mutation compared to a gene transcript not carrying a mutation (e.g., a mutation listed in FIG. 8, 10, 12, 14, 16, 18 or 20). In some embodiments, an interfering RNA molecule inhibits or decreases expression of the transcript comprising the mutation at least 1.25 times, 1.5 times, 1.75 times, 2 times, 2.25 times, 2.5 times, 2.75 times or 3 times as much as it inhibits or decreases the expression of the transcript not comprising the mutation. In other embodiments, the interfering RNA molecule inactivates the transcript comprising the mutation at least 1.25 times, 1.5 times, 1.75 times, 2 times, 2.25 times, 2.5 times, 2.75 times or 3 times as much as it inactivates the transcript not comprising the mutation. In some embodiments, the interfering RNA molecule inhibits or decreases expression of the transcript comprising the mutation by at least 20%, 25%, 30%, 35%, 40%, 45%, 50%, 55%, 60%, 65%, 70%, 75%, 80%, 85%, 90% or 95%. In some embodiments, the interfering RNA molecule inactivates the transcript not comprising the mutation by at no more than 5%, 10%, 15%, 20%, 25%, 30%, 35%, 40%, 45% or 50%.

In some embodiments, the interfering RNA molecule binds to the transcript carrying the mutation at the position of the mutation. In some embodiments, the interfering RNA molecule binds to the transcript carrying the mutation at the position of a polymorphism that is distinct from the mutation.

In some embodiments, the interfering RNA molecule comprises a nucleotide sequence that is complementary to a nucleotide sequence of the transcript except for a single nucleotide mismatch at a position outside of a polymorphism or mutation. In some embodiments, the interfering RNA molecule comprises a nucleic acid sequence of 21 nucleotides in length, wherein 20 nucleotides of the nucleic acid sequence are complementary to the transcript comprising the mutation and no more than 19 nucleotides of the nucleic acid sequence are complementary to the transcript not comprising the mutation.

The interfering RNA described herein may be contacted with a cell or administered to an organism (e.g., a human). Alternatively, constructs and/or vectors encoding the interfering RNA molecules may be contacted with or introduced into a cell or organism. In certain embodiments, a viral, retroviral or lentiviral vector is used. In some embodiments, the vector has a tropism for cardiac tissue. In some embodiments, the vector is an adeno-associated virus (AAV).

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering RNA contains a 1, 2 or 3 nucleotide mismatch with the target sequence. The RNA interference molecule may have a 2 nucleotide 3′ overhang. If the RNA interference molecule is expressed in a cell from a construct, for example from a hairpin molecule or from an inverted repeat of the desired sequence, then the endogenous cellular machinery will create the overhangs. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

siRNA

In instances when the interfering RNA molecule comprises siRNA, the molecule should include a region of sufficient homology to the target region, and be of sufficient length in terms of nucleotides, such that the siRNA molecule, or a fragment thereof, can down-regulate target RNA. The term “ribonucleotide” or “nucleotide” can, in the case of a modified RNA or nucleotide surrogate, also refer to a modified nucleotide, or surrogate replacement moiety at one or more positions. Thus, an siRNA molecule is or includes a region that is at least partially complementary to the target RNA. It is not necessary that there be perfect complementarity between the siRNA molecule and the target, but the correspondence must be sufficient to enable the siRNA molecule to direct sequence-specific silencing, such as by RNAi cleavage of the target RNA. Some embodiments include one or more with respect to the target RNA. In some embodiments, the mismatches may be in a terminal region or regions, e.g., within 6, 5, 4, or 3 nucleotides of the 5′ and/or 3′ terminus of the siRNA molecule. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Interfering RNA molecules may include, for example, molecules that are long enough to trigger the interferon response (which can be cleaved by Dicer (Bernstein et al., Nature, 409:363-366, 2001) and enter a RISC (RNAi-induced silencing complex)), in addition to molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA molecules herein. In some embodiments, siRNA molecules have a duplexed region of less than 60, 50, 40, or 30 nucleotide pairs.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 20, 21, 22, 23, 24, or 25 nucleotide pairs, and one or more overhangs, such as one or two 3′ overhangs, of 2-3 nucleotides.

In addition to homology to target RNA and the ability to down regulate a target gene, an siRNA molecule may have one or more of the following properties: it may, despite modifications, even to a very large number, or all of the nucleosides, have an antisense strand that can present bases (or modified bases) in the proper three dimensional framework so as to be able to form correct base pairing and form a duplex structure with a homologous target RNA which is sufficient to allow down regulation of the target, e.g., by cleavage of the target RNA; it may, despite modifications, even to a very large number, or all of the nucleosides, still have “RNA-like” properties, i.e., it may possess the overall structural, chemical and physical properties of an RNA molecule, even though not exclusively, or even partly, of ribonucleotide-based content. For example, an siRNA molecule can contain, e.g., a sense and/or an antisense strand in which all of the nucleotide sugars contain e.g., 2′ fluoro in place of 2′ hydroxyl. This deoxyribonucleotide-containing agent can still be expected to exhibit RNA-like properties. While not wishing to be bound by theory, the electronegative fluorine prefers an axial orientation when attached to the C2′ position of ribose. This spatial preference of fluorine can, in turn, force the sugars to adopt a C3′-endo pucker. This is the same puckering mode as observed in RNA molecules and gives rise to the RNA-characteristic A-family-type helix. Further, since fluorine is a good hydrogen bond acceptor, it can participate in the same hydrogen bonding interactions with water molecules that are known to stabilize RNA structures. In some embodiments, a modified moiety at the 2′ sugar position will be able to enter into H-bonding which is more characteristic of the OH moiety of a ribonucleotide than the H moiety of a deoxyribonucleotide.

shRNA

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC).

In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Suitable shRNA sequences can be identified, synthesized, and modified using any means known in the art for designing, synthesizing, and modifying siRNA sequences. In certain embodiments, shRNAs may silence MYH7, MYBPC3, MYL2, MYL3, TNNI3, TNNT2, TPM1, or ACTC1 gene expression.

Additional embodiments related to the shRNAs, as well as methods of designing and synthesizing such shRNAs, are described in U.S. patent application publication number 2011/0071208, the disclosure of which is herein incorporated by reference in its entirety for all purposes.

miRNA

In some embodiments, provided herein are micro RNAs (miRNAs). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

Generating Antisense and RNAi Molecules

Interfering RNA molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

In some embodiments, the methods comprise an interfering RNA molecule or an interfering RNA encoding polynucleotide can be administered to the subject, for example, as naked RNA, in combination with a delivery reagent, and/or as a nucleic acid comprising sequences that express the siRNA or shRNA molecules. In some embodiments, the nucleic acid comprising sequences that express the siRNA or shRNA molecules are delivered within vectors, e.g. plasmid, viral and bacterial vectors. Any nucleic acid delivery method known in the art can be used in the methods described herein. Suitable delivery reagents include, but are not limited to, e.g., the Minis Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering RNA delivery systems are provided in U.S. Pat. Nos. 8,283,461, 8,313,772, 8,501,930. 8,426,554, 8,268,798 and 8,324,366, each of which is hereby incorporated by reference in its entirety.

In some embodiments, of the methods described herein, liposomes are used to deliver an inhibitory oligonucleotide to a subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

In some embodiments, opsonization inhibiting moieties suitable for modifying liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonization-inhibiting moiety is a PEG, PPG, or derivatives thereof. Liposomes modified with PEG or PEG-derivatives are sometimes called “PEGylated liposomes.”

### Nucleic Acids

Nucleic acids encoding the interfering RNA molecules described herein are also provided herein. Such a nucleic acid may further be linked to a promoter and/or other regulatory sequences, as further described herein. Nucleic acids may also hybridize specifically, e.g., under stringent hybridization conditions, to a nucleic acid described herein.

Nucleic acids (e.g., encoding an siRNA, shRNA or antisense RNA, described herein in greater detail above) can be delivered to cells in culture, in vitro, ex vivo, and in vivo. The delivery of nucleic acids can be by any technique known in the art including viral mediated gene transfer, liposome mediated gene transfer, direct injection into a target tissue, organ, or tumor, injection into vasculature which supplies a target tissue or organ.

Polynucleotides can be administered in any suitable formulations known in the art. These can be as virus particles, as naked DNA, in liposomes, in complexes with polymeric carriers, etc. Polynucleotides can be administered to the arteries which feed a tissue or tumor.

Nucleic acids can be delivered in any desired vector. A polynucleotide can be contained in a vector, which can facilitate manipulation of the polynucleotide, including introduction of the polynucleotide into a target cell. The vector can be a cloning vector, which is useful for maintaining the polynucleotide, or can be an expression vector, which contains, in addition to the polynucleotide, regulatory elements useful for expressing the polynucleotide and, where the polynucleotide encodes an RNA, for expressing the encoded RNA in a particular cell, either for subsequent translation of the RNA into a polypeptide or for subsequent trans regulatory activity by the RNA in the cell. An expression vector can contain the expression elements necessary to achieve, for example, sustained transcription of the encoding polynucleotide, or the regulatory elements can be operatively linked to the polynucleotide prior to its being cloned into the vector.

An expression vector generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, alpha virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb 25:37-42, 1993; Kirshenbaum et al., J. Clin. Invest 92:381-387, 1993; each of which is incorporated herein by reference). Exemplary types of viruses include HSV (herpes simplex virus), AAV (adeno-associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

In one embodiment, the nucleic acid encoding the antisense or RNAi molecule described herein is delivered in a viral vector. In some embodiments, the viral vector is an AAV vector. Examples of AAV vectors include, but are not limited to, AAV1, AAV2, AAV3, AAV4, AAV5, AAV6, AAV7, AAV8, AAV9, AAV10, and AAV11 or variants thereof. In one embodiment, the viral vector is AAV9 or a variant thereof.

In one embodiment, the expression of the nucleic acid encoding the antisense molecule (e.g., siRNA or shRNA) is driven by a tissue-specific promoter. In some embodiments, the tissue-specific promoter is a cardiac-specific promoter. Examples of cardiac-specific promoters include, but are not limited to, cardiac troponin T promoter (cTnT), cardiac α-actin promoter, myosin light chain-2v (MLC2v) promoter, myosin heavy chain 7 promoter, myosin light chain 2 promoter, myosin light chain 4 promoter, a tropomyosin I promoter, α-actin promoter, α-myosin heavy chain (α-MHC) promoter, and cardiac Na+/Ca2+ exchanger (NCX1) promoter.

A polynucleotide of interest can also be combined with a condensing agent to form a gene delivery vehicle. The condensing agent may be a polycation, such as polylysine, polyarginine, polyornithine, protamine, spermine, spermidine, and putrescine. Many suitable methods for making such linkages are known in the art.

In an alternative embodiment, a polynucleotide of interest is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier which sequesters and protects its contents, for example, from degradative enzymes. Additionally, because a liposome is a synthetic structure, specially designed liposomes can be produced which incorporate desirable features. See Stryer, Biochemistry, pp. 236-240, 1975 (W.H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., Proc. Natl. Acad. Sci. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising growth factor polynucleotides such those described herein Liposomal preparations for use in the methods described herein include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. See also Felgner et al., Proc. Natl. Acad. Sci. USA 91: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

In some embodiments, provided herein are vector delivery systems that are capable of expressing the oligomeric, polymorphism or disease associated mutation-targeting sequences provided herein, such as vectors that express a polynucleotide sequence that express a polynucleotide sequence that is complementary to any or more of the target sequences provided in FIGS. 7-20. Included in particular embodiments are vectors that express siRNA or other duplex-forming RNA interference molecules.

In some embodiments, the vector is a nucleic acid construct (e.g., a polynucleotide molecule, such as a DNA molecule derived, for example, from a plasmid, bacteriophage, yeast or virus, into which a polynucleotide can be inserted or cloned). A vector may contain one or more unique restriction sites and can be capable of autonomous replication in a defined host cell including a target cell or tissue or a progenitor cell or tissue thereof, or be integrated with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

A vector or nucleic acid construct system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In some embodiments, the vector or nucleic acid construct is one which is operably functional in a mammalian cell, such as a cardiomyocyte. The vector can also include a selection marker such as an antibiotic or drug resistance gene, or a reporter gene (i.e., green fluorescent protein, luciferase), that can be used for selection or identification of suitable transformants or transfectants. Exemplary delivery systems may include viral vector systems (i.e., viral-mediated transduction) including, but not limited to, retroviral (e.g., lentiviral) vectors, adenoviral vectors, adeno-associated viral vectors, and herpes viral vectors, among others known in the art.

### Pharmaceutical Compositions

Pharmaceutical compositions described herein include the interfering RNA molecules, vectors and/or nucleic acids described herein and a pharmaceutically acceptable carrier or vehicle. The pharmaceutical compositions may further include additional agents for the treatment of HCM, DCM and/or LVNC.

A pharmaceutical composition described herein is formulated to be compatible with its intended route of administration. In certain embodiments, the pharmaceutical composition is administered via intra-venous injection. In some embodiments, the septal perforating artery can be selectively cannulated to target interventions to the interventricular septum.

Toxicity and therapeutic efficacy of the agents described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. In some embodiments, the dosage of such compounds lies within a range of circulating concentrations that include the ED50 with little or no toxicity. The dosage may vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Appropriate dosage agents depends upon a number of factors within the scope of knowledge of the ordinarily skilled physician, veterinarian, or researcher. The dose(s) of the small molecule will vary, for example, depending upon the identity, size, and condition of the subject or sample being treated, further depending upon the route by which the composition is to be administered.

### Kits

In some embodiments, provided herein is a kit comprising an interfering RNA molecule, a nucleic acid and/or a vector disclosed or contemplated herein. In some embodiments, the kit comprises at least 2, 3, 4, 5, 6, 7, 8, 9, 10, 11, 12, 13, 14, 15, 20, 25 or 30 different interfering RNA molecules disclosed or contemplated herein, wherein each different RNA molecule targets a different polymorphism or mutation on a gene selected from MYH7, MYBPC3, TNNT2, TNNI3, ACTC1, MYL2, MYL3 and TPM1. In some embodiments, the kit further includes instructions for use.

In some embodiments, the kit comprises at least 2, 3, 4, 5, 6, 7, 8, 9, 10, 11, 12, 13, 14, 15, 20, 25 or 30 nucleic acid molecules encoding different interfering RNA molecules disclosed or contemplated herein, wherein each different RNA molecule targets a different polymorphism or mutation on a gene selected from MYH7, MYBPC3, TNNT2, TNNI3, ACTC1, MYL2, MYL3 and TPM1. In some embodiments, the kit further includes instructions for use.

In some embodiments, the kit comprises at least 2, 3, 4, 5, 6, 7, 8, 9, 10, 11, 12, 13, 14, 15, 20, 25 or 30 vectors encoding different interfering RNA molecules disclosed or contemplated herein, wherein each different RNA molecule targets a different polymorphism or mutation on a gene selected from MYH7, MYBPC3, TNNT2, TNNI3, ACTC1, MYL2, MYL3 and TPM1. In some embodiments, the kit further includes instructions for use.

### Therapeutic Methods

Provided herein is a method of preventing or treating HCM, DCM or LVNC in a subject who has in their genome a first allele containing a disease-causing mutation, a (e.g., an HCM-causing mutation, a DCM-causing mutation or a LVNC-causing mutation listed in FIG. 8, 10, 12, 14, 16, 18 or 20). In certain embodiments, the subject further comprises a second allele that does not contain an a disease-causing mutation. In some embodiments, the method comprises administering to the subject an interfering RNA molecule described herein (e.g., a siRNA molecule or a shRNA molecule) that selectively inactivates the transcript encoded by the first allele compared to the transcript encoded by the second allele.

In some embodiments, the method comprises administering to the subject more than one different interfering RNA molecule described herein, wherein each interfering RNA molecule selectively inactivates the transcript encoded by the first allele compared to the transcript encoded by the second allele. In some embodiments, each interfering RNA molecule targets a different polymorphism or mutation present on the transcript encoded by the first allele but that is not present on the transcript encoded by the second allele. In some embodiments, the interfering RNA molecule targets a polymorphism or mutation present on the transcript encoded by the first allele but that is not present on the transcript encoded by the second allele (e.g., a polymorphism or mutation listed in FIG. 7, 9, 11, 13, 15, 17 or 19). In some embodiments, the interfering RNA molecule targets the disease-causing mutation. In some embodiments, the interfering RNA molecule targets a polymorphism present on the transcript encoded by the first allele that is not a disease-causing mutation.

In some embodiments, the inhibitory RNA molecule is delivered in a vector that has a tropism for cardiac tissue. In some embodiments, the vector is an adeno-associated virus (AAV). In some embodiments, expression of the inhibitory RNA molecule and/or the vector is driven by a cardiac-specific promoter. In certain embodiments, the cardiac-specific promoter is a cardiac specific troponin T promoter.

In some embodiments, the subject has had their MYH7, MYBPC3, TNNT2, TNNI3, ACTC1, MYL2, MYL3 and/or TPM1 genes sequenced before undergoing therapeutic treatment. Any sequencing method available in the art can be used. In some embodiments, the sequencing is performed using chain termination sequencing, sequencing by ligation, sequencing by synthesis, pyrosequencing, ion semiconductor sequencing, single-molecule real-time sequencing, dilute-‘n’-go sequencing and/or 454 sequencing. In some embodiments, the inhibitory RNA molecule administered is selected based on the sequencing results.

The pharmaceutical compositions described herein can be delivered by any suitable route of administration in particular embodiments. In certain embodiments, the pharmaceutical compositions are delivered generally (e.g., via oral or parenteral administration). In certain other embodiments the pharmaceutical compositions are delivered locally through direct injection into a tumor by direct injection into the heart or the heart's blood supply.

In some embodiments, the subject pharmaceutical compositions described herein will incorporate the substance or substances to be delivered in an amount sufficient to deliver to a patient a therapeutically effective amount of an incorporated therapeutic agent or other material as part of a prophylactic or therapeutic treatment. The desired concentration of the active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

All publications, including patents, applications, and GenBank Accession numbers mentioned herein are hereby incorporated by reference in their entirety as if each individual publication or patent was specifically and individually indicated to be incorporated by reference. In case of conflict, the present application, including any definitions herein, will control.

The invention now being generally described, it will be more readily understood by reference to the following examples, which are included merely for purposes of illustration of certain aspects and embodiments of the present invention, and are not intended to limit the invention.

### EXEMPLIFICATION

### Methods

### Mouse Protocols

All mice were maintained and studied using protocols approved by the animal Care and Use Committee of Harvard Medical School. Studies used male heterozygous MHC^{403/+} mice that were in 129SvEv background or heterozygous MHC^{403/+} F1 offspring male mice on the 129SvEv and FVB background. Viruses were injected via a single 50 μl bolus, using a 30G needle inserted through the diaphragm by a subxiphoid approach into the inferior mediastinum, avoiding the heart and the lung. Cyclosporine A (CsA, Sandimmune (100 mg cyclosporine capsule), Novartis, N.Y., USA)) was administered via oral chow that contains CsA (1 mg/g).

### Cell Culture and Transfection

293T cells were cultured at 37° C. with 5% CO₂ and maintained in DMEM, supplemented with 10% heat-inactivated FBS, 0.1 mM MEM nonessential amino acid, 5,000 units per ml penicillin-streptomycin. Transfection of RNAi constructs and overexpression plasmids was performed using Lipofectamine 2000 (Invitrogen) according to manufacturer's instructions.

### AAV-9 Production and Purification

AAV vectors were packaged into AAV-9 capsid by the triple transfection method using helper plasmids pAdAF6 and plasmid pAAV2/9 (Penn Vector Core). Fifty μg of plasmid DNA was used per 15 cm cell culture plate. Three days after transfection, AAV vectors were purified by Optiprep density gradient medium (D-1556, Sigma) by centrifugation and stored at −80° C.

### RNAi Vector Construction

Constructs for Myh6 R403Q were designed to target 21 base-pair gene-specific regions. Oligonucleotides were cloned into pCAG-miR30. The sequences targeted by RNAi are as follow: 403m RNAi, ccctcaggtgagggtggggac (SEQ ID NO: 12); 403i, cactcaggtgagggtggggac (SEQ ID NO: 13); 129i, ccactttggagctactggaaa (SEQ ID NO: 14). The sequence against LacZ, gactacacaaatcagcgattt (SEQ ID NO: 15) was used as control RNAi. The miR-30 cassette was inserted 3′ of the EGFP gene in AAV vector.

### RNA-Seq and Analysis

Hearts from mouse (strain 129SvEv) were rapidly isolated, placed in room temperature PBS to evacuate blood, and then immersed in RNALater (Qiagen) at room temperature. Two micrograms of total ventricular RNA was used to construct RNAseq sequencing libraries. In brief, polyA RNA is selected on oligo-dT magnetic beads, converted to cDNA with reverse transcriptase, made double stranded DNA, flush ended, and ligated to double strand Illumina sequencing adapters. Size selected 150-250 by fragments were isolated from acrylamide gels before amplification and sequenced (50 base, paired ends) using the Illumina HiSeq2000.

### Quantification of Myocardial Fibrosis

Hearts were excised from isoflurane-euthanized mice, washed in PBS, fixed overnight in 4% paraformaldehyde, and embedded in paraffin. After serial sectioning of hearts (apex to base), eight evenly distributed 5 μm sections were stained with Masson trichrome. Heart sections were scanned by BZ-9000 Generation II (Keyence). Fibrosis areas within sections were measured by software BZ-II Analyzer (Keyence). The percentage of total fibrosis area was calculated as the summed blue-stained areas divided by total ventricular area.

### Immuno-Staining and Analysis

Histochemical analyses were performed on heart sections fixed in 4% paraformaldehyde overnight. Sections were treated with xylene (to remove paraffin), re-hydrated, and permeabilized in 0.1% (v/v) Triton-X100 in PBS. Sections were incubated with primary antibodies applied at 1:200 dilution (unless otherwise indicated) in 0.1% (w/v) BSA in PBS overnight at 4° C. and non-specific antibody binding was blocked by 1.5% (v/v) donkey serum in PBS. Primary antibodies included: cardiac troponin-I (goat anti-Tnni3, Abcam ab56357, 1:200), GFP (chicken anti-GFP, Abcam ab13970, 1:200).

### Echocardiogram and Surface Electrocardiogram (ECG)

Echocardiogram data were obtained using Vevo 770 High-Resolution In Vivo Micro-Imaging System and RMV 707B scan-head (VisualSonics Inc.) as previously described. The images were acquired as 2D and M-mode (left parasternal long and short axes) and measurements were averaged from 3 consecutive heart beats of M-mode tracings as recommended by the American Society of Echocardiography's Guidelines. LV end-diastolic diameter (LVEDD), LV end-systolic diameter (LVESD), and wall thickness (LVWT) were attained by short axis image and left atrial diameter (LA) and aortic root diameter (Ao root) were measured by long axis image. LV fractional shortening (%) was calculated as follows: (LVEDD−LVESD)/LVEDD×100. Surface ECG were recorded with GE/Marquette CardioLab 7000 EP recording system.

### Statistical Analyses

Statistical analysis of data was performed by t-test, false discovery rate (FDR) and ANOVA for multiple comparisons.

### Example 1

### Generation of RNAi Capable of Allele-Specific RNA Silencing

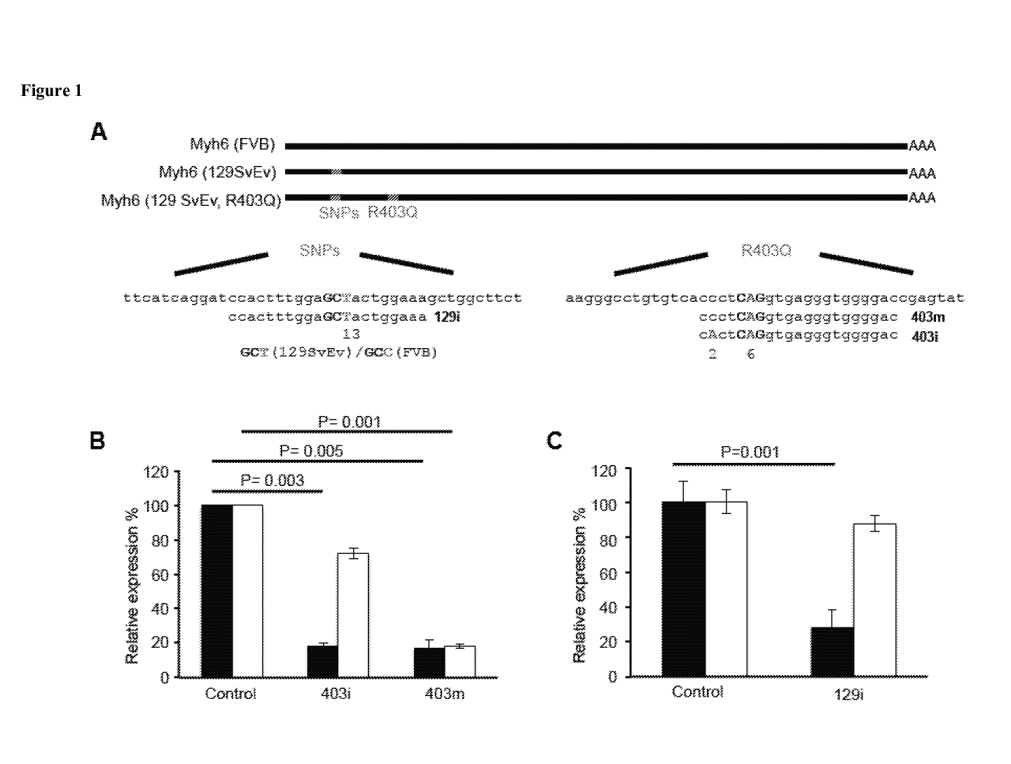

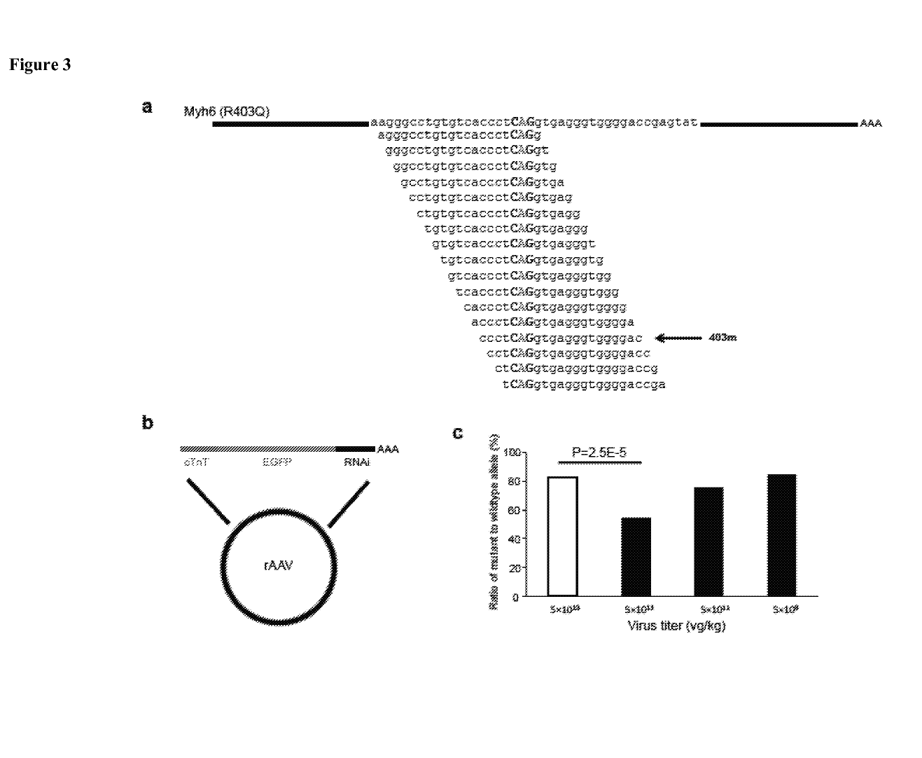

Seventeen unique RNAi constructs were produced. Each RNAi construct was co-transfected with a plasmid carrying the Myh6 R403Q mutant gene into 293T human embryonic kidney cells (FIG. 3a). One RNAi construct, designated 403m, significantly reduced Myh6 R403Q expression (FIG. 1A, B). To assess its specificity, wild-type or mutant Myh6 was transfected into 293T cells with 403m constructs. Because there was significant silencing (˜80%) of both wild-type and mutant Myh6 expression, an additional mismatch was introduced into the 403m RNAi construct (designated 403i; FIG. 1A). 403i had modest reduction (˜20%) of wild-type Myh6 expression, but retained approximately 80% reduction in the expression of Myh6 R403Q transcript in 293T cells (FIG. 1B).

### Example 2

### Generation of a Cardiac-Selective Vector

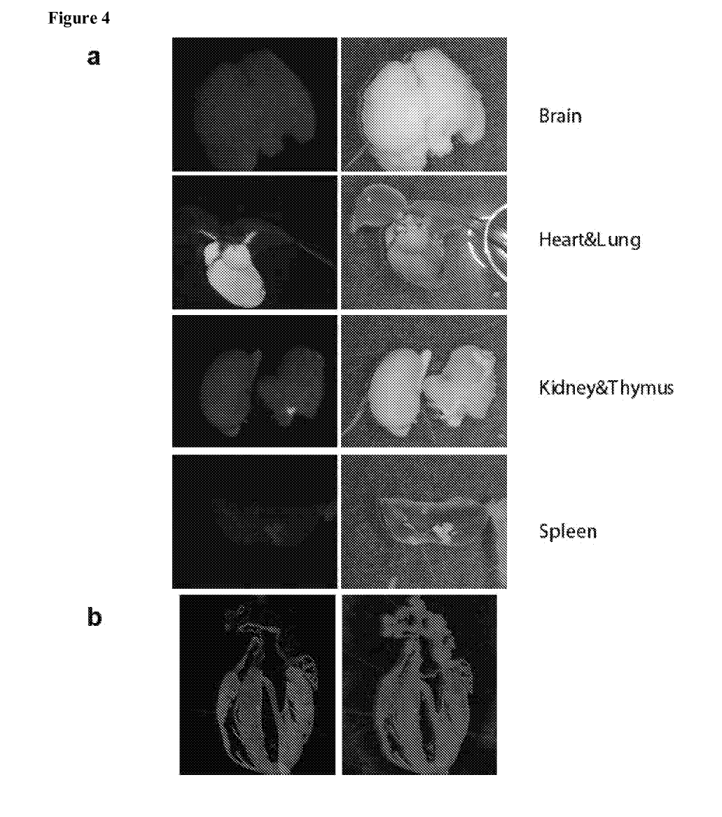

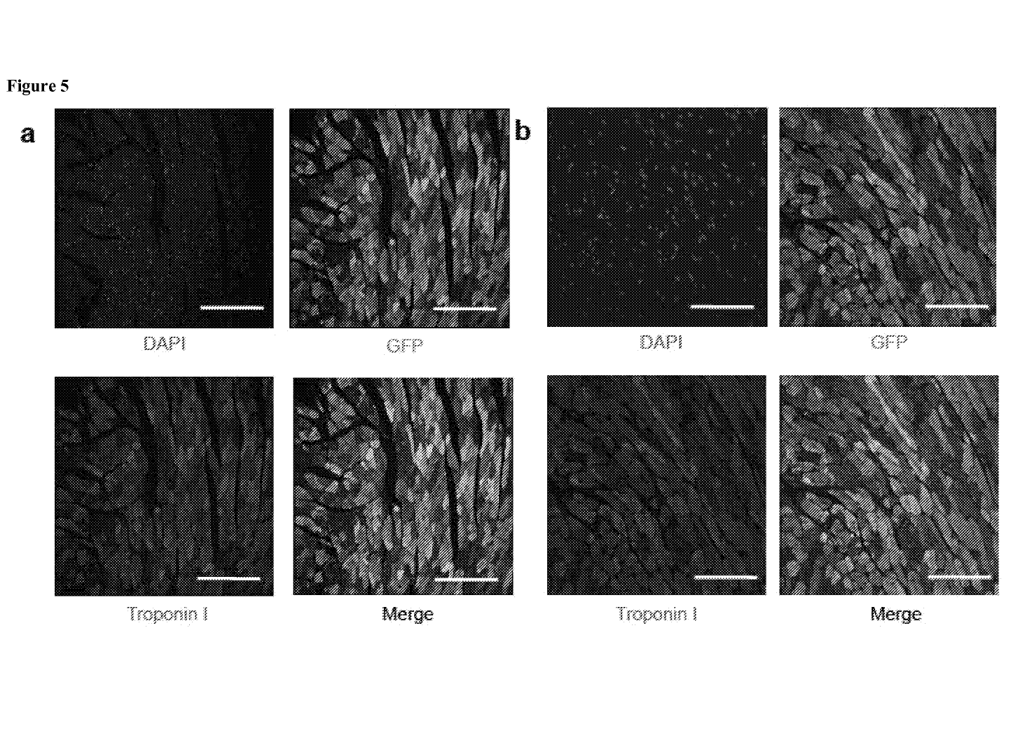

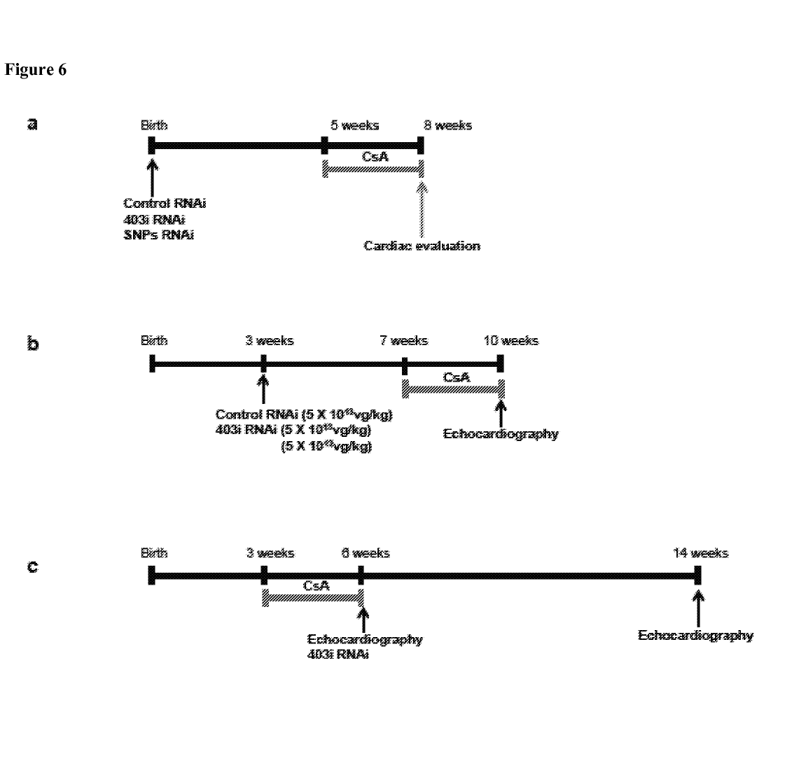

To ascertain the cardiac selectivity of AAV-9-cTnT vector, enhanced green fluorescent protein (EGFP) was used (FIG. 3b). Virus was injected (5×10¹³ vector genomes (vg)/kg) into the thoracic cavity of one-day old mice and after 3 weeks, all organs were dissected and EGFP expression was assessed by fluorescence microscopy. EGFP expression occurred exclusively in the heart and was absent in other organs including the brain, lung and spleen (FIG. 4). EGFP expression was present within 48 hours after virus transduction and remained robust for 12 months (FIG. 4, 5).

### Example 3

### In Vivo Allele Specific Gene Silencing

403i shRNA or control shRNA (denoted 403i RNAi and control RNAi, respectively) was engineered into the AAV-9-cTnT-EGFP-RNAi vector so that all cells expressing EGFP would also express shRNAs. To assess the efficacy of 403i shRNA in vivo, variable amounts of 403i RNAi-encoding viruses (5×10⁹, 5×10″ and 5×10¹³ vg/kg) were injected into the thoracic cavity of one-day old mice. Two weeks after viral transduction, total RNA extracted from each left ventricle (LV) was individually analyzed by RNA-seq. Sequencing reads that corresponded to Myh6 R403Q or wild-type Myh6 were counted and visualized using Integrative Genomics Viewer (IGV, Broad Institute, MA). The expression of Myh6 was comparable in LV tissues after transduction with control RNAi (12,118 reads per million transcripts) and 403i RNAi (11,675 reads per million transcripts), indicating that the wild-type allele was not silenced in vivo. In contrast, the ratio of Myh6 R403Q to Myh6 (wild-type) reads varied between 403i RNAi titers. Only the highest titer (5×10¹³ vg/kg) resulted in a significant reduction (28.5%) in the relative expression of Myh6 R403Q compared to wild-type Myh6 transcripts (P=2.5E-5) (FIG. 3c).

### Example 4

### Treatment of HCM by Allele Specific Gene Silencing

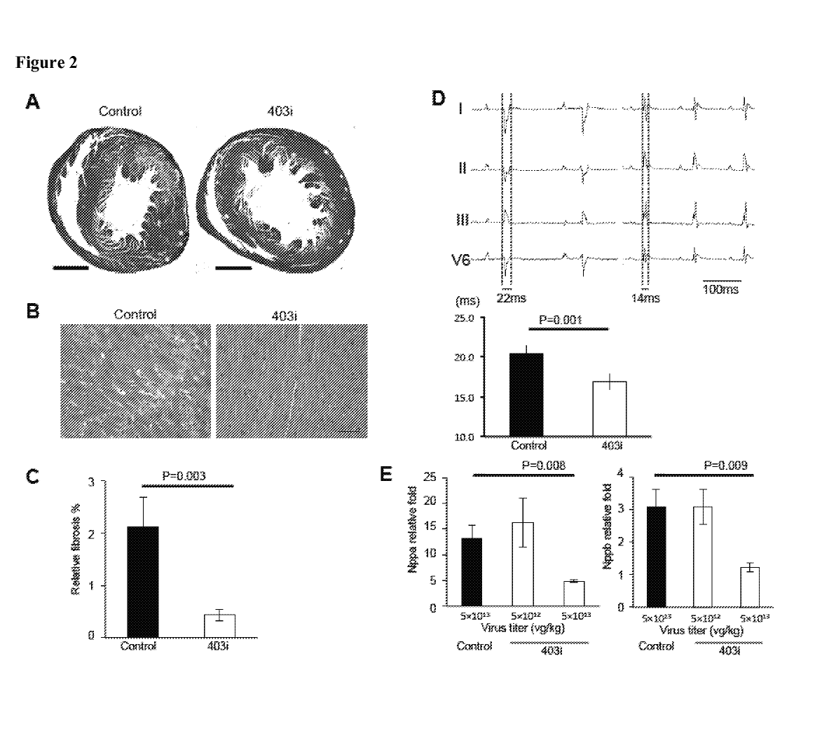

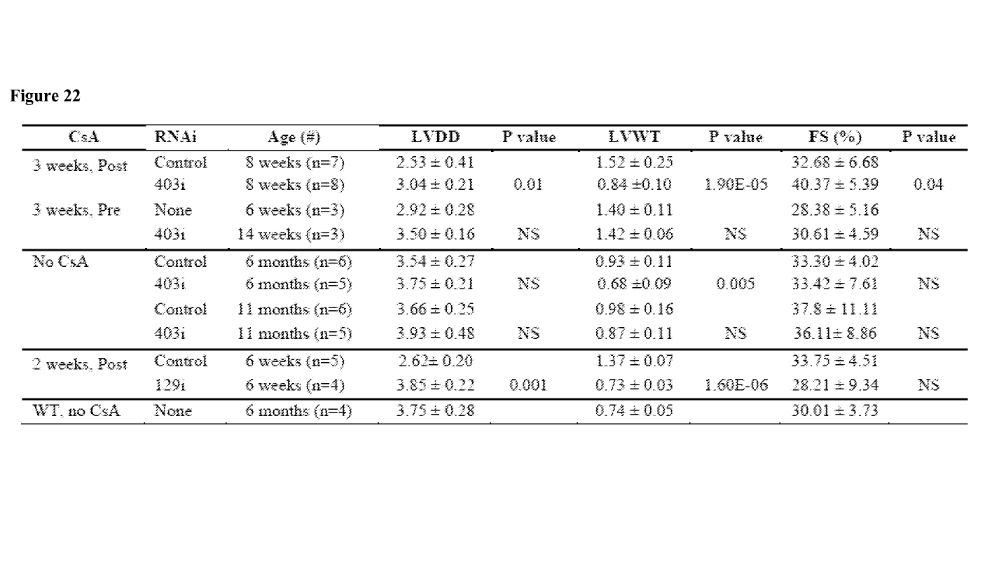

To assess the impact of silencing Myh6 R403Q on HCM development, virus encoding the 403i RNAi cassette (n=8) or control RNAi cassette (n=7) was injected into the thoracic cavity of one-day old male MHC^{403/+} mice. At 5 to 6 weeks of age, all mice were given cyclosporine A (CsA) for 3 weeks to accelerate the emergence of HCM histopathology. Mice were serially evaluated by echocardiography and at sacrifice, the hearts were analyzed by histopathology. After CsA treatment, control RNAi-transduced mice had LV hypertrophy and severe HCM histopathogy (FIG. 2A), similar to non-transduced, CsA-treated MHC^{403/+} mice. In contrast, CsA-treated MHC^{403/+} mice transduced with 403i RNAi did not develop HCM (FIG. 22, upper panel). The left ventricular wall thickness (LVWT) of 403i RNAi-transduced mice (0.84±0.10 mm) was significantly less than that of mice transduced with control RNAi (1.52±0.25 mm, P=1.9E-5) and comparable to the LVWT of wild-type mice (0.74±0.05 mm, NS) (12). Myocardial disarray (FIG. 2B) was absent and fibrosis (FIG. 2C) was significantly reduced in 403i RNAi-transduced mice (0.43±0.11%) compared to control RNAi-transduced hypertrophic ^{403/+} mice (2.12±0.57%, P=0.003). QRS interval prolongation, an electrocardiographic feature of LV hypertrophy, was present in mice transduced with control RNAi (20.5±1.2 ms) but not in mice transduced with 403i RNAi (16.9±1.4 ms, P=0.001) (FIG. 2D). Additionally, the expression of prototypic LV hypertrophy markers Nppa and Nppb were 2.5 fold higher in mice transduced with control RNAi compared to 403i RNAi (FIG. 2E).

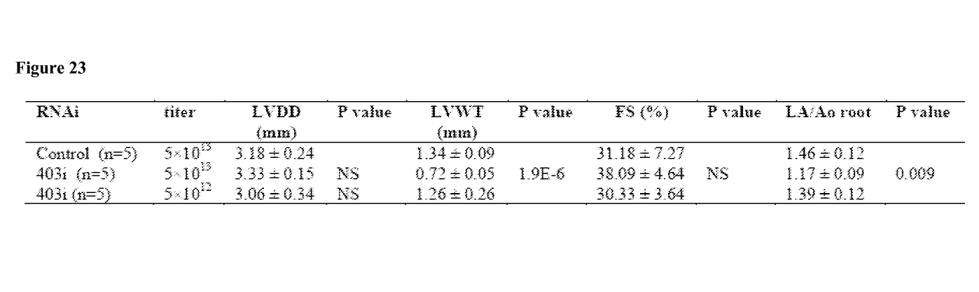

To assess if the early age at transduction and/or viral titer influenced HCM development, high (5×10¹³ vg/kg) and low (5×10¹² vg/kg) titer viruses of 403i RNAi were injected into 3-week old MHC^{403/+} mice (n=5). At 4 weeks, mice were treated with CsA for 3 additional weeks followed by echocardiography to assess LVWT and diastolic (relaxation) performance (left atrial diameter normalized to the aortic root diameter) which becomes abnormal early in HCM. Mice transduced with high viral titers of control RNAi or low viral titers of 403i RNAi had both LV hypertrophy and diastolic dysfunction (FIG. 23). In contrast, mice transduced with high titer 403i RNAi virus had neither hypertrophy (LVWT=0.72±0.05 mm, P=1.9E-6 compared to control RNAi) nor diastolic dysfunction (1.17±0.09, P=0.009 compared to control RNAi).

Using the high titer virus, whether 403i RNAi-transduction could alter established HCM was examined by pretreating MHC^{403/+} mice with CsA for 3 weeks to induce hypertrophy (LVWT, 1.40±0.11 mm) prior to viral transduction. Echocardiography assessments at two months after 403i RNAi (n=3) transduction showed no change in LVWT (FIG. 22, middle panel).

To determine whether 403i RNAi-transduction affected the pathologic LV remodeling that slowly emerges in MHC^{403/+} mice with age in the absence of CsA, LV hypertrophy was monitored in mice transduced with a single high titer dose of 403i RNAi (n=5) or control RNAi (n=6) on day one of life. At 6 months, mice transduced with control RNAi had LV hypertrophy (LVWT, 0.93±0.11 mm). There was no LV hypertrophy in mice transduced with 403i RNAi (LVWT=0.68±0.09 mm, P=0.004) and LVWT was indistinguishable from wild-type mice (LVWT, 0.74±0.05 mm, P=NS). LV hypertrophy emerged in 403i RNAi-transduced mice by 11 months of age (LVWT, 0.87±0.11 mm) and was comparable to that observed in control RNAi-transduced mice.

Whether a single RNAi might silence different, patient-specific mutations in the same gene, by targeting a nearby single nucleotide polymorphism (SNP) that distinguished the mutant from wild-type alleles was examined. To test this model, male F1 offspring were produced from 129SvEv MHC^{403/+} and wild-type FVB crosses. An RNAi that targeted a 129SvEv SNP on the Mhy6 allele (designated 129i, FIG. 1A) was constructed and transfected with Mhy6 R403Q (129SvEv) or wild-type Myh6 (FVB) plasmids into 293T cells. The 129i RNAi decreased Mhy6 R403Q levels by 75% and reduced wild-type Myh6 (FVB) by only 15% (FIG. 1C). AAV-9-cTnT-EGFP-129i virus was produced and transduced (5×10¹³ vg/kg) into one-day old male F1 MHC^{403/+} mice with 129i RNAi (n=4) or control RNAi (n=5). At 4 weeks of age, mice were treated with CsA for 2 weeks and studied by echocardiography. Control RNAi-transduced mice developed LV hypertrophy (LVWT=1.37±0.03 mm) but not MHC^{403/+} mice transduced with 129i RNAi (LVWT=0.73±0.07 mm; P=1.6E-6, FIG. 22). These studies demonstrated that one RNAi construct which targeted a SNP that demarcates mutant and wild-type alleles could be used to silence distinct HCM mutations in a gene or to augment mutation-specific RNAi.

We claim:

1. A method of preventing or treating hypertrophic cardiomyopathy (HCM) in a subject having in their genome a first MYH7 allele comprising an HCM-causing mutation and a second MYH7 allele that does not comprise the HCM-causing mutation, the method comprising administering to the subject an interfering RNA molecule that selectively inactivates the transcript encoded by the first MYH7 allele compared to the transcript encoded by the second MYH7 allele.

2. The method of claim 1, wherein the HCM-causing mutation is an HCM-causing mutation listed in FIG. 8.

3. The method of claim 1, wherein the HCM-causing mutation is an R403Q mutation.

4. The method of claim 1, wherein the interfering RNA molecule targets a polymorphism or mutation present on the transcript encoded by the first MYH7 allele but that is not present on the transcript encoded by the second MYH7 allele.

5. The method of claim 4, wherein the polymorphism or mutation is a polymorphism or mutation listed in FIG. 7.

6. The method of claim 4, wherein the interfering RNA molecule targets the HCM-causing mutation.

7. The method of claim 4, wherein the interfering RNA molecule targets a polymorphism present on the transcript encoded by the first MYH7 allele that is not the HCM-causing mutation.

8. The method of claim 7, wherein the HCM-causing mutation is an HCM-causing mutation listed in FIG. 8.

9. The method of claim 1, wherein the interfering RNA molecule comprises a nucleic acid sequence of 21 nucleotides in length, wherein 20 nucleotides of the nucleic acid sequence are complementary to the transcript of the first MYH7 allele and no more than 19 nucleotides of the nucleic acid sequence are complementary to the transcript of the second MYH7 allele.

10. The method of claim 1, wherein the interfering RNA molecule inactivates the transcript of the first MYH7 allele at least 2 times as much as it inactivates the second MYH7 allele.

11. The method of claim 1, wherein the method further comprises the step of sequencing the first MYH7 allele and the second MYH7 allele before administering to the subject the interfering RNA molecule.

12. The method of claim 4, wherein the interfering RNA molecule comprises a nucleotide sequence that is complementary to a nucleotide sequence of the first allele that includes the polymorphism or mutation except for a single nucleotide mismatch at a position outside of the polymorphism or mutation.

13. The method of claim 1, wherein the interfering RNA molecule is an siRNA molecule or an shRNA molecule.

14. The method of claim 1, wherein the inhibitory RNA molecule is delivered in a vector that has a tropism for cardiac tissue.

15. The method of claim 14, wherein the vector is an adeno-associated virus (AAV).

16. The method of claim 1, wherein expression of the inhibitory RNA molecule is driven by a cardiac-specific promoter.

17. The method of claim 16, wherein the cardiac-specific promoter is a cardiac specific troponin T promoter.

18. The method of claim 1, wherein the method comprises administering to the subject more than one different interfering RNA molecule, wherein each interfering RNA molecule selectively inactivates the transcript encoded by the first MYH7 allele compared to the transcript encoded by the second MYH7 allele.

19. The method of claim 18, wherein each interfering RNA molecule targets a different polymorphism or mutation present on the transcript encoded by the first MYH7 allele but that is not present on the transcript encoded by the second MYH7 allele.

20. The method of claim 19, wherein the targeted polymorphisms or mutations are polymorphisms or mutations listed in FIG. 7.

21-80. (canceled)

81. A method of preventing or treating hypertrophic cardiomyopathy (HCM), dilated cardiomyopathy (DCM) or Left Ventricular Non-Compaction (LVNC) in a subject having in their genome a first MYL3, MYH7, TNNI3, TNNT2, TPM1 or ACTC1 allele comprising an HCM-causing, a DCM-causing or a LVNC-causing mutation and a second MYL3, MYH7, TNNI3, TNNT2, TPM1 or ACTC1 allele that does not comprise the HCM-causing, the DCM-causing or the LVNC-causing mutation, the method comprising administering to the subject an interfering RNA molecule that selectively inactivates the transcript encoded by the first MYL3, MYH7, TNNI3, TNNT2, TPM1 or ACTC1 allele compared to the transcript encoded by the second MYL3, MYH7, TNNI3, TNNT2, TPM1 or ACTC1 allele.

82-260. (canceled)

261. An interfering RNA molecule or antisense oligonucleotide that selectively inhibits expression of a MYH7, MYL3, TNNI3, TNNT2, TPM1 or ACTC1 transcript comprising a MYH7, MYL3, TNNI3, TNNT2, TPM1 or ACTC1 polymorphism or mutation compared to a MYH7, MYL3, TNNI3, TNNT2, TPM1 or ACTC1 transcript not comprising the MYH7, MYL3, TNNI3, TNNT2, TPM1 or ACTC1 polymorphism or mutation.

262-341. (canceled)
